(19) United States
(12) Patent Application Publication
(10) Pub. No.: US 2006/0003312 A1
(43) Pub. Date: Jan. 5, 2006

(54) Circulating stem cells and uses related thereto

(75) Inventors: Helen M. Blau, Stanford, CA (US); Timothy Brazelton, Cupertino, CA (US); Mark A. LaBarge, Davis, CA (US); James M. Weimann, Palo Alto, CA (US)

Correspondence Address: FISH & NEAVE IP GROUP;ROPES & GRAY LLP, ONE INTERNATIONAL PLACE, BOSTON, MA, 02110-2624, US

(73) Assignee: Stanford University, Palo Alto, CA (US)

(21) Appl. No.: 11/120,581

(22) Filed: May 2, 2005

Related U.S. Application Data
(63) Continuation of application No. PCT/US03/35284, filed on Nov. 3, 2003.
(60) Provisional application No. 60/422,959, filed on Nov. 1, 2002.
(60) Provisional application No. 60/426,976, filed on Nov. 15, 2002.

(51) Int. Cl. C12Q 1/68; C12Q 1/00

(52) U.S. Cl. 435/4; 435/6

(57) ABSTRACT

The disclosure provides, inter alia, methods for enhancing the contribution of circulating stem cells to a target tissue. Such methods may be useful for treating a variety of disorders. In a preferred embodiments, circulating stem cell contribution is enhanced by causing damage to the target tissue, or by administering an agent that mimics an aspect of a damage response. The disclosure also provides methods for monitoring the contribution of circulating stem cells to a target tissue, and for developing agents that modulate such contribution.

### RELATED APPLICATIONS

This application is a continuation of International Patent Application PCT/US03/35284, filed Nov. 3, 2003, designating the U.S., which claims the benefit of the filing date of U.S. Provisional Application 60/422,959, filed Nov. 1, 2002 and entitled “Circulating Stem Cells and Uses Related Thereto” and U.S. Provisional Application 60/426,976, filed Nov. 15, 2002 and entitled “Circulating Stem Cells and Uses Related Thereto”, both of which are incorporated by reference herein in entirety. International Application PCT/US03/35284 was published under PCT Article 21(2) in English.

### FUNDING

Work described herein was funded, in part, by grant nos. AG20961 and AG09521 from the National Institutes of Health. The United States government has certain rights in the invention.

### BACKGROUND OF THE INVENTION

The replacement of damaged organs and tissues is a major problem in health care. Most organs and tissues regenerate poorly in mammals, and therapeutic agents to elicit repair in damaged or diseased tissues are generally unavailable or ineffective. Human muscle tissue, for example, has a limited capacity for regeneration. Cardiac muscle tissue following injury, such as a myocardial infarction, generally fails to regenerate. Consequently, some types of muscle injuries or muscle disorders require a lengthy healing time, and in other instances muscle damage is not reparable. Other tissues such as neural tissue, cartilaginous tissues and many solid organs are also poorly regenerative.

Artificial materials, such as replacement joints, and mechanical devices, such as renal dialysis machines, have provided a partial cure for some types of tissue damage. However, each of these artificial materials is associated with undesirable side-effects or deficiencies. For example, dialysis causes a considerable decrease in quality of life for many patients. Artificial joints tend to wear out and may be difficult or impossible to replace.

Transplants with organs or tissues from other individuals are effective in some instances. For example, kidney, heart, liver, lung and bone marrow have been successfully transplanted. However, the majority of transplant recipients of life sustaining organs die from factors associated with the transplant, typically, either from direct graft failure (e.g., acute rejection, chronic rejection, etc.) or from factors related to the immunosuppressive regimen (e.g., infection, direct organ toxicity, etc.). Furthermore, transplant therapies are often available to only a select group of patients, because the supply of suitable organs and tissues is sharply limited and unpredictable, being largely dependent on post mortem donation from accident victims. Additionally, transplant therapy is very costly, and transplant recipients must receive immunosuppressive drug therapy in order to avoid rejection due to the genetic differences between donor and recipient. Efforts have been made to solve the supply problem through the use of organs obtained from non-human species, such as pigs, but immuno-incompatibility remains a major problem. Xenotransplantation also poses the danger of introducing new viruses that are pathogenic to humans and might emerge from long term association with an organ from a different species. For example, recent findings show that porcine endogenous retroviruses can infect human cells in vitro.

In the case of skeletal muscle tissue, transplantation would be particularly desirable for patients undergoing plastic or reconstructive surgery, as well as patients suffering from muscle dystrophy. Transplantation of autologous or allogenic tissue has been used, but only with limited success. Donor tissue is extremely scarce, and treatment of transplant recipients with immunosuppressant drugs creates substantial health risks for the transplant recipient.

An alternative strategy is the use of stem cells to promote tissue growth in vivo or to generate cultured tissues for transplantation (reviewed by Vogel, Science 283:1432-1434 (1999)). Stem cells are cells that are capable of self-renewal and give rise to cells of more specialized function (reviewed by Blau, Cell 105:829-841 (2001); Fuchs and Segre, Cell 100:143-155 (2000); and by Weissman, Cell 100:157-168 (2000)). For example, mammalian bone marrow contains a range of hematopoietic (blood-forming) stem cells. This feature has been exploited clinically in bone marrow transplantation, by allowing these stem cells to repopulate the bone marrow after removal of the diseased cells.

The two main categories of stem cells are those derived from embryos, the embryonic stem cells (ESC), and those present in adults. ESCs have substantial plasticity and are able to give rise to a wide range of cells. However, when injected in a pluripotent state into animals ESCs generate tumors, most notably teratomas. Thus, ESCs must be differentiated into mature cells ex vivo, an inefficient process since even the best differentiation procedures result in heterogeneous populations of cells with distinct fates. Most importantly, any population of cells derived from ESC must be carefully screened to ensure that no non-differentiated, pluripotent, tumorogenic ESC remain, a substantial challenge to the development of clinical therapies utilizing ESC. In addition, ethical concerns, immune reactions, and the standard quality control issues surrounding the delivery of ex vivo cultured cells pose additional challenges to the use of ESC.

Various types of stem cells have been detected in adults including tissue specific stem cells and pluripotent bone marrow derived cells including the marrow stromal cells, mesenchymal stem cells, and other populations of bone marrow derived cells of unknown identity. Tissue specific stem cells reside in various tissues where they are able to proliferate and generate specific cell types to maintain or repair that tissue. For example, the tissue specific stem cells in skeletal muscle, called satellite cells, proliferate in response to skeletal muscle damage and incorporate into damaged myofibers or, through a coordinated process with other muscle precursor cells, form myofibers de novo. One major advantage of adult stem cells is that, since they exist in adult humans, the right combination of stimuli may be able to be recruit them to perform regenerative functions at a level greater than that seen in typical physiological and disease processes.

Efforts are underway to use stem cells to treat diseases ranging from diabetes to Parkinson's disease. Yet, despite the enormous potential of stem cells, therapeutic interventions based on stem cells have been difficult to develop. Controlling the developmental fate of stem cells in culture has been a significant challenge for stem cell researchers, as has the task of identifying stem cells that are suitable to give rise to tissues and cell types of interest.

An ideal solution would be to take advantage of, and possibly enhance, one or more of the endogenous mechanisms by which stem cells in an organism participate in regenerative functions. Several routes for achieving this objective are described below.

### SUMMARY

In certain aspects, the present invention relates to the discovery that endogenous or exogenous bone marrow derived stem cells (BMDSCs) contribute significantly in vivo to various tissues that are not of the traditional hematopoietic lineages. One pioneering finding disclosed herein is the discovery that bone marrow derived stem cells infiltrate skeletal muscle tissue, become muscle-specific stem cells (satellite cells) and give rise to mature, differentiated skeletal myocytes, and furthermore, that this process occurs in vivo at rates far higher than previously demonstrated or expected. Another pioneering finding disclosed herein is the discovery that bone marrow derived stem cells infiltrate neural tissue and fuse with mature neurons to form heterokaryons; again, this process occurs more frequently in vivo than expected. A further pioneering finding presented herein is the discovery that damaged tissues show increased recruitment of bone marrow derived stem cells, thus demonstrating for the first time that the recruitment of pro-regenerative stem cells to tissues can be regulated in vivo and that endogenous, inducible factors regulate the process of stem cell recruitment and tissue regeneration.

Accordingly, an aspect of the invention provides methods for causing circulating stem cells (“CSCs”), and particularly BMDSCs, to enter a target tissue and become tissue-localized stem cells and/or fuse with cells of the target tissue to generate heterokaryons. In certain embodiments, the tissue-localized stem cells derived from the CSCs proliferate and give rise to mature cells of the target tissue. In certain embodiments, heterokaryons formed by cell fusion are endowed with advantageous properties derived from the fused stem cell. In certain embodiments, damage, or damage-like signals, may be used to enhance the contribution of CSCs to a target tissue. In further aspects, the invention provides for the identification of agents that inhibit or promote contribution of CSCs to target tissues. An inhibitor may, for example, have therapeutic value in disorders characterized by non-cancerous over-proliferation or hypertrophy. In addition, the identification of an inhibitor may suggest pathways that can be modulated for the purposes of inhibiting or enhancing CSC contribution to target tissues. An enhancer may, for example, have therapeutic value in disorders characterized by cellular insufficiency of the target tissue, and the identification of an inhibitor may suggest pathways that can be modulated for the purposes of inhibiting or enhancing CSC contribution to target tissues.

Certain methods of the invention may be used for treatment or prophylaxis of disorders characterized by an insufficiency of mature cells (including functional mature cells) of a tissue. In addition certain methods of the invention may be used to generate or augment tissue, regardless of whether the tissue has been damaged or is otherwise deficient in mature cell function. In certain embodiments, the target tissue has experienced damage to the local tissue environment that inhibits regeneration of the affected tissue, such as fibrosis or the formation of a necrotic mass. In certain embodiments, the affected tissue is selected from the group consisting of: a neural tissue, a cartilaginous tissue, a cardiac tissue, a skeletal muscle tissue, a liver tissue, and a pancreatic tissue.

In certain embodiments, methods of the invention may be used to introduce genetically modified CSCs into a target tissue, and optionally the genetically modified CSCs produce a therapeutic polypeptide or therapeutic moiety.

By demonstrating that exogenous or endogenous stem cells can migrate from one location (e.g., the bone marrow) and become established at another location, the present application illuminates a series of mechanistic steps that may be manipulated so as to increase or decrease the contribution of stem cells to a target tissue. In addition, by demonstrating that damage functions to enhance CSC contribution to target tissues, the present application illuminates damage-related or damage-mimicking mechanisms that may be used for this purpose. Additionally, the present application demonstrates that damage (mild or severe, depending on the clinical desirability) can be used to enhance regeneration in a target tissue.

In certain aspects, methods of the invention comprise causing an increase in the number and/or quality of circulating stem cells in a subject. In certain embodiments, an increase in CSCs in the blood may be achieved by administering exogenous CSCs to the subject, particular BMDSCs. Optionally the exogenous CSCs are genetically modified. Optionally the exogenous CSCs are bone marrow-derived cells. In certain embodiments, an increase in CSCs in the blood may be achieved by causing an increased release of endogenous CSCs into the blood. Optionally, a method for the release of endogenous CSCs into the blood comprises administering to the subject an agent that stimulates production of bone marrow derived stem cells. Optionally, a method for the release of endogenous CSCs into the blood comprises administering to the subject an agent that stimulates movement of bone marrow derived stem cells into the bloodstream.

In certain aspects, methods of the invention comprise causing increased recruitment of CSCs to a target tissue. Optionally, increased recruitment may be achieved by treating the target tissue to create a niche for a tissue-localized stem cell. In certain embodiments a niche for tissue-localized stem cells may be created in a target tissue by eliminating one or more pre-existing tissue-localized stem cells, such as by damaging the target tissue (e.g., through irradiation, administration of a cell-targeted toxin, sufficient exercise, targeted ablation or microscopic or macroscopic mechanical disruption). In certain embodiments, a niche for a tissue-localized stem cell may be created by introducing into the target tissue a substance to which a tissue-localized stem cell adheres, such as a cell adhesion molecule or a basal membrane matrix of the target tissue. In certain embodiments, a niche is created by mechanical space creation in the target tissue. In certain embodiments, methods of the invention comprise administering to the subject an agent that facilitates movement of cells from the bloodstream into the target tissue. In additional embodiments, methods of the invention comprise administering a homing factor that facilitates recruitment of CSCs to the target tissue. A homing factor may, for example, be administered locally to the target tissue or designed so as to localize at the target tissue.

In certain aspects, methods of the invention comprise administering to the subject an agent that maintains the viability and/or developmental plasticity of a CSC. Optionally, an agent may stimulate or increase the developmental plasticity of a CSC. In certain embodiments, the agent increases the percentage of CSCs that are able to assume a developmental fate that is compatible with the target tissue.

In certain embodiments, methods of the invention comprise stimulating the proliferation and/or maturation of tissue-localized stem cells in the target tissue, particularly after CSCs have become incorporated into the target tissue as tissue-localized stem cells. Proliferation and/or maturation may be stimulated by, for example, exposing the tissue to a condition that damages mature cells of the tissue. In certain embodiments, methods of the invention comprise administering an agent that promotes maintenance of tissue-localized stem cells in the target tissue, often as quiescent cells.

In certain aspects the invention provides methods for causing circulating stem cells to enter a target tissue and become tissue-localized stem cells, wherein the methods employ two or more approaches disclosed herein. In certain embodiments, methods of the invention comprise increasing the circulating stem cells in the blood of the subject, and increasing recruitment of CSCs to a target tissue. In certain embodiments, methods of the invention may comprise increasing the circulating stem cells in the blood of the subject and treating the target tissue to create a niche for a tissue-localized stem cell. In certain embodiments, methods of the invention comprise increasing the circulating stem cells in the blood of the subject, and administering to the subject an agent that facilitates movement of cells from the bloodstream into the target tissue. In certain embodiments, methods of the invention comprise treating the target tissue to create a niche for a tissue-localized stem cell, and administering to the subject an agent that facilitates movement of cells from the bloodstream into the target tissue.

In certain aspects, the invention provides a method for generating cells of a non-hematopoietic target tissue in vivo from circulating stem cells, the method comprising causing circulating stem cells to become tissue-localized stem cells of the target tissue.

In certain aspects, the invention provides a method for generating cells of a non-hematopoietic target tissue in vivo from circulating stem cells, the method comprising causing circulating stem cells to fuse with cells of the target tissue, thereby forming heterokaryons.

In certain aspects the invention provides a method for treating a disorder characterized by an insufficiency of mature cells of a target tissue in a subject, the method comprising: administering to the subject an agent that enhances the contribution of bone marrow derived stem cells to the mature cells of the target tissue, wherein the target tissue is not of a hematopoietic lineage.

In certain aspects the invention provides a method for treating a disorder characterized by an insufficiency of mature cells of a target tissue in a subject, the method comprising: enhancing the contribution of bone marrow derived stem cells to the mature cells of the target tissue by creating a niche for formation of tissue-localized stem cells from a bone marrow derived stem cell, wherein the target tissue is not of a hematopoietic lineage.

In certain aspects, the invention provides a method for increasing the contribution of a bone marrow derived stem cell to a non-hematopoietic target tissue, the method comprising causing damage to and/or mimicking an effect of damage on the target tissue.

A target tissue may be essentially any tissue, although in certain embodiments the tissue is not a tissue of hematopoietic lineage. In certain embodiments the target tissue is a tissue with a well-defined tissue-localized stem cell. In certain embodiments, the target tissue is a solid organ, such as a liver, skeletal muscle, pancreas or heart. In certain embodiments, the target tissue is a tissue selected from the group consisting of: smooth muscle tissue, cartilaginous tissue, cardiac muscle tissue, liver tissue and pancreatic tissue. In certain embodiments the target tissue is neural.

In certain aspects the invention provides methods for assessing the contribution of CSCs to one or more target tissues. Certain methods described herein may be used for assessing the effects of test compounds on the contribution of CSCs to one or more tissues. In certain embodiments, methods described herein may be used to identify and enrich for CSCs that contribute to one or more target tissues. In certain embodiments, a method of the invention comprises: (a) causing endogenous or exogenous circulating stem cells to have a tracking marker; and (b) detecting the presence of the marked circulating stem cells or progeny or fusion cells derived therefrom in one or more regenerative target tissues. A tracking marker is generally any cell feature that may be used to distinguish the marked CSCs (and progeny and fusions thereof) from cells of the target tissue. Optionally, the tracking marker is a conditionally or constitutively expressed marker protein or a chromosomal feature that is distinct from the endogenous cells of the subject.

In certain embodiments, an assay of the invention for assessing the effects of test compounds on contribution of CSCs to a target tissue comprises: (a) causing endogenous or exogenous circulating stem cells to have a tracking marker; (b) administering the test agent to the subject; (c) detecting the presence of the marked circulating stem cells or progeny or fusion cells derived therefrom in one or more regenerative target tissues. The test agent may be administered before, after or simultaneous with part (a). In certain embodiments, the presence, absence or amount of CSCs in the one or more target tissues after treatment with the test agent may be compared to a suitable reference, such as a control subject that does not receive the test agent. In certain embodiments, methods for use in identifying a circulating stem cell that contributes to one or more target tissues in a subject comprises (a) administering a test cell population to the subject and causing the test cell population to have a tracking marker; and (b) detecting the presence of the exogenous circulating stem cells or progeny or fusion cells derived therefrom in one or more target tissues.

In certain embodiments, the invention provides methods for assessing the ability of a test treatment to alter the contribution of a stem cell to a target tissue in a subject, the method comprising: a) administering the test treatment to the subject; b) detecting the contribution of a stem cell to the target tissue; wherein the stem cell is of a distinct developmental lineage from the target tissue. A test treatment may be essentially any desired treatment of the subject, whether intended to increase or decrease stem cell contribution to the target tissue. A test treatment may, for example, comprise administering one or more test agents and/or exposing the subject to one or more conditions (e.g., creating an injury or model disease state in the subject. A preferred subject is a mouse or rat. Contribution of stem cells to the target tissue after treatment may be compared to a reference, which will generally be a measure of contribution in the absence of treatment. A preferred reference is a simultaneous control, optionally a similar, untreated tissue in the same subject.

In certain embodiments, the invention provides a method for assessing the ability of a test criterion to alter the contribution of a stem cell to a target tissue in a subject, the method comprising: a) detecting the contribution of a stem cell to the target tissue in a subject that has the test criterion; and b) comparing the detected contribution to a reference; wherein the stem cell is of a distinct developmental lineage from the target tissue. A test criterion is generally any feature of a subject (as compared to a control) that is of interest and/or is expected to affect contribution of stem cells to a target tissue.

Essentially any test agent may be tested for effects on the contribution of a CSC to a target tissue, such as a non-hematopoietic tissue, including but not limited to: small molecules; secreted, diffusible signaling molecules (e.g., peptide hormones, growth factors, cytokines, chemokines); extracellular, target tissue localized molecules; cell surface associated molecules (e.g., receptors, cell adhesion molecules); soluble extracellular portions of cell surface associated molecules; antibodies (particularly antibodies targeted to any of the preceding); antisense or siRNA nucleic acids (particularly those targeted to nucleic acids encoding any of the preceding proteins). Optionally, a test agent is derived from a damaged tissue or is a fractionated portion of an extract from a damaged tissue. Optionally, a test agent is an agent know to have one or more of the following properties: ability to mobilize BMDSCs or promote engraftment of exogenous BMDSCs; ability to promote CSC survival; ability to increase or maintain the developmental potential of a CSC; ability to promote extravasation of a circulating cell, such as a lymphocyte.

### BRIEF DESCRIPTION OF THE DRAWINGS

FIG. 1. Radiation and Exercise-Induced Damage Enhances the Contribution of GFP(+) Bone Marrow-Derived Cells to the Satellite Cell Niche
(A) Satellite cells from single muscle fibers isolated three weeks after irradiation of TA muscles and from contralateral non-irradiated controls were analyzed to determine the effect of irradiation on the endogenous satellite cells in the tissue-localized stem cell niche. 9.6 Gy elicits a 3-fold decrease and 18 Gy a 5-fold decrease in satellite cell number relative to non-irradiated controls (p<0.001). Three animals were analyzed for each irradiation condition. Differences in average fiber lengths among groups were not significantly different and did not contribute to differences in satellite cell number (1600±60 μm, p>0.5).
(B) GFP-expressing satellite cells derived from GFP(+) bone marrow were quantified on single isolated myofibers two months post-transplant. Transplant recipients that received no irradiation prior to transplant were compared with those that received 9.6 Gy. Lethal irradiation, 9.6 Gy, enhances GFP(+) satellite cell contribution (p<0.01).
(C) Fixed tissue transverse-sections of the transplant recipient and control TA muscles were analyzed for GFP(+) fibers, an indication of regeneration by GFP(+) bone marrow-derived cells. After 9.6 Gy one GFP(+) muscle fiber was detected among a total of 1589 fibers scored, none were detected in the control.
(D) GFP(−) endogenous satellite cell number remains relatively constant with slight decreasing trend over time (p<0.01). Endogenous GFP(−) satellite cells analyzed from single muscle fibers of GFP(+) bone marrow transplant recipients. Satellite cell numbers were assayed 2, 4, and 6 months post-transplant. Average numbers of satellite cells per muscle fiber following radiation were somewhat lesser, but in the same range as those in (A) after 9.6 Gy.
(E) GFP(+) bone marrow-derived satellite cells per fiber remained constant over time (approximately 0.37±0.01 GFP(+) cells/fiber or ca. 5% on average, p>0.5). Differences in average fiber length among groups were not significant and did not contribute to differences in satellite cell number (1698±40 μm, p>0.5). GFP(+) satellite cells per fiber were in good agreement with those in (B).
(F) The numbers of GFP(+) muscle fibers per 100 fibers scored remained essentially constant; the apparent increase over time from two to six months is not significant, but may reflect a trend (p>0.5). Muscle fibers in fixed tissue were analyzed for GFP(+) muscle fibers in 26 transverse-sections to determine the contribution of bone marrow-derived cells to the regeneration of the adult fibers over time. Between 1000-2000 muscle fibers were analyzed at each time point.
(G) Endogenous GFP(−) satellite cells were quantified from single myofibers isolated from exercised and non-exercised-control GFP(+) bone marrow transplant recipients and a 40% decrease in endogenous cell number was evident in the exercised group versus non-exercised (p<0.01). Graphs represent satellite cells counted following 48-60 hours in culture from single myofibers isolated from control and exercised groups, respectively (3 mice per group).
(H) The number of GFP(+) satellite cells per fiber remained essentially the same with or without exercise. However, a non-significant 1.7-fold increase in the exercise group relative to the control group may represent a trend (0.61±0.09 relative to 0.36±0.09 GFP(+) cells/fiber, p>0.5). Differences in average fiber lengths among groups were not significant and did not contribute to differences in satellite cell number (1750±60 μm, p>0.5).
(I) GFP(+) Muscle fibers increase 20-fold analyzed in fixed TA muscle transverse-sections from exercised relative to non-exercised mice (p<0.01). GFP(+) myofibers are indicative of regeneration from GFP(+) satellite cells.
The numerical data is represented in Table 1, bars represent standard error of the mean, and P-values were determined with a students T-test.

FIG. 2. Quantitation of Satellite Cells and Muscle Fibers in Bone Marrow Transplant Recipient and Wild Type Mice. (A) BMDC (GFP+) and endogenous (GFP−) satellite cells were counted after their migration off isolated muscle fibers. (B) GFP(+) muscle fibers were counted in transverse sections of tibialis anterior muscle.

FIG. 3. Confirmation of the Myogenic Phenotype of BMDC Satellite Cells That Migrated Off Isolated Muscle Fibers. After migration off isolated muscle fibers the frequency of myogenic marker expression on satellite cells from bone marrow transplant recipients and wild type mice was determined.

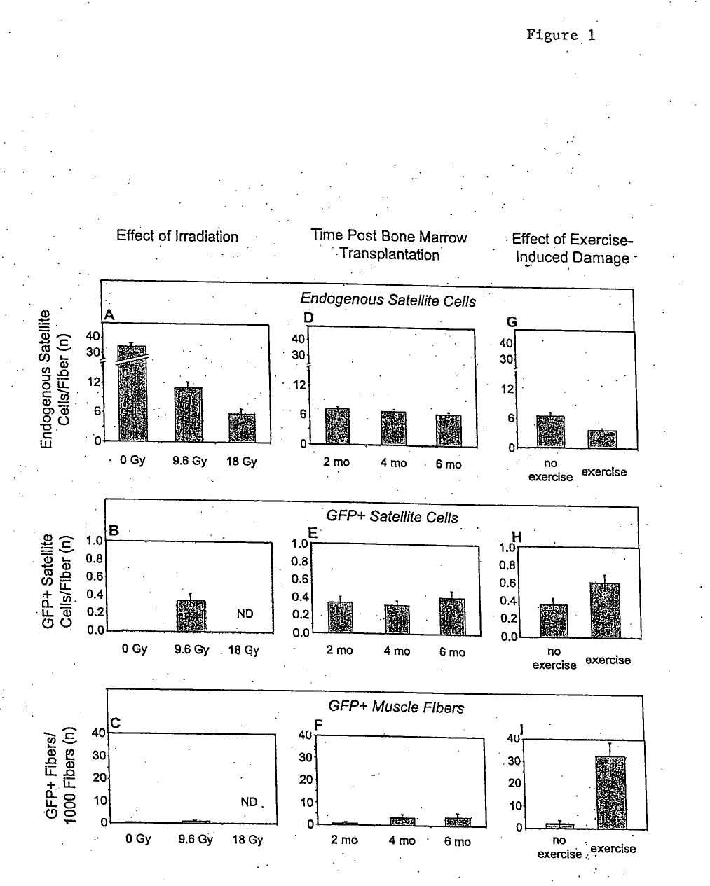

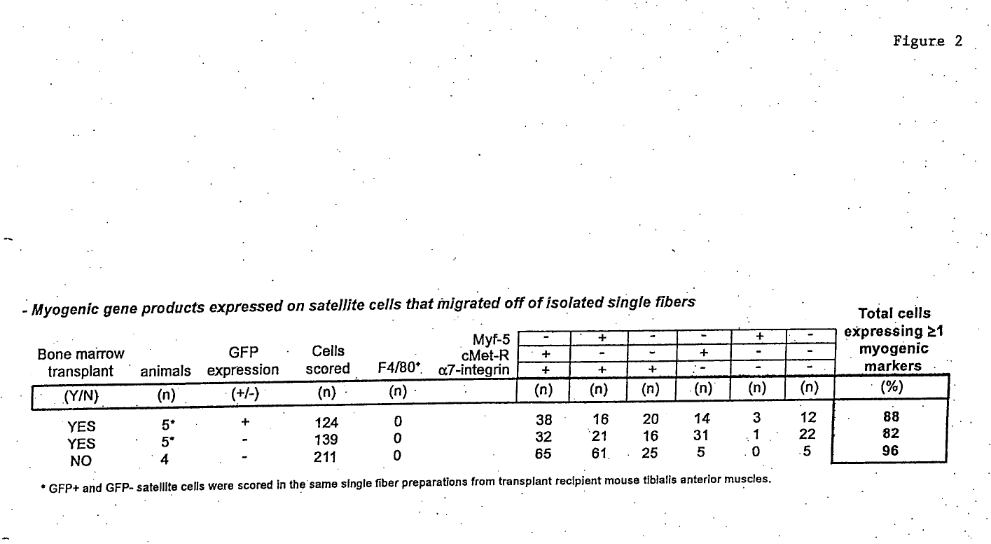

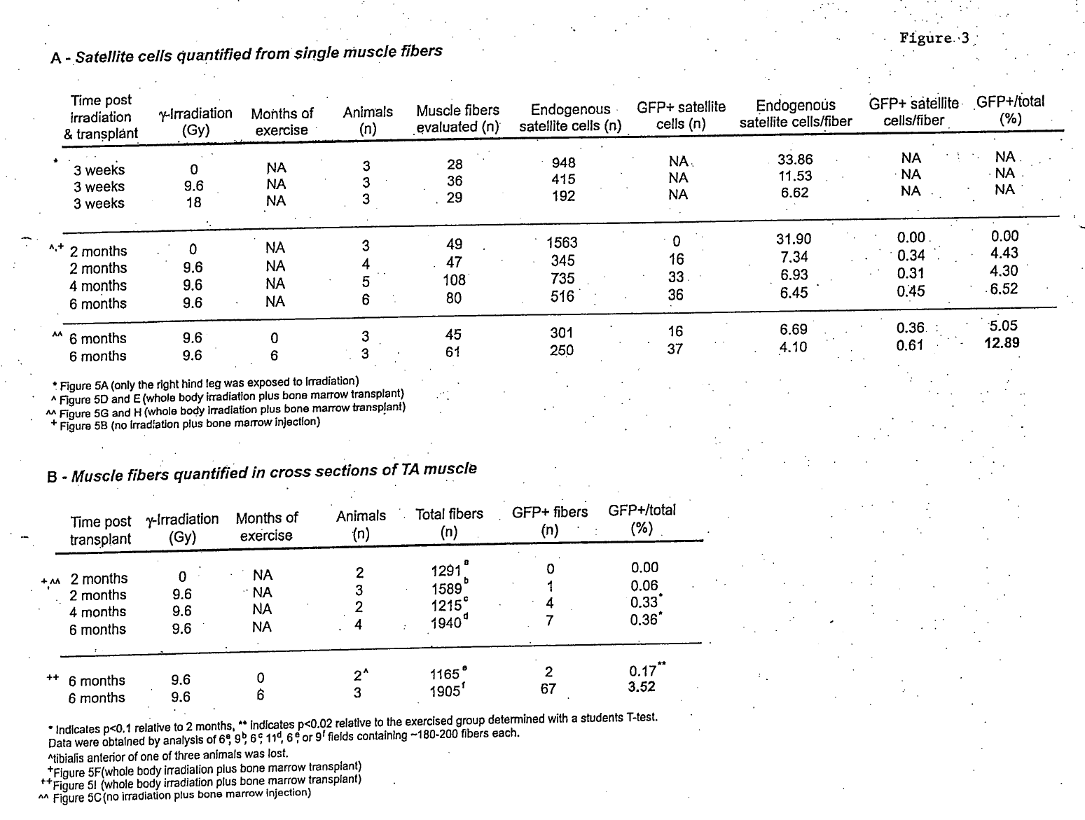

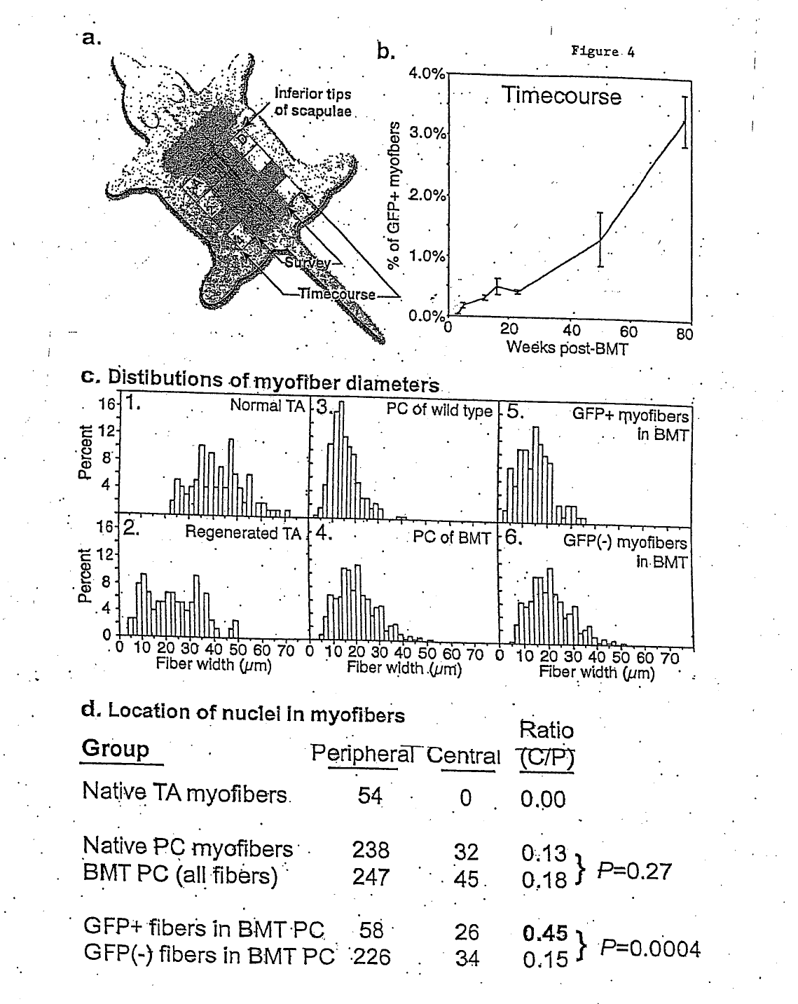

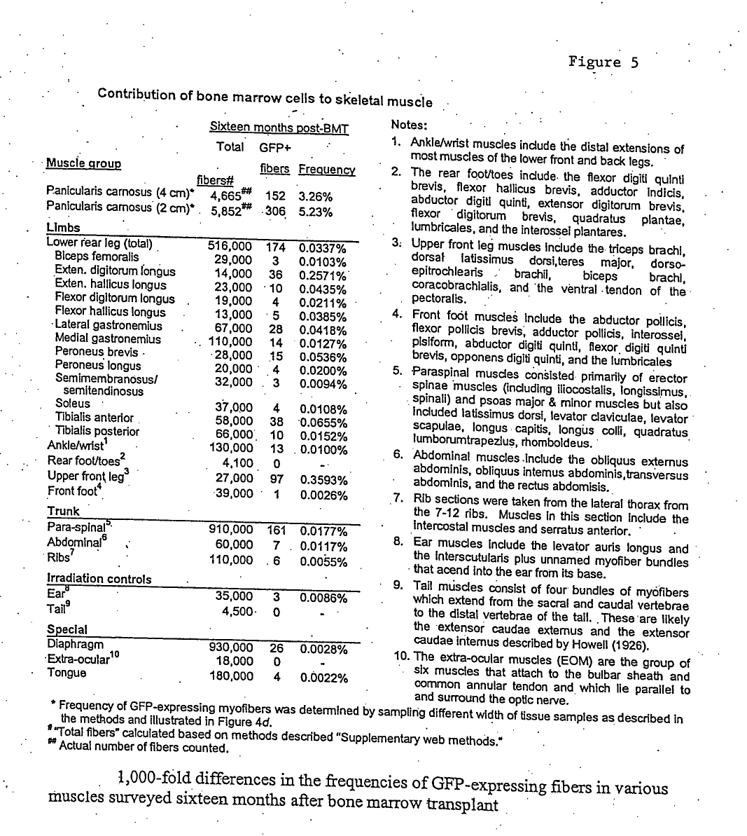

FIG. 4.
(a) Illustration of the two specific areas within the PC with the highest densities of GFP+ myofibers. The first is an approximately 2 cm wide strip centered over and parallel to the lumbar spine and extending from the inferior angle of the scapula to the mid-pelvis. The second area encompassed an approximately 2 cm wide strip, perpendicular to the spine, with the midline of the strip centered over the scapular spines. In this area, the myofibers run at a right angle to the spine and the area of increased GFP+ myofibers extends approximately 3 cm to either side of the spine (in skin mounted between glass). The margins of these areas, while approximate, were consistent in the 24 mice evaluated using whole skin mounts. In areas of the PC outside of these regions, occasional GFP+ myofibers were observed but at a substantially reduced frequency. Samples of the PC were collected by drawing a 4×4 cm square grid centered on the spine (dotted line) with the top row just below the inferior angles of the scapulae. Note that squares 3A and 3B were analyzed for the muscle survey whereas for the time course analysis all four squares in row 3 were analyzed. (b) Time-dependent increase in GFP+ skeletal myofibers in the paniculus camosus continues for over a year after bone marrow transplantation. The reproducibility and consistency of the assay are demonstrated by the relatively small standard deviations for each time point, an essential characteristic for an assay system. (c) Comparative distributions of myofiber sizes demonstrate the heterogeneity in the PC relative to the TA. (c.1 and c.2) The decrease in mean myofiber size and the increase in fiber size heterogeneity in the PC is similar to regenerated (needle track injured) tibialis anterior (TA). (c.3 and c.4) PC myofibers in age-matched wild type and irradiated bone marrow transplanted (BMT) mice are remarkably similar. (c.5 and c.6) The population of GFP-expressing myofibers in the PC exhibits a significant shift toward smaller fiber sizes (P<0.001) relative to non-GFP-expressing myoblasts. These data demonstrate the intrinsic regenerative nature of the PC relative to the TA muscle. (d) Increased incidence of central nucleation following skeletal muscle regeneration. Nuclei in myofibers of the tibialis anterior (TA) of normal mice are located primarily in the periphery whereas an increase in the proportion of centrally located nuclei, characteristic of regenerated skeletal muscle, is observed in the paniculus carnosus (PC) of both normal and bone marrow-transplanted mice. Central nucleation is also significantly increased in GFP+ myofibers compared to non-GFP-expressing myofibers in mice that received a bone marrow transplant from a GFP-expressing donor.

FIG. 5. 1,000-fold differences in the frequencies of GFP-expressing fibers in various muscles surveyed sixteen months after bone marrow transplant.

### DETAILED DESCRIPTION OF THE INVENTION

I. Overview

In certain aspects, the present invention relates to the discovery that endogenous or exogenous bone marrow derived stem cells (BMDSCs) contribute significantly in vivo to various tissues that are not of the traditional hematopoietic lineages. These observations give rise to many practical methods that may be used, for example, in the treatment of disorders and in drug discovery.

One pioneering finding disclosed herein is the discovery that bone marrow derived stem cells infiltrate skeletal muscle tissue, become muscle-specific stem cells (satellite cells) and give rise to mature, differentiated skeletal myocytes, and furthermore, that this process occurs in vivo at rates far higher than previously demonstrated or expected. Tissue-localized stem cells, such as hepatic oval cells and hematopoietic stem cells, have long been recognized as fulfilling a function in replenishing damaged liver and blood, respectively. Remarkably, cells that are not tissue-localized stem cells may be capable of forming, or fusing with, mature cells of various tissues. Even in adulthood, cells within the bone marrow appear to be capable of unexpected differentiation into a variety of mature cell types, although the frequencies of these events in vivo has generally been very low (Korbling et al., 2002; Quaini et al., 2002). Prior to the disclosure presented here, it was unknown whether these changes in cell function resulted from random and rare events or resulted from an endogenous biological process that could be modulated. In certain aspects, methods disclosed herein may be used to promote regeneration or de novo creation of mature or otherwise functional cells of a target tissue in a subject by inducing CSCs to become tissue-localized stem cells in the target tissue, whereby the tissue-localized stem cells produce mature or otherwise functional cells.

Another pioneering finding disclosed herein is the discovery that bone marrow derived stem cells infiltrate neural tissue and fuse with mature neurons to form heterokaryons. This process occurs more frequently in vivo than expected. Although heterokaryons between cells have been generated in vitro, there has not previously been any demonstration that such heterokaryon formation could occur in vivo, or that such heterokaryon formation is a natural process by which stem cells contribute to various tissues. In certain aspects, methods disclosed herein may be used to promote regeneration or maintenance of mature or otherwise functional cells of a target tissue in a subject by inducing CSCs to fuse with cells of the target tissue to form heterokaryons.

Cell fusion has long been known to achieve effective reprogramming of cells in vitro. Almost two decades ago, stable ‘heterokaryons’ were generated by fusing specialized human cells of all three lineages with mouse skeletal-muscle cells to determine whether differentiation is irreversible. These studies demonstrated that muscle genes could be activated in primary human diploid keratinocytes, fibroblasts and hepatocytes by cell fusion. Gene dosage, or the balance of proteins from the two cell types determines which genes are activated in the heterokaryon.

Fusion of a stem cell with a cell of a target tissue in vivo may be exploited to cause changes in the targeted cells. The influx of protein factors from the stem cell may alter gene expression in the resultant heterokaryon. The influx of protein factors may also alter states of a cell that are caused primarily by post-translational regulatory events. For example, proteins from a stem cell may alter the cell cycle progression of a target cell, and may prevent or interfere with apoptotic processes, particularly in neurons where quasi-apoptotic states are known to persist for substantial periods of time. A heterokaryon also receives the cytoplasmic organelles of the fusing stem cell, and accordingly, defects associated with mitochondrial function and other cytoplasmic organelles may be rescued by cell fusion. For example, mitochondrial encephalopathies, such as Leigh's Disease, may be particularly amenable to treatment by stem cell fusion approaches.

Additionally, in vivo cell fusion provides the opportunity for the delivery of heterologous nucleic acids and proteins to the target cells in a form of cell-based gene and protein therapy. For example, fusion of BMDSCs may be used to supply target cells with new genes, such as tumor genes or genes correcting genetic abnormalities. Traditionally, two primary modalities of gene therapy have been proposed: (1) introduction of a nucleic acid encoding a therapeutic gene into a target cell population, generally by viral vector or DNA delivery system; and (2) introduction of cells expressing a therapeutic gene that has non-cell autonomous effects, such as secreted factors. Modality (1) has tended to be limited by the available vectors and erratic, error prone integration of vectors into the target cell. Furthermore, it has been difficult to develop vectors for non-dividing cells. For example, introduction of adenovirus gene therapy vectors caused a fatal liver failure in a human patient. Modality (2) is limited to the types of genes that can be introduced, and introduced transgenic cells may carry activated oncogenes. Cells transfected with gene encoding adenosine deaminase were introduced into children suffering from Severe Combined Immunodeficiency Disease, and although initial results were positive, it soon became apparent that the randomly inserted transgene had caused activation of oncogenes in a small proportion of the introduced cells. The result has been a treatment-resistant leukemia in the gene therapy recipients. By contrast, cell fusion need not result in the integration of the stem cell genome with that of the target cell, and cell fusion, as demonstrated with Purkinje cells herein, is effective on mitotically inactive cells. Additionally, cell fusion may effectively deliver genes encoding proteins that act primarily intracellularly on the heterokaryon.

Accordingly, in certain aspects, the invention provides methods for altering a cell of target tissue by causing fusion of the target cell with a bone marrow derived cell. Optionally, the bone marrow derived cell is genetically altered to contain a desirable transgene, or the cells may be selected so as to have a desirable genotype. A transgene may be expressed from essentially any desired promoter, and in preferred embodiments, the transgene will be expressed from a promoter that causes selective expression in the desired target cell. In certain embodiments, the transgene will be an intracellular protein, such as a pro- or anti-apoptotic signaling protein, a transcription factor, or a protein involved in cell cycle regulation. A fusion mechanism may be particularly preferable for effecting changes in developmentally complex cell types that are difficult to reproduce de novo. For example, complex neurons, such as Purkinje cells are particularly appropriate choices for fusion-based therapy. Diseases involving such cell types include: disorders affecting spinocerebellar regions such as Olivopontocerebellar Atrophy, Friedreich's Ataxia, and Ataxia-Telangiectasia. Additionally, fusion may be particularly effective for treating disorders related to inborn errors of metabolism, including leukodystrophies such as Krabbe's Disease, Metachromatic Leukodystrophy, Pelizaeus-Merzbacher Disease, and Canavan's Disease, and mitochondrial encephalopathies such as Leigh's Disease. Skeletal myocytes are also a complex cell type that may be selected for treatment by a fusion modality. A fusion modality may be most effective in treating disorders characterized by a loss of functional competence, but not outright cell death, in cells of the target tissue. A fusion modality may, however, be effective in preventing, in patients at risk therefore or showing symptomatic progression, diseases that are eventually characterized by death of cells of the target tissue.

Fusion of bone marrow derived cells with cells of a target tissue may be enhanced by any of the various mechanisms disclosed herein for increasing the contribution of a CSC to a target tissue. For example, mobilization, recruitment, survival and maintenance agents may all enhance cell fusion by increasing the likelihood that a CSC is available for cell fusion. Additionally, fusogenic agents may be employed. General fusogens are well known in the art, such as polyethylene glycol and viral fusion proteins, such as HIV Tat. Fusogens may also be identified by screening for such agents. Examples of such screening assays are provided below.

A further pioneering finding presented herein is the discovery that damaged tissues show increased recruitment of bone marrow derived stem cells, thus demonstrating for the first time that the recruitment of pro-regenerative stem cells to tissues can be regulated in vivo and that endogenous, inducible factors regulate the process of stem cell recruitment and tissue regeneration. In certain aspects, the invention provides methods for evaluating an agent that promotes tissue regeneration by testing the effects of such a factor on the contribution of a CSC to a target tissue. Optionally a candidate agent is a factor, particularly a diffusible biomolecule such as a growth factor, produced by cells of a damaged tissue. In certain aspects, the invention provides methods for facilitating stem cell recruitment to a tissue by damaging the tissue. Additional, an embodiment of the invention is the treatment of disorders associated with cellular damage by administration of (a) an exogenous CSC that is recruited to the tissue, (b) an agent that facilitates contribution of an exogenous or endogenous CSC to damaged tissue or (c), a combination of (a) and (b).

Notably, the discoveries and inventions disclosed herein arise in a climate of substantial scientific controversy. Initial reports of the ability of adult, bone marrow-derived cells to contribute to various non-hematopoietic tissues in adults were received by the scientific community with substantial skepticism. Many scientists argued that these results were incorrect and based on experimental artifacts and inadequate methods of cell identification. Several leading stem cell scientists published opinion papers or scientific data in major journals indicating that BMDSC do not have the capacity to generate non-hematopoietic tissues. For example, a paper published in Science by Raymond Castro et al. entitled “Failure of Bone Marrow Cells to Transdifferentiate into Neural Cell in Vivo” (Science 297:1299, 2002) indicated that no cells of hematopoietic origin were present in the CNS of mice (except <5 cells which were associated with blood vessels and presumed to be intravascular). A paper from world-renowned stem cell biologist, Irving Weismann, entitled “Little Evidence for Development Plasticity of Adult Hematopoeitic Stem Cells” suggested that the evidence supporting broad plasticity of hematopoietic stem cells was insufficient and unlikely to be correct. Furthermore, the overall initial pessimism of the scientific community regarding the validity of adult stem cell plasticity has led many leading scientists contend, in review articles, that adult bone marrow-derived cells do not contribute to non-hematopoietic tissues (David Anderson, Fred Gage & Irv Weissman, Can stem cells cross lineage boundaries?, Nature Medicine, 7:393-5, 2001; Jonas Frisén, Stem Cell Plasticity?, Neuron, 35:415-8, 2002; Helen Pearson, Articles of faith adulterated, Nature, 420-734-5, 2002; Brian Vastag, Many Say Adult Stem Cell Reports Overplayed, JAMA 286:293, 2001). Data presented herein have contributed to a resolution of the controversy, with most scientists having now made observations confirming that bone marrow-derived cells do contribute to non-hematopoietic tissues such as neurons, skeletal muscle, and liver.

The disclosure presents a variety of methods by which CSCs may be induced to contribute to peripheral tissues including, for example, increasing the number of exogenous or endogenous CSCs, mobilizing CSCs, stimulating the plasticity of CSCs, stimulating the recruitment and/or incorporation of CSCs into a target tissue, stimulating propagation or maturation of newly formed tissue-localized stem cells, promoting maintenance of tissue-localized stem cells in a target tissue, or a combination of the foregoing. In certain preferred embodiments, a method disclosed herein causes at least about 0.01% of the mature or otherwise functional cells in a target tissue to be derived from tissue-localized stem cells that are, in turn, derived from CSCs that entered the target tissue as a result of the method. In particularly preferred embodiments a method disclosed herein causes at least about 0.1%, 0.5%, 1% or 5% of the mature or otherwise functional cells in the target tissue to derive from tissue-localized stem cells that are, in turn, derived from CSCs that entered the target tissue as a result of the method. In certain preferred embodiments, a method disclosed herein stimulates the production of mature or otherwise functional cells in a target tissue from CSCs by at least about 10-fold the rate seen in the absence of the method, and optionally at least about 100-fold or 1000-fold. In some instances, the rate at which CSCs develop into mature or otherwise functional cells of a target tissue is undetectable unless a method for stimulating this process, such as a method disclosed herein, is employed.

Certain aspects of the invention relate to the use of CSCs as part of regimens in the treatment or prevention of disorders of, or surgical or cosmetic repair of, various target tissues. In certain embodiments, a subject disorder is characterized by a deficiency of mature or otherwise functional cells of a tissue. In certain embodiments, a subject disorder is characterized by a local tissue environment that is not conducive to regeneration, such as a disorder characterized by fibrosis or the presence of necrotic cells. In certain embodiments a target tissue for therapeutic intervention is a tissue having a well-characterized tissue-localized stem cell population, such as neural tissue, skeletal muscle tissue, cardiac muscle tissue, and epithelial tissues, such as respiratory epithelium and skin. In certain embodiments, a target tissue is a connective tissue, such as a cartilageneous tissue (e.g. articular cartilage). In certain embodiments, subject methods can be used for treating atrophy, or wasting, in particular, skeletal muscle atrophy and cardiac muscle atrophy. In a particularly preferred embodiments, methods disclosed herein may be used to treat, prevent or ameliorate muscular disorders associated with normal aging, such as age-related dystrophy. In addition, certain diseases wherein the muscle tissue is damaged, is abnormal or has atrophied, are treatable using the invention, such as, for example, normal aging, disuse atrophy, wasting or cachexia, and various secondary disorders associated with age and the loss of muscle mass, such as hypertension, glucose intolerance and diabetes, dyslipidemia and atherosclerotic cardiovascular disease. The invention also is directed to the treatment of certain cardiac insufficiencies, such as congestive heart failure. The treatment of muscular myopathies such as muscular dystrophies is also embodied in the invention.

Certain aspects of the invention pertain to the use of CSCs for the treatment of other non-hematological or immunological tissues, particularly solid organs. The subject method can be used to repopulate or otherwise increase the population of resident stem cells in the target tissue. In certain embodiments, the subject method includes inducing differentiation of the stem cells in order to generate or repair the tissue of the organ in which the cells are engrafted. The CSCs may be recombinantly engineered to correct one or more genetic defects.

Another aspect of the invention pertains to the use of genetically modified CSCs to produce differentiated cells, in the target tissue(s), which secrete therapeutic moieties, such as proteins or peptides, as a consequence to the genetic manipulation.

Certain methods of the invention have wide applicability for the treatment or prophylaxis of disorders characterized by an insufficiency of functional mature cells of a tissue. Certain methods of the invention may be used to generate or augment tissue, regardless of whether the tissue is disordered or healthy. In general, the method can be characterized as including a step for causing circulating stem cells (“CSCs”) to enter a target tissue and become tissue-localized stem cells.

The subject method has wide applicability to the treatment or prophylaxis of disorders afflicting muscle tissue. In one aspect, the invention can be used for stimulating muscle growth or differentiation. Such stimulation of muscle growth is useful for treating atrophy, or wasting, in particular, skeletal muscle atrophy and cardiac muscle atrophy. In addition, certain diseases wherein the muscle tissue is damaged, is abnormal or has atrophied, are treatable using the invention, such as, for example, normal aging, disuse atrophy, wasting or cachexia, and various secondary disorders associated with age and the loss of muscle mass, such as hypertension, glucose intolerance and diabetes, dyslipidemia and atherosclerotic cardiovascular disease. The treatment of muscular myopathies such as muscular dystrophies is also embodied in the invention.

With denervation or disuse, skeletal muscles undergo rapid atrophy which leads to a profound decrease in size, protein content and contractile strength. This atrophy is an important component of many neuromuscular diseases in humans. In a clinical setting, the methods of the present invention can be used for repairing muscle degeneration, e.g., for decreasing the loss of muscle mass, such as part of a treatment for such muscle wasting disorders.

In certain embodiments, the subject method can be used to treat patients suffering from an abnormal physical condition, disease or pathophysiological condition associated with abnormal and/or aberrant regulation of muscle tissue. For instance, the disorders for which the subject method can be used include those which directly or indirectly produce a wasting (i.e., loss) of muscle mass. These include muscular dystrophies, cardiac cachexia, emphysema, leprosy, malnutrition, osteomalacia, child acute leukemia, AIDS cachexia and cancer cachexia.

The muscular dystrophies are genetic diseases which are characterized by progressive weakness and degeneration of muscle fibers without evidence of neural degeneration. In Duchenne muscular dystrophy (DMD) patients display an average of a 67% reduction in muscle mass, and in myotonic dystrophy, fractional muscle protein synthesis has been shown to be decreased by an average of 28%, without any corresponding decrease in non-muscle protein synthesis (possibly due to impaired end-organ response to anabolic hormones or substrates). Accelerated protein degradation has been demonstrated in the muscles of DMD patients. The subject method can be used as part of a therapeutic strategy for preventing, and in some instance reversing, the muscle wasting conditions associated with such dystrophies.

Severe congestive heart failure (CHF) is characterized by a “cardiac cachexia,” i.e., a muscle protein wasting of both the cardiac and skeletal muscles, with an average 19% body weight decrease. The cardiac cachexia is caused by an increased rate of myofibrillar protein breakdown. The subject method can be used as part of a treatment for cardiac cachexia.

Emphysema is a chronic obstructive pulmonary disease, defined by an enlargement of the air spaces distal to the terminal non-respiratory bronchioles, accompanied by destructive changes of the alveolar walls. Clinical manifestations of reduced pulmonary functioning include coughing, wheezing, recurrent respiratory infections, edema, and functional impairment and shortened life-span. The efflux of tyrosine is increased by 47% in emphysematous patients. Also, whole body leucine flux remains normal, whole-body leucine oxidation is increased, and whole-body protein synthesis is decreased. The result is a decrease in muscle protein synthesis, accompanied by a decrease in whole body protein turnover and skeletal muscle mass. This decrease becomes increasingly evident with disease progression and long term deterioration. The subject method may be used to prevent and/or reverse, the muscle wasting conditions associated with such diseases.

In diabetes mellitus, there is a generalized wasting of small muscle of the hands, which is due to chronic partial denervation (neuropathy). This is most evident and worsens with long term disease progression and severity. The subject method can be used as part of a therapeutic strategy for treatement of diabetes mellitus.

Leprosy is associated with a muscular wasting which occurs between the metacarpals of the thumb and index finger. Severe malnutrition is characterized by, inter alia, severe muscle wasting. The subject method can be used to treat muscle wasting effects of leprosy.

Osteomalacia is a nutritional disorder caused by a deficiency of vitamin D and calcium. It is referred to as “rickets” in children, and “osteomalacia” in adults. It is marked by a softening of the bones (due to impaired mineralization, with excess accumulation of osteoid), pain, tenderness, muscle wasting and weakness, anorexia, and overall weight loss. It can result from malnutrition, repeated pregnancies and lactation (exhausting or depleting vitamin D and calcium stores), and vitamin D resistance. The subject method can be used as part of a therapeutic strategy for treatment of osteomalacia.

In childhood acute leukemia there is protein energy malnutrition which results in skeletal muscle wasting. Studies have shown that some children exhibit the muscle wasting even before diagnosis of the leukemia, with an average 27% decrease in muscle mass. There is also a simultaneous 33%-37% increase in adipose tissue, resulting in no net change in relative body weight and limb circumference. Such patients may be amenable to treatment including the subject method.

Cancer cachexia is a complex syndrome which occurs with variable incidence in patients with solid tumors and hematological malignancies. Clinically, cancer cachexia is manifested as weight loss with massive depletion of both adipose tissue and lean muscle mass, and is one cause of death which results from cancer. Cancer cachexia patients have shorter survival times, and decreased response to chemotherapy. In addition to disorders which produce muscle wasting, other circumstances and conditions appear to be linked in some fashion with a decrease in muscle mass. Such afflictions include muscle wasting due to chronic back pain, advanced age, long term hospitalization due to illness or injury, alcoholism and corticosteroid therapy. The subject method can be used as part of a therapeutic strategy for preventing, and in some instance reversing, the muscle wasting conditions associated with such cancers.

Studies have shown that in severe cases of chronic lower back pain, there is paraspinal muscle wasting. Decreasing paraspinal muscle wasting alleviates pain and improves function. A course of treatment for disorder can include the subject method.

It is also believed that general weakness in old age is due to muscle wasting. As the body ages, an increasing proportion of skeletal muscle is replaced by fibrous tissue. The result is a significant reduction in muscle power, but only a marginal reduction in fat-free mass. The subject method can be used as part of a treatment and preventive strategies for preventing/reversing muscle wasting in elderly patients.

Studies have also shown that in patients suffering injuries or chronic illnesses, and hospitalized for long periods of time, there is long-lasting unilateral muscle wasting, with an average 31% decrease in muscle mass. Studies have also shown that this can be corrected with intensive physiotherapy. However, it may be more effective for many patients to at least augment such therapies with treatment by the subject method

In alcoholics there is wasting of the anterior tibial muscle. This proximal muscle damage is caused by neurogenic damage, namely, impaired glycolytic and phosphorylase enzyme activity. The damage becomes apparent and worsens the longer the duration of the alcohol abuse. Patients treated with corticosteroids experience loss of muscle mass. Such patients may also be amenable to treatment by the subject method.

In certain aspects, methods of this invention can be used for the treatment or prophylaxis of various neurodegenerative diseases and other neural disorders. Cell death has been implicated in a variety of pathological conditions including epilepsy, stroke, ischemia, and neurodegenerative diseases such as Huntington's disease, Parkinson's disease and Alzheimer's disease. Accordingly, CSCs, by becoming tissue-localized stem cells, may provide one means of preventing or replacing the cell loss and associated behavioral abnormalities of these disorders.

Huntington's disease (HD) is an autosomal dominant neurodegenerative disease characterized by progressive movement disorder with psychiatric and cognitive deterioration. HD is associated with a consistent and severe atrophy of the neostriatum which is related to a marked loss of the GABAergic medium-sized spiny projection neurons, the major output neurons of the striatum. Because GABA-ergic neurons are characteristically lost in Huntington's disease, Huntington's patients may be treated by methods disclosed herein. Epilepsy is also associated with neural cell death and may be treated by a methods disclosed herein.

Certain methods of the invention may be used in the treatment of various demyelinating and dysmyelinating disorders, such as Pelizaeus-Merzbacher disease, multiple sclerosis, various leukodystrophies, post-traumatic demyelination, and cerebrovascular (CVS) accidents, as well as various neuritis and neuropathies, particularly of the eye.

Certain methods of the invention may be used for nerve regeneration applications, such as for spinal cord injury repair. The efficacy of a treatment method can be assessed in a rat model for acutely injured spinal cord as described by McDonald et al. (Nat. Med. 5:1410, 1999). A successful treatment will show CSC-derived cells present in the lesion weeks to months later, often differentiated into astrocytes, oligodendrocytes, and/or neurons, and migrating along the cord from the lesioned end. Successfully treated rats should show an improvement in gate, coordination, and weight-bearing.

In certain embodiments, methods of the invention may be used for therapy of a subject in need of having hepatic function restored or supplemented. Human conditions that may be appropriate for such therapy include fulminant hepatic failure, viral hepatitis, drug-induced liver injury, cirrhosis, inherited hepatic insufficiency (such as Wilson's disease, Gilbert's syndrome, or alpha.sub.1-antitryps-in deficiency), hepatobiliary carcinoma and autoimmune liver diseases (such as autoimmune chronic hepatitis or primary biliary cirrhosis). The efficacy of treatment methods can be assessed in animal models for ability to repair liver damage. One such example is damage caused by intraperitoneal injection of D-galactosamine (Dabeva et al., Am. J. Pathol. 143:1606, 1993). Efficacy of treatment can be determined by immunocytochemical staining for liver cell markers, microscopic determination of whether canalicular structures form in growing tissue, and the ability of the treatment to restore synthesis of liver-specific proteins.

In certain embodiments, methods of the invention may be used to repair damaged heart muscle. Heart muscle may be damaged by ischemia (e.g. after infarction) or as a part of the process of heart failure. In addition heart muscle may be damaged by infectious and inflammatory event. The efficacy of a treatment method can be assessed in an animal model for cardiac cryoinjury, which causes 55% of the left ventricular wall tissue to become scar tissue without treatment (Li et al., Ann. Thorac. Surg. 62:654, 1996; Sakai et al., Ann. Thorac. Surg. 8:2074, 1999, Sakai et al., J. Thorac. Cardiovasc. Surg. 118:715, 1999). Successful treatment will reduce the area of the scar, limit scar expansion, and improve heart function as determined by systolic, diastolic, and developed pressure.

In certain embodiments, methods of the invention may be used to treat pancreatic disorders. Autoimmune insulin-dependent (Type 1) diabetes mellitus (IDDM) pathogenesis results from the destruction of the insulin-producing beta cells of the pancreatic islets. Type II diabetes is also responsive, in some individuals, to increased insulin production. Accordingly, certain methods of the invention may be used to generate regeneration of damaged pancreas cells. Insulin production may also be achieved by administering exogenous CSCs that have been genetically modified for improved insulin production, and it may be immaterial whether these cells are located in the pancreas or elsewhere.

In certain embodiments, methods of the invention may be used in the treatment of pulmonary diseases. For example, cystic fibrosis is the most common autosomally inherited disease, and is caused by the defective gene CFTR, which encodes an ion channel at the cell membrane. Augmentation of lung tissue with CSCs may alleviate the reduced respiratory function caused by the defective genotype. As a heritable disorder, this disease is also suitable for treatment CSCs that are genetically altered so as to express CFTR.

In a further embodiment, methods of the invention may be used for the treatment of cartilage damage. Optionally, the cartilage is articular cartilage, and is contained within a mammal and the amount administered is a therapeutically effective amount. Optionally, the cartilage is damaged from a disorder such as osteoarthritis, rheumatoid arthritis, injury, repetitive use or normal aging.

In certain embodiments, a method of increasing CSC contribution to a tissue may further comprise subsequent procedures. For example, it will often be desire to follow any effects of the treatment in the subject. This may be done by, for example testing for improvement in target tissue function. In liver this may be done by testing for a reduction in certain metabolites, such as bilirubins (as distinct from measures of liver damage, e.g. AST, ALT measures, that are routinely made during BMT therapy for cancer patients). In skeletal muscle, this may be done by evaluating muscle strength. Where the target tissue is lung epithelium, tissue function may be evaluated by testing blood gasses, such as O₂ and CO₂. Additionally, contribution of CSCs to target tissues may be evaluated directly by biopsy, at least in those methodologies where cells derived from the CSC can be distinguished from endogenous cells of the target tissue.

In view of this disclosure, other applications for methods of the invention will be apparent to one of skill in the art, especially as additional information about disease etiology becomes available.

II. Definitions

For convenience, certain terms employed in the specification, examples, and appended claims are collected here. Unless defined otherwise, all technical and scientific terms used herein have the same meaning as commonly understood by one of ordinary skill in the art to which this invention belongs.

The articles “a” and “an” are used herein to refer to one or to more than one (i.e., to at least one) of the grammatical object of the article. By way of example, “an element” means one element or more than one element.

“Autologous” implies identical genetic identity between donor cells and those of a recipient patient.

By “DNA” is meant a polymeric form of deoxyribonucleotides (adenine, guanine, thymine, or cytosine) in double-stranded or single-stranded form, either relaxed and supercoiled. This term refers only to the primary and secondary structure of the molecule, and does not limit it to any particular tertiary forms. Thus, this term includes single- and double-stranded DNA found, inter alia, in linear DNA molecules (e.g., restriction fragments), viruses, plasmids, and chromosomes. In discussing the structure of particular DNA molecules, sequences may be described herein according to the normal convention of giving only the sequence in the 5′ to 3′ direction along the nontranscribed strand of DNA (i.e., the strand having the sequence homologous to the mRNA). The term captures molecules that include the four bases adenine, guanine, thymine, or cytosine, as well as molecules that include base analogues which are known in the art.

“Electromagnetic emission” refers to any part of the electromagnetic spectrum that is detected including both visible and invisible emissions. An analysis of the electromagnetic spectrum includes epifluorescent microscopy, confocal microscopy, deconvolution microscopy, other types of microscopy, and the detection or observation of the emission of a fluorophore or visible agent.

A “gene” or “coding sequence” or a sequence which “encodes” a particular protein, is a nucleic acid molecule which is transcribed (in the case of DNA) and translated (in the case of mRNA) into a polypeptide in vitro or in vivo when placed under the control of appropriate regulatory sequences. The boundaries of the gene are determined by a start codon at the 5′ (amino) terminus and a translation stop codon at the 3′ (carboxy) terminus. A gene can include, but is not limited to, cDNA from prokaryotic or eukaryotic mRNA, genomic DNA sequences from prokaryotic or eukaryotic DNA, and even synthetic DNA sequences. A transcription termination sequence will usually be located 3′ to the gene sequence.

The term “heterologous” as it relates to nucleic acid sequences such as gene sequences and control sequences, denotes sequences that are not associated with a particular cell in a manner that such sequences might be found in nature. Merely to illustrate, a “heterologous” gene may be: (a) a gene or coding sequence thereof which has been introduced, such as by homologous recombination, at chromosomal location different from the locus at which that gene normally occurs, (b) a gene or coding sequence thereof located on an episomal vector, (c) a gene having a coding sequence which is operably linked to a transcriptional regulatory sequence which is not normally associated with the coding sequence, (d) a gene having a coding sequence for an artificial (e.g., man-made, non-naturally occurring) protein.

The term “including” is used herein to mean, and is used interchangeably with, the phrase “including but not limited to”.

A “mature cell” is a cell that possesses at least one functional characteristic that is specialized for the tissue in which it is located. Functional characteristics may include metabolic capabilities, morphological characteristics and endocrine or exocrine factor production. In some instances, a mature cell will be capable of self-renewal.

“Multipotent” implies that a cell is capable, through its progeny, of giving rise to several different cell types found in the adult animal.

By “muscle cell” or “muscle tissue” is meant a cell or group of cells derived from muscle, including but not limited to cells and tissue derived from skeletal muscle; smooth muscle, e.g., from the digestive tract, urinary bladder and blood vessels; and cardiac muscle. The term captures muscle cells both in vitro and in vivo. Thus, for example, an isolated cardiomyocyte would constitute a “muscle cell” for purposes of the present invention, as would a muscle cell as it exists in muscle tissue present in a subject in vivo. The term also encompasses both differentiated and nondifferentiated muscle cells, such as myocytes such as myotubes, myoblasts, both dividing and differentiated, cardiomyocytes and cardiomyoblasts.

“Operably linked” refers to an arrangement of elements wherein the components so described are configured so as to perform their usual function. Thus, control elements operably linked to a coding sequence are capable of effecting the expression of the coding sequence. The control elements need not be contiguous with the coding sequence, so long as they function to direct the expression thereof. Thus, for example, intervening untranslated yet transcribed sequences can be present between a promoter sequence and the coding sequence and the promoter sequence can still be considered “operably linked” to the coding sequence.

The term “or” is used herein to mean, and is used interchangeably with, the term “and/or”, unless context clearly indicates otherwise.

“Pluripotent” implies that a cell is capable, through its progeny, of giving rise to all the cell types which comprise the adult animal including the germ cells. Embryonic stem and embryonic germ cells are pluripotent cells under this definition.

The term “polypeptide” as used herein includes compounds having a polypeptide component and a compound of a different chemical nature or bonded through a different type of bond, such as a glycosylation, lipid modification and phosphorylation. The term “polypeptide” is therefore intended to encompass glycoproteins and proteoglycans. The term “polypeptide” also includes polymers comprising one or more unnatural amino acids. Unless context clearly indicates otherwise, the terms “polypeptide” and “protein” are used interchangeably and carry the same meaning.

“Selective analysis” refers to means that allows cells with a specific feature to be analyzed such as a tracking marker or a protein indicative of a cell identity and includes the techniques of flow cytometry, FACS (fluorescence-activated cell sorting), magnetic bead selection or enrichment, affinity column chromatography and immuno-panning.

The term “selective expression” or “selective promoter” refer to a gene expression pattern and the regulatory elements that confer such expression pattern. Selective expression is intended to mean that the gene is expressed at a greater level in the indicated cell types than in other cell types of the target tissue. In some situations, the selectively expressed gene will be widely expressed in other non-target tissues of the body. In other situations, the selectively expressed gene will be expressed at meaningful levels only in the indicated subset of cells of the target tissue. Selective expression may also be used to indicate that a gene is primarily expressed in the cells of a target tissue versus those of other tissues.

“Stem cell” describes cells which are able to regenerate themselves and also to give rise to progenitor cells which ultimately will generate cells developmentally restricted to specific lineages.

A “test agent” is homogeneous or heterogeneous molecular factor that is administered to a subject and includes small molecule factors and polypeptide factors (including polypeptides with chemical modifications or with other molecules attached such as carbohydrate groups). A test agent can also include sugars or carbohydrates, lipid factors, steroid factors, DNA, RNA, growth factors, cytokines, hormones, or chemokines.

A “tissue-localized stem cell” is a cell that is stably associated with a tissue and that gives rise to differentiated cells of that tissue. A tissue-localized stem cell will also be self-renewing. A tissue-localized stem cell may be able to give rise to differentiated cells of one or more other tissues as well, under appropriate conditions.

“Therapeutic protein” refers to a protein which is defective or missing from the subject in question, thus resulting in a disease state or disorder in the subject, or to a protein which confers a benefit to the subject in question, such as an antiviral, antibacterial or antitumor function. A therapeutic protein can also be one which modifies any one of a wide variety of biological functions, such as endocrine, immunological and metabolic functions. Representative therapeutic proteins are discussed more fully below.

The term “transcriptional regulatory elements” refers collectively to one or more of promoter regions, polyadenylation signals, transcription termination sequences, upstream regulatory domains, origins of replication, internal ribosome entry sites (“IRES”), enhancers, and the like, which collectively provide for the replication, transcription and translation of a coding sequence in a recipient cell. Not all of these control elements need always be present so long as the selected coding sequence is capable of being replicated, transcribed and translated in an appropriate host cell.

“Transduction” denotes the delivery of a DNA molecule to a recipient cell either in vivo or in vitro, via a replication-defective viral vector, such as via a recombinant AAV virion.

“Transfection” is used to refer to the uptake of foreign DNA by a mammalian cell. A cell has been “transfected” when exogenous DNA has been introduced inside the cell membrane. The term refers to both stable and transient uptake of the genetic material.

III. Illustrative Embodiments

A. Effects of Damage

As described herein, diverse types of damage resulted in increased contributions of CSC to non-hematopoietic tissues. While the types of damage tested here are diverse and include irradiation, exercise, alloimmune injury, toxin-mediated membrane damage and excitotoxin cell injury (cardiotoxin), and de-inervation-induced myofiber degeneration, (e.g., notexin), they have several features that indicate the classes of regulators that may be used to increase the frequency with which CSC contribute to non-hematopoietic tissue.

A clear feature of all of these injury types is inflammation which ranged from low-level chronic inflammation associated with exercise, modest inflammation associated with irradiation, and substantial inflammation associated with alloimmune injury or toxin-mediated injury. Prominent features of inflammation include the mobilization of cells into the circulation, the homing of inflammatory cells to sites of inflammation, the extravasation of these cells into tissue, the migration of cells to the injury site within a tissue, and the enablement of effector functions of these cells once they arrive. In addition, inflammation also provides the cells involved with proliferative signals as well as maintains an expression of proteins or other signaling factors that are necessary for the maintenance or survival of inflammatory cells.

In addition to inflammation some or all of these injury models induce an angiogenic response, a proliferative response among stromal elements in the injured tissue, remodeling of the extracellular matrix, and elaboration of a variety of growth factors and cytokines by both the cells residing locally within the tissue as well as by infiltrating cells from the circulation. Thus, based on the consistent pro-regenerative response associated with a large number of damage inducing stimuli several classes of molecular agents are anticipated to be important the modulating the frequency with which CSCs contribute to nonhematopoietic tissue.

Inflammation upregulates the expression of VEGF which, in turn, mobilizes endothelial progenitors into the circulation that contribute to inflammation-associated neovascularization. Increased expression of G-CSF at sites of injury has also been reported. Therefore, an agent that promotes movement of BMDSC into circulation is hypothesized to increase the contribution of CSC to non-hematopoietic tissues. Other examples of this include the mobilization of cells of hematopoietic potential into the circulation from the bone marrow compartment following administration of Granulocyte Colony-Stimulating Factor (G-CSF) or the Granulocyte-Macrophage Colony-Stimulating Factor (GM-CSF) induced mobilization of endothelial progenitor cells into the circulation.

Injury is also associated with the production of protein factors that can be characterized as survival factors because their presence at injury sites or in the circulation prevents stressed cells from undergoing apoptotic or other means of cell death. In the absence of these factors a larger proportion of cells at sites of injury are lost. Examples of such factors include HGF/scatter factor in liver regeneration, IGF-1 in skeletal muscle regeneration, and PDGF in mesenchymal cell proliferations such as fibrosis. Therefore, an agent that promotes the survival of bone marrow derived stem cells or CSC in the bone marrow, in circulation, in the target tissue, and/or at the site of injury is hypothesized to increase the contribution of CSC to non-hematopoietic tissues.

Injury and well as inflammation increase the recruitment of cells from the circulation into the damaged tissue in the process called extravasation. For example, mediators frequently found at sites of damage or inflammation such as histamine, thrombin, and platlet-activating factor (PAF) cause adhesion molecules such as P-selectin to be redistributed to the surface of endothelial cells where it can bind leukocytes. Other damage mediators such as IL-1 and TNF (tumor necrosis factor) induce the synthesis and surface expression of other endothelial adhesion molecules such as E-selectin, ICAM-1, and VCAM-1. Therefore, an agent that promotes the recruitment of CSC out of the circulation is hypothesized to increase the contribution of CSC to non-hematopoietic tissues.

The cellular response to damage also includes the movement of cells through tissue to reach the site of damage, a process called chemotaxis. Several substances associated with tissue damage and/or inflammation can act as chemoattractants. Such mediators include components of the complement system such as C5a, products of the lipoxegenase system such as leukotriene B-4 (LTB-4), and cytokines such as those of the IL-8 family. Therefore, an agent that promotes the movement of regenerative or injury-responsive cells within the tissue toward a damaged portion of the target tissue is hypothesized to increase the contribution of CSC to non-hematopoietic tissues.

In order for a cell responding to damage signals to fully activate its effector functions it must receive additional signals from the damaged tissue environment. For example, the production of araachadonic acid metabolites from phospholipids and C5a are potent stimulators of leukocyte activation. PDGF, EGF, TGF-beta, IL-1, and TNF-alpha all activate fibroblasts to proliferation, produce collagen and PGE as well several proteolytic enzymes such as collagenase which contribute to the stromal remodeling that frequently accompanies damage. Therefore, an agent that stimulates or maintains the effector function of regenerative bone marrow-derived stem cells, namely their developmental plasticity, is hypothesized to increase the contribution of CSC to non-hematopoietic tissues.

Additional description of various mechanistic categories of agents that may, in view of the teachings provided herein, be used to modulate CSC contribution to target tissues.

B. Sources of Circulating Stem Cells

In certain embodiments, circulating stem cells are “endogenous” CSCs. The term “endogenous”, as used in reference to CSCs, means that the CSCs are present in the subject and are not supplied from a source external to the subject. In certain embodiments, described below, the invention provides methods for causing endogenous circulating stem cells to enter a target tissue and become tissue-localized stem cells. In certain embodiments, described below, the invention provides methods for causing endogenous circulating stem cells to enter a target tissue and fuse with cells of that tissue. In certain embodiments, the CSC-derived tissue-localized stem cells generate mature cells of the target tissue. The abundance or developmental plasticity of endogenous CSCs may be influenced by the administration of exogenously-supplied factors or by other conditions described herein. Likewise the tendency of endogenous CSCs to enter the target tissue, fuse with cells of the target tissue or become tissue-localized stem cells may be may be influenced by the administration of exogenously-supplied agents or by other conditions described herein. In preferred embodiments, endogenous or exongenous stem cells are BMDSCs, and particularly SPKLS cells (c-Kit⁺Lin⁻Sca1⁺), which are greatly enriched for hematopoietic stem cells. Other types of stem cells are also known to reside in the bone marrow, particularly mesenchymal stem cells that tend to give rise to cells of connective tissues. It is considered that stem cells from locations other than the bone marrow may also move to distal positions in the body and contribute to regeneration of target tissue.

In certain embodiments, CSCs may be provided as “exogenous” CSCs. The term “exogenous”, as used in reference to CSCs, means that the CSCs are administered to the subject. Exogenous CSCs may be autologous (i.e., derived from the same individual) or syngeneic (i.e., derived from a genetically identical individual, such as a syngeneic littermate or an identical twin), although allogeneic CSCs (ie., cells derived from a genetically different individual of the same species) are also contemplated. Although less preferred, xenogeneic (ie., derived from a different species than the recipient) CSCs, such as CSCs from transgenic pigs, may also be administered. When the donor CSCs are xenogeneic, it is preferred that the cells are obtained from an individual of a species within the same order, more preferably the same superfamily or family (e.g. when the recipient is a human, it is preferred that the CSCs are derived from a primate, more preferably a member of the superfamily Hominoidea).

To illustrate, the CSCs can be bone marrow-derived cells (“BMDCs”). BMDCs may be obtained from any stage of development of the donor individual, including prenatal (e.g., embryonic or fetal), infant (e.g., from birth to approximately three years of age in humans), child (e.g. from about three years of age to about 13 years of age in humans), adolescent (e.g., from about 13 years of age to about 18 years of age in humans), young adult (e.g., from about 18 years of age to about 35 years of age in humans), adult (from about 35 years of age to about 55 years of age in humans) or elderly (e.g., from about 55 years and beyond of age in humans).

In some embodiments, the BMDCs are administered as unfractionated bone marrow. Bone marrow may be fractionated to enrich for certain BMDCs prior to administration. Methods of fractionation are well known in the art, and generally involve both positive selection (i.e., retention of cells based on a particular property) and negative selection (i.e., elimination of cells based on a particular property). As will be apparent to one of skill in the art, the particular properties (e.g., surface markers) that are used for positive and negative selection will depend on the species of the donor bone marrow-derived cells.

When the donor bone marrow-derived cells are human, there are a variety of methods for fractionating bone marrow and enriching bone marrow-derived cells. A subpopulation of BMDCs includes cells, such as certain hematopoietic stem cells that express CD34, and/or Thy-1. Depending on the cell population to be obtained, negative selection methods that remove or reduce cells expressing CD3, CDIO, CD11b, CD14, CD16, CD15, CD16, CD19, CD20, CD32, CD45, CD45R/B220, Ly6G, and/or TER-119 may be employed. A preferred enrichment is for cells that are c-Kit⁺, Lin⁻ and/or Sca-1⁺. When the donor BMDCs are not autologous, it is preferred that negative selection be performed on the cell preparation to reduce or eliminate differentiated T cells, thereby reducing the risk of graft versus host disease.

As a further illustrative example, CSCs may be stem cells derived from cultured stem cell lines. A stem cell line will preferably be selected for its ability to give rise to one or more cell types of the desired target tissue (i.e. the desired developmental potential). A stem cell line will preferably be selected for the ability of cells derived from the stem cell line to circulate in the blood stream. In certain instances, the circulatory and developmental properties of a stem cell line will not be known, and steps may be taken to obtain such information. Cells of a stem cell line may be tested in vitro or in vivo for developmental potential. Cells of a stem cell line may also be tested for circulatory ability by, for example, transfecting the cell with a fluorescent marker and, after administering the cells to a test animal (e.g. a mouse or monkey) examining one or more tissues for the presence of the fluorescent cells. Optionally, the tissue to be examined is irradiated prior to administration of the test cells. CSCs may be derived from stem cell lines including embryonic stem cell lines and adult stem cell lines, whether totipotent, pluripotent, multipotent or of lesser developmental capacity. Stem cell lines are preferably derived from mammals, such as rodents (e.g. mouse or rat), primates (e.g. monkeys, chimpanzees or humans), pigs, and ruminants (e.g. cows, sheep and goats). Examples of stem cell lines that may be used as CSCs or tested for use as CSCs include: neural stem cells, mesenchymal stem cells and hematopoietic stem cells. Suitable CSCs may be identified by employing, for example, an assay of the type exemplified in Example 2.

Methods used for selection/enrichment of CSCs may include immunoaffinity technology or density centrifugation methods. Immunoaffinity technology may take a variety of forms, as is well known in the art, but generally utilizes an antibody or antibody derivative in combination with some type of segregation technology. The segregation technology generally results in physical segregation of cells bound by the antibody and cells not bound by the antibody, although in some instances the segregation technology which kills the cells bound by the antibody may be used for negative selection.

Any suitable immunoaffinity technology may be utilized for selection/enrichment of CSCs, including fluorescence-activated cell sorting (FACS), panning, immunomagnetic separation, immunoaffinity chromatography, antibody-mediated complement fixation, immunotoxin, density gradient segregation, and the like. After processing in the immunoaffinity process, the desired cells (the cells bound by the immunoaffinity reagent in the case of positive selection, and cells not bound by the immunoaffinity reagent in the case of negative selection) are collected and either subjected to further rounds of immunoaffinity selection/enrichment, or reserved for administration to the patient.

Immunoaffinity selection/enrichment is typically carried out by incubating a preparation of cells comprising CSCs with an antibody or antibody-derived affinity reagent (e.g., an antibody specific for a given surface marker), then utilizing the bound affinity reagent to select either for or against the cells to which the antibody is bound. The selection process generally involves a physical separation, such as can be accomplished by directing droplets containing single cells into different containers depending on the presence or absence of bound affinity reagent (FACS), by utilizing an antibody bound (directly or indirectly) to a solid phase substrate (panning, immunoaffinity chromatography), or by utilizing a magnetic field to collect the cells which are bound to magnetic particles via the affinity reagent (immunomagnetic separation). Alternately, undesirable cells may be eliminated from the CSC preparation using an affinity reagent which directs a cytotoxic insult to the cells bound by the affinity reagent. The cytotoxic insult may be activated by the affinity reagent (e.g., complement fixation), or may be localized to the target cells by the affinity reagent (e.g., immunotoxin, such as ricin B chain).

In certain embodiments, CSCs are genetically modified. For example, CSCs may be transfected with a nucleic acid construct that drives production of a therapeutic polypeptide or other therapeutic moiety. The therapeutic polypeptide or moiety may contribute directly to the target tissue. For example, a CSC for delivery to cartilagenous tissue may be transfected to promote enhanced collagen production, and a CSC for delivery to the liver may be transfected to express one or more P450 oxidase enzymes. A CSC for delivery to a neural tissue may be transfected to increase production of a neurotransmitter, such as dopamine, serotonin, acetylcholine or gaba-aminobutyric acid. The therapeutic polypeptide or moiety may have a systemic effect or an effect at a distant location. For example, a cell may be transfected to enhance production of a steroid hormone, a prostaglandin, or a clotting factor. As another example, a CSC for delivery to the pancreas of a diabetic patient may be transfected with additional copies of an insulin gene or an insulogenic regulatory factor to promote enhanced production of insulin. Cells that produce a factor with systemic effects, such as insulin, need not localize to a particular target tissue in order to produce the therapeutic polypeptide or therapeutic moiety. The therapeutic polypeptide may be selected so as to specifically complement a genetic defect of a subject. For example, a CSC that produces dystrophin may be introduced into subjects suffering from a dystrophin-related form of muscular dystrophy. Similarly, a CSC that produces the cystic fibrosis transporter (CFTR) may be introduced into subjects suffering from a CFTR-related form of cystic fibrosis. In certain embodiments, a CSC is transfected with a gene encoding an enzyme that catalyzes, or assists in the catalysis of, a reaction to produce a therapeutic moiety.

Introduction of genetic constructs into CSCs can be accomplished using any technology known in the art, including calcium phosphate mediated transfection, electroporation, lipid-mediated transfection, naked DNA incorporation, electrotransfer, and viral (both DNA virus and retrovirus mediated) transfection.

It may be desirable to subject the recipient to an ablative regimen prior to administration of the CSCs. Ablative regimens may involve the use of gamma radiation and/or cytotoxic chemotherapy to reduce or eliminate endogenous CSCs, such as circulating hematopoietic stem cells and precursors. A wide variety of ablative regimens using chemotherapeutic agents are known in the art, including the use of cyclophosphamide as a single agent (50 mg/kg q day×4), cyclophosphamide plus busulfan and the DACE protocol (4 mg decadron, 750 mg/m2 Ara-C, 50 mg/in 2carboplatin, 50 mg/m2 etoposide, q 12 h×4 IV). Additionally, gamma radiation may be used (e.g. 0.8 to 1.5 kGy, midline doses) alone or in combination with chemotherapeutic agents. In accordance with standard practice in the art, when chemotherapeutic agents are administered, it is preferred that the be administered via an intravenous catheter or central venous catheter to avoid adverse affects at the injection site(s).

C. Exemplary Mobilizing Agents

In certain aspects, methods of the invention employ methods for increasing the presence of circulating stem cells in the circulatory system. In certain instances, an increase in the presence of CSCs in the circulatory system improves the incorporation of CSCs into target tissues.

The presence of circulatory stem cells in the circulatory system may be increased by administering exogenous CSCs. Examples of exogenous CSCs are described above. Exogenous CSCs may be administered at any body location that permits the cells to enter the bloodstream, and preferably CSCs are introduced into the circulatory system directly, e.g through venous or arterial injection. Administration may be into the peripheral circulatory system or into the central circulatory system. In certain instances, CSCs may be injected directly into the bone marrow.

In certain embodiments, the presence of CSCs in the circulatory system may be increased by increasing the presence of endogenous CSCs in the blood stream by, for example, administering to the subject an agent that stimulates production of CSCs, an agent that stimulates movement or release of CSCs into the blood or an agent that increases the time that CSCs reside in the blood stream. Examples of agents include granulocyte colony stimulating factor (G-CSF), granulocyte-macrophage colony stimulating factor (GM-CSF), flt3 ligand, IL-6, chemokine GRO-β, AMD-3100 (available from AnorMED, Inc.), interleukin-3 receptor agonist (daniplestim) and functional variants thereof. Certain mobilization agents, such as G-CSF and GM-CSF are known to promote differentiation of certain CSCs at relatively high doses, and may therefore inhibit target tissue colonization if used at too high a dose.

In certain embodiments, the presence of CSCs in the circulatory system may be increased by stimulating the movement of endogenous CSCs, such as bone marrow-derived cells, into the bloodstream. For example, increased mobilization may be achieved by administering to the subject an agent that stimulates mobilization. Examples of agents that stimulate mobilization of BMDCs include and functional variants thereof.

The effectiveness of various methods for increasing the presence of CSCs in the circulatory system may be determined by measuring the CSCs in the blood, e.g. by FACS analysis using labeled antibodies that speicifically bind to diagnostic markers on the surface of the CSCs. Similarly, it is possible to screen for novel agents that increase CSCs in the blood by administering test agents to an experimental subject, such as a mouse or primate, and measuring the CSCs in the blood.

Further examples of factors are listed in section F, below.

D. Exemplary Stability Agents and Maintenance Agents

In certain embodiments, methods of the invention employ agents for maintaining or engaging the plasticity of circulating stem cells. In some instances, some percentage of CSCs in a circulating population of CSCs may not be primed to develop the phenotypic characteristics of a tissue-localized stem cell. In other words, some percentage of CSCs may not be constitutively plastic in their developmental potential, and the number of CSCs that incorporate into a target tissue may be improved by contacting the CSCs with an agent that enhances or engages the plasticity of a greater percentage of the CSCs.

In certain embodiments, methods of the invention employ agents for promoting survival of of circulating stem cells, whether in circulation, in the target tissue or at an injury site in the target tissue.

In certain embodiments, methods of the invention employ methods for stimulating the proliferation, maturation, survival and/or long-term maintenance of tissue-localized stem cells.

In some instances it is desirable to stimulate tissue-localized stem cells to produce mature cells that become a functional part of the target tissue. In certain embodiments, maturation is induced by causing damage or stress to the pre-existing mature cells of the tissue. For example, in muscle, damage may be achieved by exercising the muscle. In liver cells, for example, damage may be achieve by administering a moderate dose of a hepatotoxic substance. While not wishing to be bound to theory, it is expected that damage to mature cells will often stimulate tissue-localized stem cells to generate additional mature cells to replace or augment those that were damaged. This approach may be particularly desirable in instances where the tissue-localized stem cells are derived from genetically modified CSCs.

Satellite cells (SC), the tissue-localized stem cells of skeletal muscle proliferate and differentiate in response to factors such as bFGF, IGF-I, TGF-beta, HGF/scatter factor and PDGF. Satellite cells may be maintained stably in muscle in a quiescent state, and MCAD, alpha-7-integrin may participate in quiescence and may be used for maintenance of quiesence. Activated cells express Myf5 and MyoD and, accordingly, factors that stimulate Myf5 expression may be useful in activation of tissue-localized stem cells of skeletal muscle.

Examples of factors are listed in section F, below.

E. Exemplary Recruitment Agent

In certain aspects, methods of the invention employ agents for stimulating the recruitment of CSCs to a target tissue. For example, an agent may stimulate the movement of cells out of circulation and into a target tissue. An agent may also increase the movement of CSCs within a tissue, particularly towards a site where regeneration will occur (e.g., an injury site). A recruitment agent may also promote the retention of cells at the site where regeneration will occur.

In certain embodiments, the incorporation of CSCs into a target tissue may be stimulated by facilitating movement of cells from the bloodstream into the target tissue. For example, the local or systemic administration of a pro-inflammatory agent effective to promote cell migration out of the circulatory system may be used to facilitate movement of CSCs into the target tissue. Vasodilators such as prostacyclin and nitric oxide (or NO-releasing agents) may be administered at sites near the target tissue where CSC entry is desired to, among other things, open space for migrating CSCs. Chemokines such as IL-8 and monocyte chemotactic protein may be administered at sites near the target tissue where CSC entry is desired to, among other things, stimulate motility of CSCs. Matrix metalloproteases may also be administered to facilitate movement of CSCs out of the circulatory system. In certain embodiments, homing factors may be employed to stimulate migration of CSCs into target tissues.

F. Exemplary Niche Creation Methods

In certain embodiments, a method of the invention employs methods for creating niches for tissue-localized stem cells in the target tissue, or other methods for altering the environment of the target tissue so as to encourage the CSC-dependent regenerative process. The term “niche” is used to indicate a site where a tissue-localized stem cell may reside in the target tissue. Optionally, a niche includes environmental signals that assist in the retention of stem cell characteristics of the occupant cell.

A niche may be created by, for example, eliminating or compromising the regenerative capacity of one or more of the pre-existing tissue-localized stem cells. This approach may be particularly useful when it is desirable to replace endogenous tissue-localized stem cells with exogenous tissue-localized stem cells, especially exogenous cells that have been genetically modified. In certain embodiments, the capacity of tissue-localized stem cells to contribute to the tissue may be compromised by irradiation of the target tissue. The regenerative capacity of pre-existing tissue-localized stem cells may also be decreased by administration of a toxic agent that is specifically targeted to the tissue-localized stem cells. Specific targeting may be achieved by, for example, coupling the toxin to an antibody (or antibody fragment) that specifically binds to a cell surface marker that is selectively expressed the surface of the the tissue-localized stem cells to be eliminated. As demonstrated herein, agents that cause damage to a target tissue will stimulate the contribution of CSCs to the tissue. Examples of types of damage that are useful for this purpose include exercise (particularly in the case of skeletal muscle), specific toxins, such as notexin and cardiotoxin (lethal to muscle fibers) and many specific neurotoxins, as well as general cytotoxins (e.g., inhibitors of oxidative phosphorylation, membrane disrupting agents), irradiation, use or overuse of the target tissue, such as by metabolic or excitatory stimulation (e.g., administering a cardiotoxin to muscle cells or administering a compound that increases the metabolic demands on the liver), induced tissue degeneration involves inducing degeneration as will often occur when a tissue ceases to receive neural impulses (e.g., administering notexin to skeletal muscle) or direct damage to the tissue or cell such as cryoinjury or mechanical injury. Such damage may be calibrated so as to determine the minimum possible damage that will have the desired effect on CSC recruitment.

The pro-regenerative environment of a cell may be improved by a variety of techniques. For example, a pro-regenerative soluble polypeptide factor or extracellular matrix protein may be administered. An agent that decreases or increases the gross inflammation at an injury site may alter the regenerative environment. An agent that decerases fibrosis at an injury site will generally be deisrable, as will a factor that increases vascularization of an injured target tissue.

Niches for tissue-localized stem cells may also be generated by introducing a substance into the target tissue to which the CSCs or tissue-localized stem cells adhere. For example, a tissue may be implanted or injected with certain extracellular matrix components such as proteoglycans or fibronectin. Anionic polymeric hydrogels, such as alginate, may also be used to promote attachment of certain tissue-localized stem cells. Tissue-localized stem cells of skeletal muscle may adhere to an acellular matrix derived from homologous tissue. Tissue-localized stem cells of skeletal muscle may also adhere to surfaces or cells displaying one or more of the adhesion molecules (CAMs) M-Cadherin, N-Cadherin, and N-CAM. Tissue-localized stem cells of neural tissue may adhere to surfaces or cells displaying one or more of NCAM, NRCAM, NgCAM, semaphorins and netrins. CSCs may be induced to incorporate into articular cartilage by attraction to a hydrogel, such as alginate, and optionally an RGD-coated alginate, as well as surfaces or cells displaying a collagen matrix, such as a collagen II matrix. In certain embodiments, a niche may comprise ligands for so-called homing receptors, receptors on the surface of the CSCs that specifically bind to ligands on the surface of niches in target tissues and promote docking of CSCs in the niche. Tissues may be treated to stimulate production of chemotactic agents and/or chemoattractants that assist in the movement of CSCs into appropriate niches within target tissues. Cells engineered to produce such factors may also be introduced into the target tissue to form niches. In certain embodiments, positions in a tissue may have characteristics that are incompatible with stem cell occupancy or with the regenerative capacity of tissue-localized stem cells, and accordingly, removal one or of such characteristics may assist in niche generation. Examples of such characteristics may include characteristics associated with fibrosis and/or post-necrotic state, including, for example, loss of vascularity and connective tissue deposition (in non-connective tissues), such as certain collagens.

In certain embodiments, a tissue retractor is used to generate the artificial space. The retractor selectively moves appropriate tissue out of the way form the space abutting a mesenchymal portion of the tissue or the space in the periosteum. For instance, examples of retractors useful in the methods of the present invention include a fluid-operated portion such as a balloon or bladder to retract tissue, not merely to work in or dilate an existing opening, as for example an angioscope does. The fluid-filled portion of the retractor is flexible and, thus, there are no sharp edges that might injure tissue being moved by the retractor. The soft material of the fluid-filled portion, to an extent desired, conforms to the tissue confines, and the exact pressure can be monitored so as not to damage tissue. In certain embodiments, stents and other barriers can be used to help hold the shape or volume of the expanded area.

In some instances, particularly where the artificial space abuts bone, ultrasonic or other cutting or ablative devices can be used to remove surrounding tissue to permit the expansion of the artificial space.

In certain embodiments, the artificial space is infused with a matrix which is conducive to infiltration by, and growth and/or differentiation of pluripotent cells from the tissue surrounding the artificial space. Suitable matrices have the appropriate chemical and structural attributes to allow the infiltration, proliferation and differentiation of migrating progenitor cells.

In certain embodiments, the matrices are formed of synthetic, biodegradable, bicompatible polymers. The term “bioerodible”, or “biodegradable”, as used herein refers to materials which are enzymatically or chemically degraded in vivo into simpler chemical species. “Biocompatible” refers to materials which do not elicit a strong immunological reaction against the material nor are toxic, and which degrade into non-toxic, non-immunogenic chemical species which are removed from the body by excretion or metabolism.

The organization of the tissue may be regulated by the microstructure of the matrix. Specific pore sizes and structures may be utilized to control the pattern and extent of tissue ingrowth from the host, as well as the organization of the implanted cells. The surface geometry and chemistry of the matrix may be regulated to control the adhesion, organization, and function of implanted cells or host cells. In certain preferred embodiments, the matrix is formed of polymers having a fibrous structure which has sufficient interstitial spacing to allow for free diffusion of nutrients and gases to cells attached to the matrix surface until vascularization and engraftment of new tissue occurs. The interstitial spacing is typically in the range of 50 to 300 microns. As used herein, “fibrous” includes one or more fibers that is entwined with itself, multiple fibers in a woven or non-woven mesh, and sponge like devices.

The support structure is also biocompatible (e.g., not toxic to the infiltrating cells) and, in some cases, the support structure can be biodegradable. The support structure can be shaped either before or after insertion into the artificial space.

In some cases, it is desirable that the support structure be flexible and/or compressible and resilient. In particular, in these cases, the support structure can be deformed as it is implanted, allowing implantation through a small opening in the patient or through a cannula or instrument inserted into a small opening in the patient. After implantation, the support structure expands into its desired shape and orientation.

In certain embodiments, the matrix is a polymer. Examples of polymers which can be used include natural and synthetic polymers, although synthetic polymers are preferred for reproducibility and controlled release kinetics. Synthetic polymers that can be used include bioerodible polymers such as poly(lactide) (PLA), poly(glycolic acid) (PGA), poly(lactide-co-glycolide) (PLGA), and other polyhydroxyacids, poly(caprolactone), polycarbonates, polyamides, polyanhydrides, polyamino acids, polyortho esters, polyacetals, degradable polycyanoacrylates and degradable polyurethanes. Examples of natural polymers include proteins such as albumin, collagen, fibrin, and synthetic polyamino acids, and polysaccharides such as alginate, heparin, glycosaminoglycans (such as hyaluronic acid, chondroitin, chondroitin sulfate, dermatan sulfate, heparin, heparan sulfate, keratosulfate, keratopolysulfate and the like), and other naturally occurring biodegradable polymers of sugar units.

In certain embodiments, the matrix is a composite, e.g., of naturally and non-naturally occurring polymers. To illustrate, the matrix can be a composite of fibrin and artificial polymers.

In certain embodiments, the matrix is a hydrogel. Examples of different hydrogels suitable for practicing this invention, include, but are not limited to: (1) temperature dependent hydrogels that solidify or set at body temperature, e.g., Pluronics™; (2) hydrogels cross-linked by ions, e.g., sodium alginate; (3) hydrogels set by exposure to either visible or ultraviolet light, e.g., polyethylene glycol polylactic acid copolymers with acrylate end groups; and (4) hydrogels that are set or solidified upon a change in pH, e.g., tetronics™.

In still other embodiments, the matrix is an ionic hydrogel. Ionic polysaccharides, such as alginates or chitosan, can be used. In one example, the hydrogel is produced by cross-linking the anionic salt of alginic acid, a carbohydrate polymer isolated from seaweed, with ions, such as calcium cations. The strength of the hydrogel increases with either increasing concentrations of calcium ions or alginate. For example, U.S. Pat. No. 4,352,883 describes the ionic cross-linking of alginate with divalent cations, in water, at room temperature, to form a hydrogel matrix.

All polymers for use in the matrix must meet the mechanical and biochemical parameters necessary to provide adequate support for the cells with subsequent growth and proliferation. The polymers can be characterized with respect to mechanical properties such as tensile strength using an Instron tester, for polymer molecular weight by gel permeation chromatography (GPC), glass transition temperature by differential scanning calorimetry (DSC) and bond structure by infrared (IR) spectroscopy, with respect to toxicology by initial screening tests involving Ames assays and in vitro teratogenicity assays, and implantation studies in animals for immunogenicity, inflammation, release and degradation studies.

Additional factors are listed in section F, below.

G. Exemplary Factors for Modulating Tissue Regeneration by Circulating Stem Cells

In certain embodiments, the incorporation of CSCs into a target tissue may be stimulated by administering one or more of the following: a fibroblast growth factor (“FGF”), a cytokine, leukemia inhibitory factor (LIF), neural growth factor (NGF), ciliary neurotrophic factor (CNTF), growth hormone (GH), erythropoietin (FPO), granulocyte/macrophage colony-stimulating factor (GM-CSF), granulocyte colony-stimulating factor (G-CSF), oncostatin-M (OSM), prolactin (PRI), interleukin (IL)-2, IL-3, IL-4, IL-5-IL-6, IL-7, IL-9, IL-10, and IL-12. Interferons (IFN)-alpha, -beta and -gamma, tumor necrosis factor (TNF)-alpha, nerve growth factor (NGF), platelet factor (PF)4, platelet basic protein (PBP) and macrophage inflammatory protein (MIP)1-alpha and -beta, among others. An FGF is a polypeptide having FGF biological activity, such as binding to FGF receptors, which activity has been used to characterize various FGFs, including, but not limited to acidic FGF, basic FGF, FGF2, Int-2, hst/K-FGF, FGF-5, FGF-6 and KGF. brain-derived neurotrophic factor (BDNF), neurotrophin-3, -4, -5, 4/5 and -6 (NT-3, -4, -5, -4/5, -6), glial-derived neurotrophic factor (GDNF), growth promoting activity (GPA), luteinizing hormone releasing hormone (LHRH), KAL gene (implicated in X-linked Kallman's syndrome, interleukins (e.g., IL-2, IL-6, and the like), platelet derived growth factors (including homodimers and heterodimers of PDGF A, B, and v-sis), retinoic acid (especially all-trans-retinoic acid), epidermal growth factor (EGF), the neuropeptide CGRP, vasoactive intestinal peptide (VIP), gliobastoma-derived T cell suppressor factor (GTSF), transforming growth factor alpha, epidermal growth factor, transforming growth factor betas (including TGF-b1, -b2, -b3, -b4, and -b5), vascular endothelial growth factors (including VEGF-1, -2, -3, -4, and -5), stem cell factor (SCF), neuregulins and neuregulin family members (including neuregulin-1 and heregulin), netrins, galanin, substance P, tyrosine, somatostatin, enkephalin, ephrins, bone morphogenetic protein (BMP) family members (including BMP-1, -2, -3 and -4), semiphorins, glucocorticoids (including dexamethasone), progesterone, putrescine, supplemental serum, extracellular matrix factors (including laminins, fibronectin, collagens, glycoproteins, proteoglycans and lectins), cellular adhesion molecules (including N-CAM, L1, N-cadherin), and neuronal receptor ligands (including receptor agonists, receptor antagonists, peptidomimetic molecules, and antibodies) insulin, insulin-like growth factor-I-alpha, I-beta, and -II (IGF-I-alpha, I-beta, -II), MSX-1 and other regenerative molecules found in an extract of regenerating newt limb. Functional variants of the preceding may also be employed.

H. Exemplary Assays

In certain aspects the invention provides methods for assessing the contribution of CSCs to one or more target tissues. In certain aspects, the invention provides methods for assessing the effects of test agents on the contribution of CSCs to one or more tissues and methods for identifying and/or purifying CSCs that contribute to one or more target tissues. In certain aspects, the invention provides methods for assessing the effects of essentially any variation in conditions on the contribution of CSCs to one or more tissues. In certain aspects, the invention provides methods for assessing the effects of essentially any variation in characteristics of the subject (and/or stem cell donor, where applicable) on the contribution of CSCs to one or more tissues.

In certain embodiments, the invention provides methods for assessing the ability of a test treatment to alter the contribution of a stem cell to a target tissue in a subject, the method comprising: a) administering the test treatment to the subject; b) detecting the contribution of a stem cell to the target tissue; wherein the stem cell is of a distinct developmental lineage from the target tissue. A test treatment may be essentially any desired treatment of the subject, whether intended to increase or decrease stem cell contribution to the target tissue. A test treatment may, for example, comprise administering one or more test agents and/or exposing the subject to one or more conditions (e.g., creating an injury or model disease state in the subject. A preferred subject is a mouse or rat. Contribution of stem cells to the target tissue after treatment may be compared to a reference, which will generally be a measure of contribution in the absence of treatment. A preferred reference is a simultaneous control, optionally a similar, untreated tissue in the same subject.

In instances where the test treatment is administration, the test agent may be essentially any substance, including, for example, a polypeptide, a nucleic acid (e.g., DNA or RNA), carbohydrate, lipid, and a small molecule. An RNA may be an antisense or RNAi probe. Preferred test agents include growth factors, cytokines, hormones and chemokines. For efficiency in screening assays, an agent may be administered with a plurality of additional test agents. This type of pooling allows rapid screening of multiple agents. If an effect is caused by a pool of test agents, smaller subgroups may be tested to identify causative agent(s). Test agents may be administered as appropriate to the assay design. For example, a test agent may be administered at a location expected to provide primarily systemic delivery of the test agent or at a location expected to provide primarily local delivery of the test agent. It should be understood that any administered agent may have some local accumulation and some systemic diffusion. As examples, a test agent may be delivered by a delivery mode selected from among: oral, subcutaneous, transcutaneous and intravenous. Where an agent is a nucleic acid, it may be a nucleic acid that encodes a polypeptide or nucleic acid having some expected effect. A nucleic acid may be delivered naked or in a vector or other delivery complex, so as to be taken up and expressed by cells of the target tissue. A nucleic acid may be within an exogenous stem cell so as to test the effects of the encoded molecule on the stem cell. A nucleic acid may also be placed in a cell for expression, with the cell delivered to the target tissue. A test agent may be selected so as to mimic an aspect of a target tissue damage response. For example, the agent may be a pro-inflammatory agent. An agent may be selected to increase vascularization of the tissue, or to increase infiltration of the tissue by cells in the bloodstream.

A test treatment may also be any sort of condition or other action on the subject. For example, a test treatment may comprise administering one or more of the following to the target tissue: radiation, exercise, a toxin, mechanical damage, cryodamage, damage mediated by immune cells or immune proteins. Preferred toxins include membrane disrupting toxins, excitotoxins (particularly for use with innervated tissues, such as muscle and neurons) and a degenerative toxin. Examples of toxins include notexin and cardiotoxin. A test treatment may also be a change in a physiological or environmental condition, such as an alteration in diet, temperature or intensity and/or frequency of light exposure. A test treatment may also comprise administering exogenous stem cells derived from a donor or subject having one or more of the following criterion: a selected genotype, a selected laboratory animal strain, a selected age and a selected disease state.

In certain embodiments, the invention provides a method for assessing the ability of a test criterion to alter the contribution of a stem cell to a target tissue in a subject, the method comprising: a) detecting the contribution of a stem cell to the target tissue in a subject that has the test criterion; and b) comparing the detected contribution to a reference; wherein the stem cell is of a distinct developmental lineage from the target tissue. A test criterion is generally any feature of a subject (as compared to a control) that is of interest and/or is expected to affect contribution of stem cells to a target tissue. Preferably the stem cell is of a distinct developmental lineage from the target tissue. For example, a stem cell may be a hematopoietic stem cell, and the target tissue may be a target tissue that is not traditionally considered part of the hematopoietic developmental lineage. Examples of test criterion include genotype of subject or stem cell donor, age of the subject or stem cell donor, laboratory animal strain type of the subject, laboratory animal strain type of the stem cell donor, disease state of the subject and disease state of a donor. For example a comparison of mouse strain C57b\6 (Black 6) (having unusual neural stem cell activity) with other more widely used mouse strains may be desirable. Transgenic mice, diabetic mice or mice having abnormalities in the immune or inflammatory systems may be of particular interest. For example, transgenic mice expressing a growth factor of interest may be employed.

In some instances, a stem cell, such as a bone marrow derived stem cell having the tracking marker becomes engrafted in the bone marrow of the subject. A subject may be a transgenic animal comprising bone marrow stem cells having a tracking marker. A donor may likewise be such an animal.

In certain embodiments, a method for assessing the contribution of CSCs to one or more target tissue comprises causing circulating stem cells to have a tracking marker. Preferably a target tissue is a regenerative target tissue. A tracking marker is generally any feature that permits the detection of the administered CSCs, as distinct from cells that were already present in the target tissue(s). Preferred tracking markers are those that are detectable by microscopic techniques, such as fluorescent proteins (e.g. green fluorescent protein and the wavelength-shifted variants thereof), protein or other markers that are detectable with antibodies, chromosomal differences (a “chromosomal feature”), such as cells with a Y chromosome when introduced into a female subject. Tracking markers may be constitutive, or may be turned on ex vivo, prior to administration, or turned on in vivo. Transgenic subject, such as mice, may be designed to turn on tracking markers in response to certain endogenous or exogenous stimuli. Recombinase systems, such as Cre/lox may be used. CSCs may themselves be detected in tissues, as well as differentiated forms, and differentiated form may be progeny or fusion cells (fusions formed between a CSC and a cell of the target tissue), as well as progeny of fusion cells. In certain embodiments, the tracking marker is selected from among the following: a genetically encoded marker and an administered marker. In certain embodiments, a tracking marker may be a genetically encoded marker, selected from among: a reporter gene, a sex chromosome, a chromosomal abnormality, a genetic variation not found in the cells of the subject. A reporter gene may regulated by a tissue- or cell-specific promoter. A marker may also be a dye label or other label that is incorporated into stem cells prior to transplantation and allows tracking of descendants of the labeled stem cells.

Detecting the tracking marker in the target tissue may include detecting the tracking marker in individual cells of the target tissue by, for example, analyzing an electromagnetic emission of the target tissue or a sample thereof (e.g., microscopy, magnetic particle detection). Target tissues may be dissociated to facilitate analysis of particular cells. Separation or enrichment of cell types may be done in culture as well, by selectively allowing outgrowth of certain cell types. In general, selective analysis of one or more selected cell types of the target tissue may be desirable, in order to quantitatively and qualitatively track the contribution of a CSC to a target tissue. For example, it may be desirable to analyze one or more of a mature cell, a tissue-localized stem cell, a cell of the parenchyma, a cell of the stroma (e.g., a fibroblast). Examples of cell types include: a neuron, a Purkinje cell, a muscle stem cell, a skeletal myocyte, a cardiomyocyte and a fibroblast. Other cell types from different target tissues may be selected. A rapid method for cell analysis is cell sorting, particularly FACS. Other types of cell sorting include affinity methods (e.g., adhesion to marker-binding antibodies).

Preferred target tissues include neural tissue, skeletal muscle tissue, heart muscle tissue, pancreatic tissue, cartilaginous tissue, adipose tissue and epithelial tissue, such as gastrointestinal epithelium, lung or airway epithelium, and epithelium of an endocrine and/or exocrine organ.

Optionally, a subject is prepared to enhance CSC incorporation. Tissue may be prepared by exposing the tissue to a condition that decreases the regenerative capacity of one or more tissue-localized stem cells in one or more target tissues. Alternatively, conditions may damage target tissue so as to promote regenerative processes. The conditions may be targeted at certain tissues, or the subject as a whole may be exposed to the conditions. Conditions may include, for example, irradiation and targeted ablation, as described in the section pertaining to niche creation. It is also contemplated that niche creation methods, as described above, may be used to prepare the target tissue. For example, a niche for CSCs may be created in the target tissue by introducing an appropriate matrix. CSCs are administered to the subject, and the presence of the CSCs (or progeny or fusions thereof) in the target tissue(s) is detected, and optionally quantified. The CSCs may be selected or designed to have a detectable feature. Target tissues are preferably one or more skeletal muscles, such as the paniculus carnosus (PC), but other target tissue types, including, for example, skin, brain, heart muscle and cartilaginous tissues are contemplated. In certain embodiments, an assay of the invention for assessing the effects of test compounds comprises: (a) causing endogenous or exogenous circulating stem cells to have a tracking marker; (b) administering the test agent to the subject; (c) detecting the presence of the marked circulating stem cells or progeny or fusion cells derived therefrom in one or more regenerative target tissues. The test agent may be administered before, after or simultaneous with part (a). In certain embodiments, methods disclosed herein permit CSCs to form at least about 0.01% of the cells of the target tissue, optionally at least about 0.05%, 0.1%, 0.5%, 1.0% or at least about 5% of the cells of the target tissue. A higher percentage is desirable in certain embodiments because detection may be done more rapidly and/or in fewer test subjects. In certain embodiments, methods disclosed herein permit detection of CSCs (or cells derived therefrom) in target tissues by about one, two, four, ten, or twenty weeks. A more rapid ability to detect may be desirable to facilitate more rapid identification of test agents, or in other circumstances that, in view of this specification will be apparent to one of skill in the art.

In certain embodiments, a method of the invention may be used to assess the ability of a test agent to affect the ability of CSCs to contribute to target tissue(s). An assay may be performed as described above for assessing the contribution of CSCs to target tissue(s), with the addition of administration of a test agent. In general, the test agent is administered at a point in the assay when it is expected that the test agent will have an effect. For example, a test agent may be administered after or just before administration of the CSCs. Certain agents with long acting effects, such as certain steroid hormones, may be administered well in advance of the CSCs. Optionally, the test agent may be situated in the target tissue. A test agent may be essentially any substance of interest, and optionally the test agent is a polypeptide, a small molecule, a nucleic acid, or a natural product. The contribution of CSCs to target tissue(s) in the presence of the test agent may be compared to a suitable reference. An example of a suitable reference is a reference subject that has been treated essentially identically except that the test subject has not received the test agent. A reference subject may be treated at the same time as the test subject, or earlier or later in time. Optionally, a suitable reference is an average obtained from a plurality of reference subjects. Optionally, the reference subject is a subject that has been treated with a different test agent or an agent with known effects. Assays of this type may be used to screen one or a number of test agents, and assays of this type may also be used to optimize or otherwise characterize a test agent already known to have some effect on the contribution of the CSCs to the target tissue(s).

In certain embodiments, a method of the invention may be to identifying or enrich for a circulating stem cell that contributes to one or more target tissues in a subject. In certain embodiments, a subject is exposed to a preparatory condition, such as a condition that kills one or more tissue-localized stem cells in one or more of the target tissues, or another niche-creating technique. A test cell population is administered to the subject, and the contribution of the test cells to the target tissue(s) is detected. Optionally the test cells are designed or selected to have a detectable feature. If cells of the test cell population contribute to the target tissue(s), then it may be inferred that the test cell population comprises circulating stem cells appropriate for the target tissue(s). Assays of this type may be used to assess the presence of CSCs in various cell fractions. For example, bone marrow cell suspensions may be fractionated by, for example, fluorescence activated cell sorting, and the different fractions assessed for ability to contribute to target tissue(s).

A method for evaluating the effect of a treatment or criterion on stem cell contribution to a tissue may be followed by additional tests to further evaluate the ability of the test condition or criterion to affect regeneration of a damaged tissue in a subject. For example, tests may be conducted in a disease model animal or an animal with target tissue damage to assess improvements or degradation as a result of the test treatment or criterion. For test agents, it may be desirable to perform additional testing the relationship between the dosage level of the test agent and the level of contribution of stem cells to the target tissue (i.e. dose response curve). Other subsequent tests, particularly those involved in drug development, will, in view of this disclosure, be apparent to those of skill in the art.

Assays disclosed herein may be performed on a variety of animals, including mice and rats, but also including human volunteers (where safe and appropriate), non-human primates, guinea pigs, rabbits, chickens, frogs and the like.

Accordingly, methods are provided herein for identifying a variety of CSCs and for identifying a variety of substances that affect the ability of CSCs to contribute to target tissue(s).

### EXAMPLES

### Example 1

### Biological Progression from Adult Bone Marrow to Mononucleate Muscle Stem Cell to Multinucleate Muscle Fiber

To determine, among other things, whether the contribution of BMDC to tissues is biologically relevant, Applicants examined whether BMDC could become mononucleate diploid heritable stem cells en route to becoming multinucleate differentiated myofibers. Specifically, Applicants tested the hypothesis that in mice, a progression from adult bone marrow to adult muscle fibers occurs via a tissue localized stem cell intermediate, the quiescent muscle satellite cell. Tissue-localized stem cells occupy niches, microenvironments that instruct and support stem cell self-renewal, proliferation and differentiation (Schofield, 1978), providing specific cellular neighbors, signaling molecules, and extracellular matrix components (Spradling, 2001; Watt and Hogan, 2000). It is well known that in response to a stress-inducing injury, endogenous satellite cells contribute to mature muscle fibers at a relatively high frequency (Grounds, 1999), and that injected muscle cell precursors can replace endogenous satellite cells ablated by gamma irradiation (Blayeri et al., 1999). Muscle stem cells, known as satellite cells, are well defined anatomically and biochemically, both in vivo and in vitro (Cornelison and Wold, 1997; Mauro, 1961; Zammit and Beauchamp, 2001). In this report, Applicants demonstrate that following bone marrow transplantation, cells from the bone marrow respond to two temporally distinct biological cues. First, irradiation-induced damage, which leads to ablation of endogenous satellite cells in the muscle stem cell niche, resulted in occupancy of this niche by BMDC. Second, subsequent exercise-induced damage caused BMDC satellite cells to participate in the regeneration of multinucleate muscle fibers at a frequency (3.5%) significantly greater than previously reported for any bone marrow to muscle conversion. The bone marrow-derived cells became heritably myogenic. As satellite cells they expressed muscle-specific proteins in vivo and in vitro, exhibited self-renewal in tissue culture, giving rise to proliferative clones of myoblasts. These myoblast progeny could differentiate to form myotubes in culture or fuse with host myofibers following injection into muscle tissues of mice. Clones derived from single BMDC myoblasts expressed GFP as well as the muscle markers desmin, Myf-5, cMet-R, and α7-integrin, on a par with control primary myoblasts. Together, these data demonstrate that in adult mice, bone marrow derived cells give rise to tissue-localized karyotypically diploid stem cells, muscle satellite cells, both anatomically and functionally and that these cells can proliferate as myoblasts and participate significantly in normal regenerative processes in response to two temporally distinct injuries.

Characterization of Muscle Stem Cells, the Satellite Cells

Muscle stem cells, or satellite cells, can be visualized by microscopy as mononucleate cells located between the plasma membrane and the basal lamina that ensheathes each myofiber (Mauro, 1961). Intact single muscle fibers were isolated, on which the closely juxtaposed satellite cells can be readily visualized in tissue culture. To isolate individual fibers, the tibialis anterior muscle (TA) from the legs of mice was dissociated and the single fibers isolated with a Pasteur pipet following trituration. Isolated fibers were then cultured overnight, a time-period which allowed activation of the transcription factor Myf-5, yet did not induce proliferation of satellite cells or their migration from the fiber (Rosenblatt et al., 1995). Either individual thin optical sections or three dimensional (3-D) reconstructions of serial optical sections of these fibers were analyzed using a laser scanning confocal microscope and antibodies to characteristic proteins. This rigorous analytic method ensures that the colocalization of markers represents true co-expression of different proteins within the same cell (Brazelton et al., 2000; Komack and Rakic, 2001).

Expression of Myf-5 was observed, Myf-5 is the earliest expressed of a family of bHLH transcription factors in muscle, a factor critical to initiating the myogenic program in satellite cells (Cossu et al., 1996). The other two show cMet-R, a tyrosine kinase receptor that is a well accepted marker of satellite cells (Cornelison and Wold, 1997). In each case, nuclei were stained and expression of α7β1 integrin (α7-integrin) in the membranes surrounding both the myofiber and satellite cells are shown (Bao et al., 1993). α7-integrin is readily apparent on the surface of satellite cells. 3-D reconstructions of optical sections collected with a laser scanning confocal microscope showed the satellite cells on the upper surface and sides of the myofibers. Single optical sections through single satellite cells were also obtained. In all four fields, the satellite cells juxtaposed to the muscle fibers have the characteristic high ratio of nucleus to cytoplasm and the nucleus appears to occupy most of the space circumscribed by the α7-labeled or cMet-R-labeled satellite cell membrane. As shown here the membrane protein, α7-integrin, serves as a useful adjunct to the routinely used cMet-R and Myf-5, as it allows visualization of satellite cells in the context of intact individually isolated myofibers.

Progression of Bone Marrow to a Tissue-Localized Stem Cell

Applicants designed experiments to test the hypothesis that BMDC could give rise to satellite cells, mononucleate muscle-specific stem cells. Although several recent reports have shown that BMDC can contribute to mature adult multinucleate skeletal myofibers in bone marrow transplant recipients (Bittner et al., 1999; Ferrari et al., 1998; Ferrari et al., 2001; Gussoni et al., 1999), given the unexpected nature of these findings and their low frequency (ca. 0.2% of fibers), questions have been raised regarding their biological relevance (Anderson et al., 2001). Applicants designed experiments to determine if BMDC transplanted from mice transgenic for GFP could replenish some of the satellite cells depleted following irradiation. 10-week-old syngeneic mice received 9.6 Gy whole body irradiation followed by transplantation via tail vein injection of 10⁶ GFP-labeled (GFP(+)) bone marrow cells from age-matched donors. Mice were sacrificed 2-6 months post transplantation, the TA muscles were dissected, and single isolated myofibers analyzed by confocal scanning microscopy in conjunction with immunohistochemistry. 3-D reconstructions were derived from a composite of twenty optical sections obtained by laser scanning confocal microscopy of single isolated fibers of the TA. GFP(+) satellite cell nuclei expressing Myf-5 were observed. GFP(+) satellite cells were also observed separated by α7-integrin from its adjacent myofiber. All satellite cells have nuclei stained with ToPro and are circumscribed by α7-labeled membranes. Higher magnification of satellite cells clearly shows the colocalization of GFP, nuclear ToPro and the satellite cell marker, cMet-R on intact myofibers in vivo in muscle tissue. These data demonstrate that following a bone marrow transplant, GFP(+) cells from the bone marrow can gain access to and occupy the satellite cell niche, as shown in fibers either isolated in culture or present in intact muscles.

BMDC Muscle Stem Cells Exhibit a Heritable Change in Cell Phenotype

Applicants tested whether bone marrow-derived GFP(+) satellite cells had undergone a heritable change, were stably myogenic, and capable of self-renewal and differentiation as myotubes in culture. Myoblasts, derived from satellite cells (Zammit and Beauchamp, 2001), were isolated by dissociating the muscle tissues from four different GFP(+) bone marrow transplant recipients, as previously described (Rando and Blau, 1994). These cells were sorted twice by FACS and gated so that >99% of the cells collected were GFP(+) and therefore derived from bone marrow and expressed the muscle protein α7-integrin.

To examine heritability of the myogenic phenotype of bone marrow-derived muscle stem cells, FACS-sorted cells were plated at limiting dilution (0.1 cells/well) to ensure clonality and grown in 96-well plates. The changes in gene expression persisted in their progeny. Clones derived from single satellite cells show coincident expression of the bone marrow marker GFP and the muscle specific intermediate filament protein desmin. Single cells within clones also expressed cMet-R, Myf-5, and α7-integrin. These data show that the reprogramming of BMDC to a muscle-specific stem cell entails a heritable change that is passed on to myogenic progeny upon cell division.

An issue of major interest is whether changes in cell fate arise due to cell fusion or to activation of previously silent genes. To address this question, applicants analyzed the karyotype of the cells isolated by FACS. To this end, muscle tissue was dissociated and plated for only 3.5 or 5.5 days to allow cells to adhere to tissue culture plates. Minimal cell division occurs during this period. Cells from bone marrow transplanted mice and from wild type controls were then FACS-sorted and exposed to the microtubule inhibitor, nocodozole, overnight. Following fixation, the metaphase chromosomes of cells were counted to reveal their karyotype. Virtually all were diploid (2N).

To determine whether the clones derived from single cells could differentiate, they were exposed to low mitogen media. When pools of myoblasts were exposed to differentiation medium, multinucleate myotubes that expressed desmin and the bone marrow-marker GFP were evident. Moreover, 13 clones derived from single cells had myotubes ranging in size from 3 to 10 nuclei.

To determine whether BMDC myoblasts could participate in myogenesis in vivo in mice, approximately 10⁵ FACS-sorted bone marrow-derived myoblasts that were both GFP(+) and α7-integrin+ were injected into the TA muscles of 6 SCID mice. Seven days later the muscles were assayed histologically in tissue sections. GFP(+) fibers were detected in transverse sections (10 μm thick) from each of the mice. Moreover, the same GFP fiber could be detected in sections separated by 200 μm, showing that the cells had contributed to intact fibers similar to their unlabeled neighbors.

In summary, these results show that BMDC can adopt functions characteristic of muscle stem cells. They are diploid and assume an anatomical position either in isolated fibers or in fibers in intact muscle tissues consistent with satellite cells. They grow as clones expressing myogenic markers showing that their change in gene expression is heritable. When exposed to low mitogen medium, they fuse like cloned primary myoblasts in tissue culture and when injected into muscle in mice, they are incorporated into myofibers. Thus, by all of these criteria they can be considered muscle-specific stem cells, or satellite cells.

Effect of Irradiation on GFP+ Satellite Cell Number

It has been well established that irradiation dramatically depletes the muscle stem cell number in the TA (Heslop et al., 2000). To determine whether BMDC could replace some of these lost damaged cells, the following experiments were performed.

First, applicants sought conditions that would replicate the reported loss in endogenous satellite cells after irradiation. The effects of the 9.6 Gy used routinely for lethal irradiation prior to bone marrow transplantation in all of the experiments described above was compared with the 18 Gy reported previously to deplete satellite cells in muscle. The mice were shielded in a lead-jig such that only their right-hind limb was exposed to the irradiation source. Three weeks post-irradiation, satellite cells were counted from a total of 93 muscle fibers of similar length (1600±60 μm, p>0.5) isolated from the dissociated TA muscles of 6 mice. Isolated fibers from both right (irradiated) and left (non-irradiated control) legs of each mouse were analyzed. These single fibers were cultured in individual wells for 48-60 hours in conditions that permit migration of satellite cells away from the fiber yet minimize proliferation (Rosenblatt et al., 1995). The results of these studies showed that by comparison with non-irradiated control legs (0 Gy), a marked decline in the number of satellite cells per fiber, from 33±5 to 11±1 and 6±1, was observed after exposure to 0 Gy, 9.6 Gy and 18 Gy, respectively. With 9.6 Gy the reduction in endogenous satellite cells per fiber approximated 80% when determined 2-6 months post transplant, and this value remained constant over time (p>0.5) at 6.9±0.3 satellite cells per fiber.

Applicants then determined whether the marked depletion by irradiation of the endogenous satellite cells was sufficient to open a niche that BMDC could enter. Whole body irradiated and non-irradiated (control) GFP(+) bone marrow transplant recipients were sacrificed and compared 2 months post-transplant. Single fibers were isolated, cultured, and their associated satellite cells counted as described above. In the absence of irradiation, no GFP(+) satellite cells were detected, whereas there were on average 0.37±0.1 GFP(+) satellite cells per fiber, thus 5% of the remaining satellite cells post-irradiation were GFP(+) satellite cells, a number that remained constant 2-6 months after transplantation (p>0.5). The GFP(+) and GFP(−) satellite cells that migrated from single isolated fibers were also characterized in culture with respect to their expression of myogenic markers. Fibers from 5 bone marrow transplanted and 4 wild type control mice were assayed by immunocytochemistry for the muscle proteins, cMet-R, Myf-5, and α7-integrin. Frequency of expression of these three markers were similar to wild type satellite cells and greater than 88% of the GFP(+) cells expressed one or more markers (Table 2). These data show that the vast majority of the GFP(+) cells that migrate from isolated intact fibers are myogenic and that their frequency of myogenic marker expression is on a par with non-transplanted controls.

Effect of Irradiation on GFP(+) Muscle Fibers

To examine the GFP(+) satellite cell contribution to muscle fibers after irradiation, serial 10 μm thick transverse sections of fixed TA muscles were analyzed by laser scanning confocal microscopy in which on average 200 fibers could be visualized and scored per field. Unlike satellite cells, muscle fibers can be readily identified in transverse sections of adult muscle tissue. By contrast with satellite cells, only 1 mature GFP(+) muscle fiber was observed two months post transplant among the 1589 fibers analyzed in irradiated transplant recipient mice. These results indicate that although the GFP(+) satellite cells can contribute to muscle fibers post-irradiation, they do so at an extremely low frequency, less than 1% (Ferrari et al., 2001; Gussoni et al., 1999). Not surprisingly, the TA muscles of the non-irradiated transplant recipients had no detectable GFP(+) muscle fibers. The number of fields and total muscle fibers scored 2, 4, and 6 months post transplant are shown in the figures and no significant difference was observed over time. Taken together, these data suggest that a certain proportion of the satellite cell pool is regenerated within a short time period following irradiation treatment and that the GFP(+) marrow-derived satellite cells, like endogenous satellite cells, occupy this niche and persist over time in a quiescent state with minimal contribution to muscle fibers.

Effect of Exercise-Induced Damage on GFP(+) Satellite Cells

Continuous exercise is thought to cause damage to intracellular and membrane components of the muscle fibers due to the intense shearing forces. In response to cues resulting from muscle damage, wild type mononucleate satellite cells become mitotic, fuse into, and actively participate in the regeneration of the injured muscle tissue resulting in hypertrophic multinucleate fibers. To determine whether marrow-derived GFP(+) cells not only appeared to be satellite cells based on morphological criteria and their behavior in tissue culture, applicants tested whether they could also function as satellite cells in response to exercise-induced damage.

The following experimental protocol was used. One week following a GFP(+) bone marrow transplant, three mice were given a running wheel to allow voluntary exercise and compared with three controls that did not exercise in this manner for a 6-month period. At the time of sacrifice, both TA muscles of each of the 6 mice were dissected. From each mouse, one TA muscle was used to isolate single fibers and the numbers of GFP(−) endogenous satellite cells and GFP(+) donor-derived satellite cells were quantified as described above. The other TA muscle was fixed and cross-sectioned to quantify fiber numbers in the muscle. GFP-expression in a fiber served to indicate that bone marrow-derived GFP(+) satellite cells had responded to signals released in response to exercise-induced injury and regenerated damaged muscle fibers.

Overall satellite cell numbers per fiber (GFP+ or GFP−) did not change markedly in response to exercise. Endogenous GFP(−) cells were reduced somewhat. Whereas a slight increase in the number of GFP(+) satellite cells per muscle fiber was observed following exercise.

Effect of Exercise-Induced Damage on GFP(+) Muscle Fibers

By contrast with satellite cells, exercise-induced damage resulted in a marked increase of 20-fold GFP(+) myofibers after six months exposure to a running wheel (from 0.16 to 3.52 GFP+ myofibers/100 myofibers). This contribution to muscle fibers of GFP(+) satellite cells was determined by scoring the number of GFP(+) myofibers in transverse-sections of the fixed TA muscle. The 20-fold difference reflects the proportion of GFP(+) myofibers in the group that did not exercise relative to the group that did, 0.17% relative to 3.52% of total fibers analyzed (1165 and 1905 muscle fibers respectively) (Table 1B). Moreover, although the GFP(+) muscle fibers were sometimes dispersed they were often observed in clusters, suggesting that bone marrow-derived regeneration may have originated from single satellite cell clones (Hughes and Blau, 1990).

In summary, these data show that irradiation has a major impact on the incorporation of GFP(+) marrow derived cells into the satellite cell (stem cell) compartment, whereas exercise has a major impact on the incorporation of these cells into muscle fibers. Moreover, contribution of bone marrow cells to these two steps in the biological progression typical of muscle can be dissociated using two different sequential damage-inducing treatments.

Biological Progression in Response to Temporally Distinct Injury-Induced Signals

By separating the transit from blood to muscle into two phases applicants were able to provide evidence that the contribution of cells within bone marrow to a specialized non-hematopoietic tissue, follows a biological progression in response to biological stimuli. Applicants first showed that genetically marked GFP(+) bone marrow-derived cells (BMDC) occupy the muscle-specific stem cell niche following depletion by radiation of the endogenous satellite cells. These cells were heritably altered, were capable of self-renewal as myogenic clones, generated multinucleate muscle cells in tissue culture in response to media that promotes differentiation, and contributed to myofibers upon injection into muscle tissue. Using laser scanning confocal microscopy, GFP(+) cells were shown in thin optical sections and 3-D reconstructions to co-express characteristic proteins and to be morphologically indistinguishable from endogenous satellite cells. GFP(+) cells in isolated single myofibers were mononucleate and circumscribed by a membrane, in which α7-integrin, cMet-R, and Myf-5 proteins were expressed. Thus, they were distinct from, yet juxtaposed to myofibers.

However, GFP(+) cells in the satellite cell niche remained constant in number over a 6-month time-period and rarely contributed to the multinucleate muscle fibers with which they were associated. In order to increase their contribution to muscle fibers, a second injury, or metabolic stress, was required. After voluntary exercise on a running wheel, the number of GFP(+) satellite cells per fiber increased less than 2-fold, whereas the number of GFP(+) muscle fibers increased 20-fold over a 6-month period. These results suggest that the GFP(+) cells, which morphologically and biochemically appear to be satellite cells, also appear to function as satellite cells, presumably undergoing sequential asymmetric divisions as they participate in the regeneration of damaged muscle fibers over time. Moreover, these findings provide evidence that the contribution of bone marrow cells to muscle is not a random low frequency event, as it reaches 3.5% under selective pressure of exercise-induced damage over a period of only six months. The increase observed is more than one order of magnitude greater than in any previous reports of bone marrow to muscle (Ferrari et al., 1998; Ferrari et al., 2001; Gussoni et al., 1999). These data demonstrate clearly that a biological step-wise progression from adult bone marrow to muscle-specific stem cell to differentiated muscle fiber occurs in muscle. Such a progression may well be typical of cell type conversions in other tissues.

The lack of evidence that the BMDC could occupy a tissue-localized stem cell niche could have been due to difficulties in identifying tissue-localized stem cells in heart, epithelium, liver, skeletal muscle, and brain, (Bittner et al., 1999; Brazelton et al., 2000; Ferrari et al., 1998; Ferrari et al., 2001; Fukada et al., 2002; Gussoni et al., 1999; Jackson et al., 2001; Krause et al., 2001; Lagasse et al., 2000; Mezey et al., 2000; Orlic et al., 2001). Unlike the satellite cells of muscle, the tissue-localized stem cells in many tissues are often difficult to identify. In the studies reported here applicants capitalized on an advantageous feature specific to muscle: muscle stem cells, or satellite cells, are anatomically and biochemically distinct (Cornelison and Wold, 1997; Mauro, 1961). Moreover, muscle stem cells can be readily analyzed on freshly isolated single muscle fibers, as these preparations include the fiber-associated satellite cells (Bischoff, 1986; Blayeri et al., 1999; Rosenblatt et al., 1995). Although such studies are not easy, due to these properties of muscle, applicants were able to monitor the sequential effects of two distinct damage-inducing procedures and temporally dissociate BMDC conversion to mononucleate satellite cells from their subsequent contribution to multinucleate myofibers. Taken together, these results suggest that BMDC may constitute a previously unrecognized reservoir of cells that is capable of contributing to a tissue-localized stem cell pool, thereby serving as an alternative, or back-up source, of cells for repairing damaged adult tissues.

Bone Marrow Cell Conversion to Muscle Stem Cells is Heritable

If BMDC give rise to muscle-specific stem cells, or satellite cells, they should be heritably altered. Clones derived from single BMDC that are GFP(+) would be expected to express muscle-specific proteins in vivo and in vitro, and be capable of self-renewal and differentiation in tissue culture as well as following injection into muscle tissues of mice. As shown in this report, these characteristics are true of the BMDC satellite cells analyzed here. Depletion of the endogenous satellite cells by irradiation leads to an unoccupied niche. The microenvironment of that niche that normally supports and maintains the endogenous muscle stem cells (satellite cells) can exert similar effects on BMDC that enter that niche. These cells remain quiescent until induced to proliferate, self-renew, or differentiate. Only a subset of the total BMDC satellite cells contribute to myofibers in the absence of further damage, a proportion similar to that observed for clones of primary myoblasts isolated by others (Baroffio et al., 1996; Beauchamp et al., 1999).

Bone Marrow-Derived Satellite Cells Function in Repair of Muscle Damage

Exercise-induced damage to skeletal muscle is associated with satellite cell activation, increased satellite cell number, and an increase in the number of satellite cell-derived nuclei in muscle fibers (Grounds, 1998; Kadi and Thornell, 2000). Based on the observed 20-fold increase in GFP(+) muscle fibers detected in the exercised group of mice, an increase in the number of muscle fiber nuclei that originated from GFP(+) satellite cells is clear. It has long been known that irradiation does not detectably damage mature muscle fibers (Goyer and Yin, 1967; Warren, 1943), but does prevent satellite cells with proliferative potential from participating in regeneration (Gulati, 1987; Rosenblatt et al., 1994). Although the satellite cell population is largely ablated (20% remain) due to irradiation, some remaining satellite cells are presumably the radiation-resistant cells described by others (Heslop et al., 2000) which, together with the proportion of BMDC GFP(+) satellite cells (5%) suffice to allow for muscle regeneration in response to damage such as exercise. The survival advantage of non-irradiated BMDC satellite cells (GFP+) in vivo is further evidenced in culture when the cells are exposed to mitogen rich media that favor proliferation. Similarly, in studies of other tissues, irradiation was necessary for BMDC conversion, for example to liver (Theise et al., 2000; Wang et al., 2002), but usually in conjunction with other strong damage-inducing selective pressures, either genetic or chemical (Ferrari et al., 1998; Gussoni et al., 1999; Lagasse et al., 2000).

Two recent studies suggest, based on karyotype analysis, that the reported instances of bone marrow-derived tissues could be the result of fusion between two cells instead of de novo conversion (Terada et al., 2002; Ying et al., 2002). Fusion does not appear to explain our findings regarding BMDC GFP(+) myoblasts. Within 3.5 days or 5.5 days following isolation, a period during which the cells were primarily attaching to the dishes following tissue dissociation, karyotypes were analyzed. A metaphase chromosome complement of 40, or 2N, was observed in 98% of cells, on a par with wild type controls. These data suggest that instead of fusion, the change in gene expression observed in BMDC satellite cells is due to the microenvironment of the niche they occupy which reveals their inherent plasticity.

Summary

The data presented here suggest that the frequency of conversion of BMDC to a tissue-localized cell type is low unless damage to the tissue occurs. In the absence of damage, the contribution of bone marrow to tissue is a rare, but detectable, event (Castro et al., 2002; Wagers et al., 2002). However, reports that have used damage-induced stress (genetic, chemical, or metabolic) in addition to the irradiation necessary to reconstitute the bone marrow following a transplant provide evidence that damage-induced stimuli increase the conversion of BMDC to tissues other than blood. Thus, applicants propose that the following precepts may be generalizable to studies of BMDC conversions: (1) ablation of the endogenous bone marrow milieu to allow engraftment of donor bone marrow cells, (2) reduction in the number of tissue-localized stem cells to decrease the regenerative potential within a tissue and increase the demand for new cells in that tissue, and (3) exacerbation of the needs of repair and regeneration of a tissue by injury to that tissue in order to increase BMDC contribution.

Further studies are required to define the factors that recruit cells from bone marrow to diverse tissue-localized stem cell niches. Once in the niche, these tissue-localized stem cells can be maintained in a quiescent state. In the case of muscle this period can be at least 6 months, as shown here for GFP(+) satellite cells. In addition, the disparate injury-induced signals resulting from irradiation and exercise that cause the BMDC to become quiescent satellite cells or to proliferate and fuse into multinucleate muscle fibers of the host also remain to be elucidated. Other studies suggest that chemical or genetic damage may release factors key to cell type conversion. Knowledge of the relevant factors may override the need for bone marrow transplantation, which currently serves to mark the cells in order to track them. In addition, identification of the responsible cell type in bone marrow has yet to be resolved and may require retroviral marking or single cell transplantation experiments. An understanding of both the signaling cascades and the origin of the cells responsible will contribute to our overall understanding of stem cell plasticity and its role in development and regeneration.

Experimental Methods

Bone Marrow Transplantation: Marrow was sterilely isolated from 8- to 10-week-old male C57BL/6 transgenic mice that ubiquitously expressed enhanced green fluorescent protein (GFP) (Okabe et al., 1997) and non-GFP, C57BL/6 control mice (Stanford). Donor mice were killed by cervical dislocation, were briefly immersed in 70% ethanol, and had their skin peeled back from a midline, circumferential incision. After the femurs, tibias, and humeri were removed, all muscle was scraped away with a razor blade and the bones were placed in 10 mL of calcium and magnesium-free, Hank's balanced salt solution (HBSS, Irvine Scientific) with 2% fetal bovine serum (FCS) on ice for up to 90 min. The tips of the bones were removed and a 25 gauge needle containing 1 mL of ice-cold HBSS with 2% FCS was inserted into the marrow cavity and used to wash the marrow out into a sterile culture dish. Marrow fragments were dissociated by titurating through the 25-gauge needle and the resulting suspension was filtered through sterile 70 μm nitex mesh (Falcon). The filtrate was cooled on ice, spun for 5 min at 250 g, and the pellet was resuspended in ice-cold HBSS with 2% FCS to 8×10⁶ nucleated cells per ml. Simultaneously, 8- to 10-week-old C57BL/6 mice (Stanford) were lethally irradiated with two doses of 4.8 Gy three hours apart. Each irradiated recipient received 125 μl of the unfractionated marrow cell suspension by tail vein injection within 2 hours of the second irradiation dose.

Muscle Fiber Isolation and Satellite Cell Quantification: Single muscle fibers were isolated from the tibialis anterior according to Rosenblatt et al (Rosenblatt et al., 1995). Briefly, the tibialis anterior was carefully dissected with a razor blade and fine forceps, handling the muscle only by the tendons at the ankle to minimize damage to the fibers. The muscle was then incubated in DMEM/0.2% type I collagenase (Sigma-Aldrich) while constantly rolling at 37° C. for 2 hours. Muscles were triturated using fire polished pipets to gently disaggregate the muscle fibers. Using a dissecting microscope, single fibers were extracted and transferred serially into fresh dishes containing 8 mL of DMEM/10% horse serum (HS) (Gibco)/0.5% chick embryo extract (Gibco) so that no debris surrounded the fibers and their attached satellite cells.

Single fibers were transferred to individual wells of a 24-well plate that were coated with DMEM/10% Matrigel (Beckton-Dickinson). When each well contained one fiber, the plates were placed in a humidified 37° C. incubator for 10 minutes to allow adhesion to the substratum, then 0.5 mL of DMEM/10% HS/0.5% chick embryo extract was added very slowly. Fibers were cultured in a humidified, 37° C., 5% CO₂ chamber for 48-60 hours, and satellite cells crawled off the fiber and attached to the matrix. Typically, using this procedure applicants isolated 12-24 surviving fibers 1-3 mm in length per tibialis anterior. GFP(+) and GFP(−) satellite cells were counted on an inverted stage fluorescent microscope (Zeiss LSM510). Samples were also obtained in DMEM/5% Matrigel coated 4-well chamber slides (Beckton-Dickinson) which were subsequently stained with anti-bodies against c-MetR (Santa Cruz), Myf-5 (Santa Cruz), F4/80 (Caltag), and α7-integrin (Sierra Biosource) to confirm the identity of the migrating satellite cells as described in the following section.

To determine the effects of 9.6 Gy and 18 Gy of γ-irradiation on the endogenous satellite cell niche, mice were anesthetized with IP Nembutal (50 mg/kg) and irradiated inside a lead jig that exposed the right leg and protected the rest of the body. Three weeks post irradiation the animals were sacrificed (n=3 per radiation level) and about 12-20 muscle fibers of similar lengths were isolated from the tibialis anterior of the right and left legs to facilitate satellite cell counting (left leg served as the non-irradiated control).

Immunofluorescence of Isolated Myofibers: Muscle fibers were isolated from bone marrow transplant recipients and littermate controls, as above, and added to poly-L-lysine treated chamber slides (Beckton-Dickinson), that were also coated with DMEM/10% Matrigel, and incubated in a humidified 37° C. incubator for 2 hours to allow adhesion to the substratum. Each well was then carefully filled to maximum capacity (about 2 mL) with 4% EM grade paraformaldehyde (Polysciences) for 5 minutes at 37° C. Samples were blocked for 2 hours at room temperature in PBS/20% normal goat serum (NGS) (Gibco)/0.3% triton-100. Primary antibodies were incubated at 4° C. for 16-40 hours in 0.35% lambda-carraggeennan (Sigma) in the following concentrations: anti-α7 integrin-A954 (1:200, rat IgG clone CA5.5, Sierra Biosource), anti-cMet receptor (1:200, Santa Cruz Biotechnology), anti-Myf5 (1:400, Santa Cruz Biotechnology), anti-GFP (1:1000, Molecular Probes). Secondary anti-bodies (1:400) and nuclear stain, ToPro 3 (1:2000, Molecular Probes), were added in PBS/5% NGS for 2 hours at room temperature. Clone CA5.5 has been shown to specifically stain membranes of primary cultured myoblasts and not NIH3T3 fibroblasts (Blanco-Bose et al., 2001). Three 15-minute washes in PBS were performed between each incubation and after fixation. Cover slips were mounted with Fluoromount-G (Southern Biotechnology Associates) (Beauchamp et al., 2000).

Each fiber was analyzed for antibody staining by laser scanning confocal microscopy (Zeiss LSM510). Data were collected by sequential excitation with different lasers to eliminate any possibility of bleed-through. 1.5 μm optical sections were obtained every 1.0 to 1.5 μm either to visualize individual optical sections or to reconstruct a three dimensional representation of each cell.

Immunohistochemistry: To examine sections of whole TA muscle, bone marrow transplant recipient mice were overdosed with IP Nembutal (150 mg/kg) then perfused with potassium phosphate buffer (0.1 M, pH 7.4) for 3 minutes immediately followed by perfusion with freshly prepared 4% EM grade paraformaldehyde for 15 minutes. Perfusion fixing was necessary in order to retain GFP within cells, as freezing alone led to rapid loss of the GFP signal. The TA was then dissected and frozen in embedding medium (Tissue-Tek, Sakura) and sectioned as 10 μm-thick transverse or longitudinal sections.

Samples were blocked for 2 hours at room temperature in PBS/20% NGS/0.3% triton-100. Primary antibodies were incubated with the sections at room temperature for up to 5 hours in a solution of PBS/5% NGS with antibodies at the following concentration: anti-cMet receptor (1:200), anti-GFP (1:1000). Secondary antibodies and ToPro 3 (1:2000) were incubated with the sections at room temperature for 2 hours in a solution of PBS/5% NGS. Three 15-minute washes in PBS were performed between each incubation and after fixation. Cover slips were mounted with Fluoromount-G. Each section was analyzed as above using sequential laser excitation to eliminate bleed-through.

Exercise Regimen: To determine the effect of exercise-induced damage on donor-derived and endogenous satellite cells, GFP(+) bone marrow transplant recipients were placed in cages with running wheels one week after transplantation. They remained in their “enriched environments” for a period of six months until they were sacrificed for analysis. Littermate controls were maintained as usual in a healthy, but non-stimulating environment.

Myoblast Isolation and Cell Culture: Primary cultures were prepared from muscle slurry according to Rando and Blau (Rando and Blau, 1994). After 9 days of expansion in F-10/20% FBS/bFGF (20 ng/mL) (Promega) cells were released from the collagen coated plate with PBS/0.1 mM EDTA and passed through a 70 μm mesh strainer. After a centrifugation step, cells were stained with an antibody to α7-integrin then double sorted for GFP and α7-integrin expression. Cells were sorted twice using these two markers to reduce the frequency of error to 0.0001 (Moflo, Cytomations). Cells were then replated at clonal density by limiting dilution into DMEM/5% Matrigel (Beckton-Dickinson) rinsed 96-well plates, grown into individual colonies and then switched into DMEM/2% HS for more than seven days to induce differentiation. Differentiated myotubes, pooled populations, or myoblast colonies were fixed with 4% paraformaldehyde for 5 minutes at room temperature, blocked, and stained with anti-GFP (1:1000, Molecular Probes), anti-Desmin (1:400, Chemicon) and Alexa 488 or Alexa 594 (Molecular Probes), respectively, conjugated secondary antibodies (Molecular Probes) and Hoechst 3342 DNA stain (1:1000, Sigma).

Cytology: Cells were harvested from bone marrow transplant crude preparations and GFP(+)/α7-integrin+myoblasts were double sorted 3.5 and 5.5 days post initiation of culture then, side-by-side with control primary C3H myoblasts, were cultured over night in F10/20% FCS/bFGF (20 ng/mL) with 500 μg/mL nocodozole (Sigma). Cells received a hypotonic shock in 75 mM KCl, followed by four rounds of fixation in methanol:acetic acid (3:1), then cells were dropped and dried onto methanol-washed slides where their metaphase chromosomes were stained with Hoechst 3342 and counted. More than 50 spreads were evaluated from each sample.

Myoblast Implantation: SCID mice (Stanford) were anesthetized with IP Nembutal (50 mg/kg), followed by a 10 μL injection of double sorted GFP(+)/α7-integrin+bone marrow derived myoblasts 107 cells/mL in PBS/2% FCS using insulin syringes (Beckton-Dickinson). Seven days following the injection the animals were sacrificed, their TA muscles fixed in 4% pfa, sectioned by cryostat (10 μM), blocked, and stained with anti-GFP (1:1000, Molecular Probes) and anti-laminin (1:200, Chemicon) and Alexa-488 or Alexa-594 secondaries (1:400, Molecular Probes).

### Example 2

### Assays for Key Regulators of Circulating Stem Cells

Here applicants report a robust 5% contribution of adult BMDC to an adult tissue in the absence of ongoing selective pressure and demonstrate that diverse skeletal muscles in mice range 1,000-fold in the frequency with which they incorporate BMDC. The uptake of these cells can be substantial and the differences among muscles are likely to have a physiological basis. BMDC from a transgenic mouse ubiquitously expressing green fluorescent protein (GFP) were tracked as they move into various adult tissues following BMT. Using this approach, applicants show here that in a subcutaneous muscle surrounding the trunk, the paniculus camosus (PC), myofibers expressing GFP are detectable within weeks and compromise one-twentieth of the total muscle fibers by 16 months after BMT. These data demonstrate that this is neither a rare nor subtle event, as large numbers of brightly fluorescent GFP+ fibers are observed per muscle.

The substantial incorporation observed may well be due to the highly regenerative nature of this particular muscle relative to most skeletal muscles, as it is characterized by significantly smaller fiber diameters, increased fiber heterogeneity and an unusually high percentage of centrally nucleated myofibers in the absence of focal injury. By contrast, in muscles such as the tibialis anterior (TA) and diaphragm, which are not highly regenerative in these mice, no central nuclei were observed and only 0.07% and 0.003% of myofibers expressed GFP, respectively. Although the PC has not been extensively studied, it is reported to be a site of exceptionally rapid wound healing and angiogenesis with a plentiful blood supply from overlying dermal vessels. It has been hypothesized that the PC plays a role in maintaining temperature homeostasis, which implies a potentially high contractile activity.

The PC provides a convenient and robust assay for identifying the relevant cell types in bone marrow that contribute to non-hematopoietic tissues as well as key trophic, homing, and differentiation factors responsible for BMDC incorporation in adults. The range in frequencies, which span 3 orders of magnitude among the muscles reported here, suggests that BMDC may incorporate into muscles in a regulated manner according to need and that a comparison may shed light on the molecular basis for the observed differences among muscles.

Bone Marrow-Derived Cells Contribute to Skeletal Myofibers in Diverse Muscles

Following lethal irradiation, recipient mice received intravenous injections of unfractionated bone marrow from an isogeneic, transgenic mouse that ubiquitously expresses enhanced GFP in most cell types including all muscle cell types. When hematopoietic reconstitution was assessed eight weeks post-transplant by FACS, only mice with GFP expression in ≧90% of their circulating nucleated blood cells were analyzed further.

Epifluorescent and laser scanning confocal analysis of 26 skeletal muscles or muscle groups distributed throughout the body resulted in the identification of skeletal myofibers containing GFP in most muscles (Table 3). The morphology of the GFP+ myofibers studied here, like that of their neighbors, demonstrated clear sarcomeric patterns when assessed for both GFP fluorescence and for autofluorescence. GFP+ myofibers were always observed parallel to neighboring myofibers, were of approximately the same diameter as other myofibers, and were frequently greater than 10,000 μm in length.

At 4 and 16 months post-transplant, groups of three mice were euthanized and skeletal muscles throughout each mouse were evaluated for the presence of GFP+myofibers. Although the contribution of BMDC to skeletal myofibers was generally rare (<0.01-0.003%), the detection of GFP+fibers provides evidence that a low level of repair is ongoing even in the absence of overt injury in most muscles. By 16 months post-transplant, several skeletal muscles in the lower leg had modest but reproducibly increased frequencies of BMDC containing myofibers: the TA (0.07%), flexor hallicus longus (0.04%), and the lateral gastronemius (0.04%). However, by far the greatest frequency was observed in the paniculus camosus (PC) in which 5.23% of myofibers expressed GFP. A second muscle with a relatively high frequency was the extensor digitorum longus (0.26%). The incorporation of BMDC for these two muscles was highly significant compared to all other skeletal muscles (P<0.0001 for both the EDL and PC; test for two proportions and Fisher exact test). In addition, the frequency of BMD myofibers was significantly higher in the PC than in the extensor digitorum longus (P=0.0006; test for two proportions). Thus, the PC incorporated BMDC into myofibers with a frequency 20-fold greater than that seen in the EDL, 340 times greater than that seen in the average skeletal muscle, and 1,000-fold more than the frequency seen in several other skeletal muscles (Table 3).

The PC is a thin, subdermal, muscular layer within the superficial fascia that surrounds the entire trunk of animals with a hairy coat. In the mice studied here, the PC has a width ranging from 2-8 myofibers. Superficial to the PC is a well vascularized layer of fat and connective tissue, the paniculus adiposus, on top of which lies the dermis of the skin. Deep to the PC is another layer of fat and connective tissue under which is the potential space that separates when the skin is pulled away from the trunk.

Certain muscle groups were hypothesized to show higher frequencies of BMDC myofiber incorporation due to their higher contractile activity, but did not. Applicants reasoned that this might be the case for the diaphragm, due to its constant workload, but the frequency (0.0028%) was actually reduced relative to other skeletal muscles despite the observation of several long GFP+ myofibers. No BMDC containing myofibers were observed in the extra-ocular muscles, which have drawn interest as they are spared in Duchenne muscular dystrophy and are characterized by an extraordinarily high rate of contraction. Within the tongue, there was a marked tendency for GFP+ myofibers to occur within the centrally located rather than peripheral muscle fibers (0.01% for central myofibers vs. 0.005% GFP+ myofibers in the entire tongue).

GFP+ Myofibers in the PC Appear Morphologically Mature and Express Skeletal Muscle-Specific Proteins

In order to better visualize the length of GFP+ myofibers in the PC, whole skins from five mice that were 10 months post-transplant were mounted between large glass plates and the interior surface of the entire pelt was evaluated. These GFP+ myofibers were frequently as long as other myofibers in their vicinity with an average length of 8000 μm and with occasional fibers exceeding 30,000 μm. The arrowheads indicate two myofiber branch points that, although rare, were consistently found within pelts.

Although the PC surrounds the entire trunk of the mouse, two specific areas within the PC contain the vast majority of GFP+ myofibers in this muscle. A comparison with the diaphragm, a muscle with a low frequency of BMDC incorporation, shows the low abundance of GFP+ myofibers relative to the PC.

The patterns of expression of several skeletal muscle specific proteins, together with the size and sarcomeric patterns confirmed that GFP+ myofibers were typical of differentiated skeletal muscle fibers. Sections of the PC and TA stained with antibodies to the muscle structural proteins myosin heavy chain, desmin, sarcomeric alpha-actin (not shown), and dystrophin (not shown). In addition, each myofiber was ensheathed in laminin, a component of the basal lamina. Staining with five myosin-isoform specific antibodies revealed that GFP+ myofibers, like all myofibers in the PC, homogeneously exhibited the same fast myosin fiber subtype. Specifically, PC myofibers labeled with 2 out of 3 antibodies to fast myosin isoforms (A4.1519+, N3.36+, A4.74−) but not with antibodies against slow (A4.840−) nor fetal myosin (F1.652−) despite clear labeling of appropriate numbers of myofibers in the TA or fetal skeletal muscle with all these antibodies. GFP+ myofibers also exhibited intact neuromuscular junctions when stained with Texas Red-labeled alpha-bungarotoxin, which binds to acetylcholine receptors (ACh-R) at neuromuscular junctions. In all cases, the patterns of antibody staining were indistinguishable from that seen in neighboring non-GFP+ myofibers. GFP+ myofibers lacked expression of the blood lineage marker, CD45, which is expressed by almost all white blood cells, the macrophage marker F4/80, and myeloid cell marker CD11b. Thus, no proteins typical of bone marrow or circulating hematopoietic cells were observed in the GFP+ myofibers, all of which expressed characteristic muscle proteins.

GFP+Myofibers are Formed Continuously Over Time

A time course revealed that GFP+ myofiber formation was not an acute response but increased continuously in an approximately linear manner (R²=0.73). GFP+ myofibers were seen as early as 3 weeks post-BMT, although they were extremely rare at this time point. The linear increase is suggestive of a physiological process in which BMDC continually contribute to myofibers, providing a source of cells to meet the need for myofiber replenishment over time.

The PC is a Highly Regenerative Skeletal Muscle

The main distinction between the PC and the other muscles examined is its regenerative activity. Two morphological features are characteristic of myofiber regeneration in post-natal skeletal muscle: heterogeneous fiber diameters and centrally located nuclei. The incidence of central nucleation is significantly increased in the PC myofibers of both BMT mice and age-matched, non-transplanted mice (P<0.0001 for either compared to TA). Within the PC of BMT mice, 31% of nuclei in GFP+ myofibers are centrally located, 2.5-fold that observed both in GFP-negative myofibers in the PC of the same mice (13%) and in control, PC myofibers in non-irradiated, non-BMT age-matched mice (11%). It is noteworthy that in most skeletal muscles, like the TA, no fibers with centrally located nuclei are observed in the absence of damage such as needle stab injury. In addition, in both non-transplanted and BMT mice the PC exhibits increased myofiber heterogeneity and a smaller mean fiber diameter compared to normal TA (P<0.0001 for both groups). Moreover, The population of GFP+ fiber diameters exhibited increased heterogeneity and a significant shift toward smaller fiber sizes (P<0.001) relative to non-GFP-expressing myofibers. Both the heterogeneous myofiber morphology and increased nuclear centralization suggest that the PC is a skeletal muscle with increased regeneration compared to other skeletal muscles.

Applicants show here that the contribution of BMDC to non-hematopoietic tissues is both measurable and, in some cases, strikingly robust. Indeed, incorporation of BMDC into one tissue, skeletal muscle, can differ 1000-fold. In some muscles, as little as 0.002% (tongue, ribs) of the muscle fibers contained nuclei from GFP+BMDC, whereas in others, such as the EDL and PC, the frequency is as high as 0.26% and 5.2%, respectively. The large range in the frequencies of BMDC contribution to GFP+ fibers suggests that there is a biological basis for this difference. Based on the significantly increased frequency of centrally located nuclei and fiber diameter heterogeneity, applicants speculate that the higher rate of myofiber regeneration in the PC may be responsible for the high frequency of incorporation of BMDC into its myofibers.

In contrast to previous reports, here evidence is provided for a robust contribution of BMDC (>5%) to a non-hematopoietic tissue, the PC muscle, that differs from other skeletal muscles in its rate up uptake of these cells under similar conditions. The differences in BMDC incorporation observed among muscles may well relate to the high regenerative activity of the PC which may reflect increased contractile activity that leads to damage and the need for repair. Indeed, increased exercise is well known to induce increased myofiber heterogeneity and centrally located nuclei in skeletal muscles. Moreover, muscles which normally exhibit a low frequency of BMDC incorporation relative to the PC, can be induced to take up these cells at higher frequencies following an intense six month, exercise-induced, damage regimen. Regardless of the physiological basis for the observed differences, the PC provides an assay system for detecting regulatory factors and for identifying the cell types within the marrow compartment that are responsible for the increased uptake and plasticity of BMDC. A marrow associated regenerative cell may well be capable of serving as a back-up or regenerative reservoir when there is a physiologic or injury-induced need that cannot be met by local tissue-residing stem cells. How or whether such cells are related to other cells defined within bone marrow, such as the hematopoietic stem cells or marrow stromal cells, remains to be determined. A better understanding of the relevant cell type(s) and characterization of the factors that may be involved in recruiting and converting BMDC to non-hematopoietic fates may ultimately lead to novel therapeutic strategies in which the endogenous BMDC of an individual are enlisted to contribute to a wide range of regenerative processes.

Methods

Bone marrow transplantation (BMT): BM was harvested from 8-10 week old, male transgenic mice that ubiquitously expressed an enhanced version of green fluorescent protein (GFP) driven with a B-actin promoter and a CMV enhancer. Briefly, donor mice were euthanized by cervical dislocation, immersed in 70% ethanol, and the skin was peeled back from a midline, circumferential incision. Large limb bones (femur, tibia, & humerus) were surgically isolated and placed in ice-cold of calcium and magnesium-free, Hank's balanced salt solution (HBSS, Irvine Scientific) with 2% FBS for up to 90 minutes. In a tissue culture hood, the tips of the bones were removed and a 25 gauge needle containing 1 mL of ice-cold HBSS with 2% FCS was inserted into the marrow cavity and used to wash the marrow out into a sterile culture dish. Marrow fragments were dissociated by titurating through the 25 gauge needle and the resulting suspension was filtered through sterile 70 μm nitex mesh. The filtrate was cooled on ice, spun for 5 minutes at 250×g, and the pellet was resuspended in ice-cold HBSS with 2% FCS to 4.8×10⁷ nucleated cells per mL.

The marrow of 8-10 week old, isogeneic (C57B/6, Stanford), recipient mice was ablated by lethal irradiation (two doses of 475 cGy, three hours apart). Within the 3 hours following lethal irradiation, each mouse received 6×10⁶ nucleated whole BM cells (in 125 μL HBSS) by tail vein injection. Following the transplant, mice were maintained under standard conditions with a constantly maintained temperature of 20-22° C.

Hematopoietic reconstitution was assayed eight weeks post-transplant by FACS evaluation of the frequency of GFP+ circulating cells. By eight weeks post-transplant, over 95% of recipient mice expressed GFP in greater than 90% of their circulating, nucleated cells. Only mice meeting this criteria were analyzed further.

Muscle tissue preparation: At varying times post-transplant, recipient mice were anesthetized with 60 mg/kg Nembutol and intracardially perfused with 30 mL of 0° C. sodium phosphate buffer (PB, pH 7.4) followed by 30 mL of 0° C. 1.5% freshly dissolved paraformaldehyde (PF) and 0.1% glutaraldehyde. Tissues were harvested, placed in 1.5% PF/0.1% glutaraldehyde/20% sucrose at 4° C. for 2 hours and snap frozen in TISSUE-TEK® O.C.T. compound (Sakura Finetek). 20 to 50 μm thick sections of fixed tissue from over 70 skeletal muscles were cut perpendicular to the orientation of the myofibers on a cryostat.

Muscle survey: Individual muscles were identified and the number and location of each GFP+ myofiber, as well as the total number of myofibers in that muscle, were recorded. Although all GFP+ fibers were counted, in most muscles other than the PC the total number of myofibers present was calculated by counting approximately 1000 fibers, measuring the total area of those fibers, and then extrapolating that number for the total area of that muscle with identical myofiber orientation. All muscles were analyzed in three mice each harvested at 4 and 16 months post-BMT. The frequencies of GFP+ myofibers were compared for statistical significance using the test for two proportions.

PC analysis: Sections of PC were harvested by drawing a grid on the shaved skin of an intact mouse. First, five lines were drawn 0, 1, 2, 3 and 4 cm below the inferior angle of the scapulae and perpendicular to the spine. A vertical centerline was then drawn parallel to the spine. Four additional lines were drawn parallel to the spine 1 or 2 cm either to the left or right of the vertical centerline. The resulting sixteen 1×1 cm squares (4 rows of four squares) of skin were harvested individually. For a comparison of the frequency, GFP+ myofibers among muscles, squares 3b and 3c were counted.

For the time course, groups of five mice were harvested at various time points (2, 3, 5, 12, 16, 23, 50 and 78 weeks) and the PC muscle was evaluated for the presence of GFP+ myofibers. In all cases, the four squares in row 3 were evaluated with the sections cut perpendicular to the orientation of the myofibers. The resulting data were analyzed by standard linear regression.

Immunocytochemistry: Sections were stained with antibodies against muscle proteins including myosin heavy chain (antibody A4.1025; recognizes all myosin heavy chain isoforms; Developmental Studies Hybridoma Bank, Iowa City, Iowa), desmin (Chemicon, Temecula, Calif.), sarcomeric actin (Dako, Glostrup, Denmark), dystrophin (NovaCastra, Newcastle upon Tyne, United Kingdom), neural cell adhesion molecule (Pharmingen, San Diego, Calif.), and basal lamina components such as laminin (Chemicon, Temecula, Calif.) and laminin-β2 (Upstate Biotechnology, Waltham, Mass.). Fiber types in the PC were evaluated by staining with antibodies to specific myosin heavy chain isoforms (all from DSHB, Iowa City, Iowa): A4.840 (Type 1, slow), A4.74 (Type IIa, fast), A4.1519 (Type II, fast), N3.36 (neonatal and Type II, neonatal and adult fast), and F1.652 (fetal). In addition, Texas Red-conjugated alpha-Bungarotoxin (Molecular Probes, Eugene, Oreg.) was used to identify acetylcholine receptors. All sections were blocked with 20% normal goat serum and those using anti-mouse secondary antibodies were blocked with saturating amounts of anti-CD16/32. Muscle sections stained with isotype control primary antibodies and with appropriate secondary antibodies did not display positive staining.

Laser scanning confocal microscopic analysis: Each GFP+ cell was analyzed for antibody staining using epifluorescence (with a long pass filter for GFP that unequivocally distinguishes background from GFP) and 3-dimensional, confocal laser scanning microscopy (Zeiss 510). No GFP+ cells were seen in the muscles of control mice transplanted with unlabeled bone marrow cells. In order to demonstrate colocalization conclusively, confocal parameters were selected to minimize the thickness of the calculated optical section to 1-2 μm despite the lower resolution images produced with these parameters.

### Example 3

### Stem Cell Contribution to Extensor Digitorum Longus Hypertrophy

Experiments in this example demonstrate that bone marrow derived muscle progenitor cells respond to overloading of the extensor digitorum longus (EDL) muscle overloading and mediate hypertrophy and adaptation. This example provides an additional assay system for the contribution of BMDSCs to target tissue.

Surgical removal of the tibialis anterior (TA) muscle is known to result in the selective overloading of the underlying EDL muscle. This results in an adaptive response, which includes extensive hypertrophy from the myofibers of the EDL owing to the abnormally high demand for their use. Hypertrophy and adaptation in the TA ablation model, and in other models of muscle hypertrophy, has been demonstrated to require muscle stem cell activity.

In this experiment, TA muscles were surgically removed from one leg each of 5 GFP-marked bone marrow transplant recipients 4 months after BMT. The surgery was mimicked in the control leg by blunt dissection of the TA from the EDL without resection. Four weeks after the surgery, applicants observed significant hypertrophy of the muscle fibers in the overloaded EDL indicated by an increase in average cross-sectional area of myofibers in overloaded EDL versus control EDL. Typically, applicants have observed little or no GFP+ muscle fibers in the EDL muscles of GFP-BMT recipients, however, following selective overloading of the EDL applicants observed 13-29 GFP+ myofibers/EDL cross-section versus 0-1 GFP+ myofibers/EDL cross-section in the contralateral leg. Moreover, the undamaged, non-overloaded gastrocnemius muscles from both legs showed no significant differences in either average myofiber cross sectional area or in numbers of GFP+ myofibers.

In a second experiment, TA muscles from 3 mice were bilaterally ablated and one leg received an additional 18Gy dose of γ-irradiation following the surgery to prevent the local activity of muscle stem cells. EDL muscles from the non-irradiated legs showed hypertrophy and GFP+myofiber numbers similar to the EDL from the legs with removed TA muscles in the previous experiment. However, the EDL muscles in the irradiated legs showed little sign of hypertrophy or of GFP+ myofibers. These data demonstrate that bone marrow derived muscle precursor cells help mediate adaptation and hypertrophy of selectively overloaded muscle groups.

### Example 4

### Notexin Stimulates BMDC Recruitment

To examine whether the myogenic regenerative capacity of BMDC persists long-term in transplanted animals, applicants evaluated muscle regeneration after acute myofiber injury in mice one-year post-bone marrow transplant. A rapid acute damage model for analyzing focal muscle regeneration was used. This was achieved by injection of the myotoxic snake venom, Notexin (NTX), which results in proliferation and differentiation of muscle satellite cells within days. Notexin was injected into one tibialis anterior (TA) muscle of each mouse while the contralateral TA received PBS. Applicants analyzed five C57/B6 wild type mice (wt mice) that had received a BMT from syngeneic GFP transgenic mice (GFP mice) one year before. All mice analyzed exhibited high level (>90%) multilineage hematopoietic engraftment in their peripheral blood at the time of NTX injection. Transverse sections of TA muscles were analyzed for laminin and GFP transgene expression, and only fibers that were greater than 20 μm in diameter (much larger than blood cells) and with intact basal laminal membranes were scored. One and four weeks after NTX injection, an 8-fold increased number of GFP⁺ fibers was detected compared to the contralateral controls. Thus, the finding that BMDC rapidly contribute to muscle regeneration as long as one year after transplant raised the possibility that these cells persist and are constantly accessible from the circulation.

### Example 5

### BMDC Contribution in a Parabiosis System

As shown above, adult bone marrow-derived cells (BMDC) contribute to myogenesis after bone marrow transplantation (BMT). Here, myogenic BMDC are shown to be present in the circulation using a parabiotic model. Upon transplantation, purified CD45⁺ hematopoietic cells, but not CD45⁻ cells, integrated into damaged muscle fibers. This finding agrees well with data presented in Examples below that a single transplanted hematopoietic stem cell (HSC) yields both blood and muscle. This finding also demonstrates that the observed contribution by BMDCs to muscle are not an artifact created by the treatments involved in bone marrow transplantation. Further, by bypassing BMT applicants have identified more specialized derivatives of HSCs, common myeloid progenitors, which contribute to muscle.

Two questions are addressed here. First, are cells capable of myogenesis always present in the circulation? Second, what is the cellular origin of the BMDC with myogenic capabilities? Possibly the cells derive from muscle tissues, enter the circulation, and gain access to damaged myofibers. In this case, the blood would constitute a route of access to the tissues for already determined precursors. Alternatively, BMDC that participate in myogenesis are derived from hematopoietic cells. In this case, bone marrow cells would undergo a cell fate change and be reprogrammed to express myogenic genes. In this report applicants address these two questions.

To examine whether the myogenic regenerative capacity of BMDC persists long-term in transplanted animals, applicants evaluated muscle regeneration after acute myofiber injury in mice one-year post-BMT. A rapid acute damage model for analyzing focal muscle regeneration was used. This was achieved by injection of the myotoxic snake venom, Notexin (NTX), which results in proliferation and differentiation of muscle satellite cells within days. Notexin was injected into one tibialis anterior (TA) muscle of each mouse while the contralateral TA received PBS. Applicants analyzed five C57/B6 wild type mice (wt mice) that had received a BMT from syngeneic GFP transgenic mice (GFP mice) one year before. All mice analyzed exhibited high level (>90%) multilineage hematopoietic engraftment in their peripheral blood at the time of NTX injection. Transverse sections of TA muscles were analyzed for laminin and GFP transgene expression, and only fibers that were greater than 20 μm in diameter (much larger than blood cells) and with intact basal laminal membranes were scored. One and four weeks after NTX injection, an 8-fold increased number of GFP⁺ fibers was detected compared to the contralateral controls. Thus, the finding that BMDC rapidly contribute to muscle regeneration as long as one year after transplant raised the possibility that these cells persist and are constantly accessible from the circulation.

To address the hypothesis that the relevant cells are constantly present in the blood, applicants analyzed pairs of parabiotic mice surgically conjoined so that they shared the same chimeric circulatory system. Wild type mice were paired with mice transplanted six months earlier with bone marrow from GFP transgenic mice. Three weeks after joining, the peripheral blood chimerism of the wt partners was determined by flow cytometry to be 22-42% GFP⁺. The TA contralateral to the suture site of the wild type partner was then injected with NTX and two or four weeks later, transverse sections of the regenerating TA muscles were analyzed. NTX damaged TA muscles of the wt partners contained GFP⁺ myofibers with centrally located nuclei typical of regenerating muscle and intact basal laminal membranes. Mechanical damage related to the parabiosis surgery also led to the contribution of BMDC to a few myofibers in the ipsilateral leg at the suture site. These results demonstrated that BMDC can contribute to muscle fibers in the absence of toxin induced damage, but that the incidence may be lower. This frequency is markedly increased and the time course shortened by experimental induced damage. Taken together, these data demonstrate that BMDC with a capacity to participate in myogenesis persist, circulate and are available for recruitment during muscle regeneration in mice that were never exposed to local or total body irradiation or forced marrow mobilization.

Parabiosis experiments were then designed to test whether variables associated with BMT and irradiation of the donor were critical to the contribution of BMDC at the site of injury in the focally damaged partner. To this end, pairs of parabiotic GFP transgenic mice, which had not been transplanted with bone marrow, and wt mice were analyzed. As in the previous parabiotic pairs, TA muscles of the ipsilateral leg showed little evidence of regeneration. However, cells from the GFP transgenic mouse clearly contributed to the NTX-induced muscle regeneration in the contralateral TA of wt partners. Furthermore, because the number of fibers observed per section was similar for both types of parabiotic pairings, applicants concluded that the active population of bone marrow-derived cells with myogenic potential can be functionally reconstituted by bone marrow transplantation. Although in the wt:GFP parabiotic pairs applicants cannot be certain that the cells responsible for the muscle regeneration are of bone marrow origin, the experiments with bone marrow transplanted parabiotic pairs provide evidence that this was the case. These results with parabiotic mice with and without BMT indicate that the variables associated with the transplant procedure, such as irradiation that results in vascular damage or a “cytokine storm”, or marrow harvest-induced stresses that result in atypical mobilization, are not required for the observed contribution of BMDC to muscle fibers.

To identify the cellular origin of the BMDC with myogenic potential, applicants first fractionated total bone marrow into hematopoietic and non-hematopoietic cells based on their expression of the pan-hematopoietic marker CD45. The two fractions of transplanted GFP-expressing bone marrow cells (CD45⁺ and CD45⁻) were sorted from GFP transgenic Ly5.2 mice. Each fraction was mixed with the appropriate unlabeled complementary bone marrow fraction from non-GFP C57/B6 Ly5.1 mice. Five months post transplantation (at the time of NTX injection), mice from three separate experiments (n=18) exhibited either GFP⁺ Ly5.2⁺ or GFP⁻Ly5.1⁺ blood chimerism in accordance with the genotype of the original CD45⁺ fraction. One month after NTX injection, GFP⁺ myofibers were detected only when GFP⁺ CD⁴⁵⁺ bone marrow cells had been transplanted. Thus, although the CD⁴⁵⁻GFP⁺ cells were more abundant in these experiments than in normal unfractionated total bone marrow transplantation experiments (3-5 fold higher), they were not able to participate in muscle regeneration. This finding suggests that under the experimental conditions used here, non-hematopoietic stem cells that might be present in the marrow do not participate in muscle repair following BMT.

Applicants and others have recently demonstrated that the myogenic contribution from BMDC is of hematopoietic origin. Briefly, single GFP⁺ HSC (Lin⁻ c-kit⁺Sca-1⁺) that were also contained in the verapamil-sensitive side population were double-sorted. Individual GFP⁺ HSCs were then transplanted into lethally-irradiated Ly5.2 recipients together with GFP⁻ bone marrow cells depleted of HSCs with long term reconstitutive activity (Sca-1⁻ fraction). Analysis of the nucleated blood cells from these mice 2-6 months after transplantation, revealed circulating donor derived GFP⁺ hematopoietic cells in the peripheral blood of the recipients and numerous GFP⁺ myofibers in the muscles of 8 mice which had reconstituted their blood from a single HSC. The finding that both GFP⁺ hematopoietic cells and GFP+ myofibers could be detected in the same animal transplanted with a single GFP⁺ HSC unequivocally demonstrates that this cell can give rise to both blood and muscle.

To determine which subset of bone marrow cells contributed to muscle, marrow was further fractionated using well-documented markers. Fractions were injected directly (IV or IM) into regenerating muscle in order to bypass the need for bone marrow reconstitution and allowing us to test more specialized derivatives of HSC. As a control, total GFP⁺ bone marrow was injected IM into TAs of 3 wt mice that were treated with NTX to stimulate muscle regeneration. Three weeks later the TA muscles of 2 of 3 mice showed evidence of GFP⁺ muscle fibers with centrally located nuclei, as expected from previous studies with whole bone marrow (2).

Experiments were designed to determine whether mature macrophages participate in myogenesis. These cells have been considered by others to be the prime candidate for an HSC derivative with this function due to their innate fusogenic activity. Three lines of evidence are presented that argue that macrophages within bone marrow are not involved in this process. First, in some experiments using IV and IM injections of whole bone marrow, GFP⁺ cells were observed in clusters of the size of muscle fibers yet without a surrounding basal laminal membrane and no GFP⁺ muscle fibers were found. These GFP⁺ clusters stained strongly with a mature monocyte/macrophage marker CD11b (Mac1) and were consistent with the appearance of phagocytic macrophage cells engulfing dying myofibers and participating in a process of muscle fiber degeneration, not regeneration. True GFP⁺ myofibers found in control BMT mice did not express CD11b. Because of the abundance of macrophages engulfed in damaged fibers and the total absence of GFP⁺ myofibers, these observations provide the first line of evidence that the fusion of macrophages with damaged fibers is not the mechanism by which GFP⁺ myofibers arise. In a second type of experiment, no fusion was observed between macrophages and myogenic cells in tissue culture under conditions in which each cell type fused to itself. Third, when CD11b⁺ cells were injected IM into regenerating TA muscles of 7 mice, no GFP⁺ myofibers were detected after 3 weeks. By contrast, as mentioned above, in control mice that had received an IM injection of whole bone marrow, several laminin ensheathed myofibers were observed. Taken together, these results suggest that cells exist within whole bone marrow that can regenerate muscle, but they do not include CD11b⁺ mature monocytes/macrophages isolated from the bone marrow.

Whole bone marrow was further fractionated by FACS to enrich for HSCs and more specialized HSC derivatives or progeny. Expression of the lineage markers (Lin: Ter119, B220, CD3, Gr1 and CD11b), the c-kit tyrosine kinase receptor and the cell surface antigen Sca-1 were tested. Fractions containing c-kit⁺ Sca-1⁻ cells and c-kit⁺ Sca-1⁺ cells were injected directly into regenerating TA muscles of wt mice and found to contribute to muscle fibers, whereas c-kit⁻ Sca-1⁻ and c-kit⁻ Sca-1⁺ cells did not. Similar data were obtained following IM injection of c-kit⁺ Lin⁻ and c-kit⁺ Lin⁺ fractions. Only c-kit⁺ Lin⁻ cells demonstrated contribution to muscle. However, the ability of BM cells to incorporate into muscle did not specifically co-segregate with the expression of Sca-1. Together, these data suggest that the cells in the bone marrow that integrate into myofibers during muscle regeneration are from the Lin⁻ c-kit⁺ fraction of bone marrow cells containing both stem cells (Sca-1⁺) and HSC derivatives, i.e. hematopoietic progenitor subsets (Sca-1⁻). That GFP⁺ myofibers were detected following injection of the c-kit⁺ Sca-1⁺ fraction is in good agreement with findings showing that individual transplanted HSCs can contribute to muscle regeneration. However, these data go further in that they identify a common myeloid progenitor (Lin⁻ c-kit⁺ Sca-1⁻) downstream of HSCs, a more specialized HSC derivative that no longer has the capacity for long-term hematopoietic reconstitution but is still capable of myogenesis.

In summary, this study demonstrates for the first time that cells capable of rapidly repairing muscle persist for as long as a year post-transplant. In addition, it was previously assumed that this potential was linked in some manner with transplantation-associated factors such as the reconstitution of blood lineages, forced mobilization of bone marrow cells, and lethal irradiation resulting in cytokine dysregulation. The parabiosis experiments presented here show that this is not the case. Moreover, they demonstrate that BMDC cells can circulate and can integrate into damaged skeletal muscle throughout life. This finding is in contrast with previous reports in which no evidence of BMDC contribution to muscle regeneration was seen with single cell HSC transplants or in parabiotic mice, presumably because no damage was induced. Another unresolved question was the nature of the cell within bone marrow capable of contributing to muscle. Applicants and others have shown recently that these cells are of hematopoietic origin. First myogenic and hematopoietic activity were found to co-segregate in the CD45⁺ bone marrow subpopulation and second, a single hematopoietic stem cell was shown to contribute to both blood and muscle when transplanted into a lethally irradiated animal. To identify the relevant HSC derivatives, applicants used bone marrow fractionation and IM injections to overcome the need for engraftment and reconstitution of the hematopoietic cells ablated by irradiation. This approach allowed us to identify HSC-derived hematopoietic fractions that were no longer capable of reconstituting all lineages of the blood, but were able to contribute to muscle regeneration. As a result, applicants showed that not only HSCs but also HSC derivatives, common myeloid progenitors (Lin⁻ c-kit⁺ Sca-1⁻), can participate in muscle regeneration.

A major controversy in the field, that there is little evidence for HSC contribution to myogenesis, has been resolved here. Damaged muscle is a requirement for the high levels of BMDC incorporation into myofibers. The findings in both papers are in agreement in that (1) BMDC can contribute to muscle in several experimental paradigms and (2) a single HSC can both reconstitute the hematopoietic system and give rise to muscle. Applicants have further extended these findings by showing that a more specialized progenitor, an HSC derivative, which has lost the ability to reconstitute the hematopoietic system, retains the capacity to contribute to damaged muscle.

The data presented here suggest the following model for the contribution of circulating bone marrow derivatives to muscle. HSCs (CD45⁺ Lin⁻c-kit⁺ Sca-1⁺) capable of reconstituting cells of the blood lineages following BMT into lethally irradiated animals are capable of participating in myogenesis. Furthermore, more specialized CD45⁺ Lin⁻ c-kit⁺ Sca-1⁻ progeny, derivatives of HSCs (myelomonocytic progenitors) present in bone marrow that have lost the capacity to reconstitute the blood, are also capable of contributing to damaged muscle fibers. By contrast, their more mature derivatives within bone marrow, macrophages, no longer have the capacity to contribute to myogenesis. Applicants therefore speculate that cells early in the myeloid lineage are altered by the microenvironment of damaged muscle. These myeloid progenitor cells circulate within the vasculature and are available for recruitment to muscle damage throughout the life of the animal in the absence of BMT related perturbations such as mobilization of cells or irradiation induced damage, as shown in our experiments with parabiotic pairs of mice. In the case of mesoangioblasts, a therapeutic effect has recently been shown for one type of muscular dystrophy. If other cellular sources, such as the hematopoietic cell progeny identified here, can function similarly, they would clearly be advantageous as they are more readily accessible.

### Example 6

### Bone Marrow Derived Purkinje Cells

These experiments demonstrate that bone marrow-derived cells cross the blood-brain barrier and contribute to neurons, particularly Purkinje cells, in the CNS of human patients. Purkinje neurons are generated only during early brain development. In humans, generation of Purkinje neurons starts at 16 weeks of gestation and is complete by the end of the 23rd week. Most of the maturation of the characteristic dendritic trees of human Purkinje neurons is finalized during the first year of life. By contrast to other neurons in the adult brain, there is no evidence for the generation of new Purkinje neurons after birth, even in cases of severe Purkinje cell loss caused by trauma or genetic disease.

The human brain contains 15 million Purkinje cells, which are among the largest neurons in the CNS. A typical Purkinje neuron has >50-fold the volume of neighboring neurons in the brain, and its complex dendritic extensions receive inputs from as many as one million granule cells. Purkinje cells play vital roles in maintaining balance and regulating movement. A loss of Purkinje cells results in deficits in these functions in several disorders: ataxia-telangiectasia, the most common cause of progressive ataxia in infancy; Menkes' Kinky Hair syndrome; the alcoholic cerebellar degenerations, particularly Wemicke-Korsakoff syndrome; and various prion diseases including scrapie, Creutzfeldt-Jakob, and Kuru. Thus, renewal or rescue of Purkinje neurons has significant therapeutic implications.

In control male and female cerebellar sections processed for in situ hybridization, human X and Y chromosomes can be readily visualized with the specific, labeled probes. Large, yellow, pear-shaped Purkinje neurons are easily recognized between the cell-sparse molecular layer containing stellate and basket neurons and the inner granular layer, composed primarily of small granule neurons and a few Golgi neurons. The characteristically large size of the Purkinje cells and thick dendritic projections that extend into the molecular layer are readily apparent. Nuclei of Purkinje cells, visualized as blue when stained with To-Pro-3, have typical diffuse chromatin and a distinctive large nucleolus, whereas the nuclei of the neurons in the surrounding granular layer have very little cytoplasm, small nuclei with densely packed chromatin and no obvious nucleolus. Thus, these cell types are easily distinguished by histology after in situ hybridization without the need of antibody staining, an assay precluded by the digestion procedure.

In situ hybridization revealed that X and Y probes yielded red and green signals that clearly distinguished the two sex chromosomes by confocal microscopy. The Vysis X chromosome probe is conjugated to Spectrum orange that fluoresces at a peak of 588 nm (red), whereas the Y chromosome probe is conjugated to Spectrum green that fluoresces at 524 nm (green). Fortuitously, the autofluorescence in the green and red channels superimposed to yield a yellow color that allowed distinction of the Purkinje cell body cytoplasm. In cerebellar sections from control female brains, Y chromosome labeling was never detected. Two female Purkinje cells and three male Purkinje cells were visualized between the cell-sparse molecular layer and the granular layer. Note that two sex chromosomes were not always seen in every control Purkinje nucleus because of the thin sections required. In contrast, the nuclei of most of the smaller granule neurons exhibit staining of two chromosomes, as the entire nucleus is usually contained in the section. However, in 10-μm sections two or more granule neuron nuclei may be superimposed, giving the impression of more than two sex chromosomes per cell. It was possible to verify that each granule neuron nucleus contained only two sex chromosomes by examining individual serial optical sections within the stack. Occasionally, the X chromosome or the Y chromosome appears to be outside or proximal to the Purkinje nucleus, but this is caused by the projection of stacked serial confocal images. This finding was confirmed by examining individual 1-μm optical sections within the stack that are sufficiently thin to permit precise cellular localization of the chromosome (not shown). On the other hand, in some cases, a chromosome belongs to an abutting cell, which is evident from the cytoplasm separating the two cells. In the granular cell layer many cells can be seen with one X and one Y chromosome. Because these cells are small and densely packed with little cytoplasm, it is often difficult to distinguish the borders between adjacent cells, a problem not encountered with Purkinje neurons because of their large size and abundant cytoplasm.

Cerebellar tissue samples obtained at autopsy were analyzed from female patients with hematologic malignancies. Initially, chemotherapy was accompanied in most patients by total body irradiation to reduce the malignant cell population and decrease rejection of donor cells. In a few cases marrow cells from male donors were then infused into female patients, whereas most received sex-matched bone marrow. Immunosuppressive agents were given to decrease graft-versus-host reactivity. The four subjects of the study were selected based on the following criteria: sex (male donor and female recipient), availability of brain tissue, survival for 3-15 months posttransplant, and death unrelated to CNS complications. Five female patients transplanted from female donors were chosen as controls by using the same criteria. Cerebellar tissue sections were cut and coded to ensure patient anonymity and “blinded” analysis.

Examination of cells within blood vessels and parenchyma of cerebella underscored the high degree of specificity of the Y chromosome probe. In sections from all of the sex-mismatched transplant patients, Y and X chromosomes were found in numerous cells, presumably blood cells, within the lumina of cerebellar vessels. Variation among patients may have resulted from differing degrees of hematopoietic reconstitution that was not determined years ago when these patients died and could no longer be ascertained. In sex-mismatched transplant patients, an occasional male donor-derived cell (Y chromosome in nucleus) was found in the granular cell layer, whereas a Y chromosome was never found in the granular cell layer of female patients who received a bone marrow transplant from a female donor. Cells in the parenchyma are likely to be macrophages and microglia that are well known to be derived from bone marrow. Because of the inability to perform immunohistochemistry on these highly digested tissues, the specific identity of these cells could not be discerned, because unlike Purkinje cells, their morphology was not distinct.

Male chromosomes were readily detected by epifluorescence in the relatively thin sections of Purkinje neurons from female brains. Following along the border of the dendritic layer, each Purkinje cell was examined for the presence of a green-labeled Y chromosome, and those with Y chromosomes were then imaged at high resolution with the confocal scanning laser microscope. Y chromosomes were found in four of the total 5,860 Purkinje nuclei examined by epifluorescence in sex-mismatched transplant patients. No Y chromosomes were found in Purkinje nuclei from sex-matched transplant patients (controls). In rare cases, the X chromosome assumed a dumbbell configuration. The distance between the two red spots in 14 different X chromosomes that had dumbbell shapes averaged 1.1±0.3 μm and the greatest distance between two such spots was 1.9 μm. Dumbbells were not caused by radiation and bone marrow transplantation as they are routinely observed in normal cells, as discussed in the Vysis protocol booklet, in which criteria are provided to distinguish a single chromosome with a dumbbell shape from two distinct chromosomes. Analysis of distances between the two red spots allowed distinction of whether such signals derived from one or two chromosomes (see below).

In two of the Purkinje cells analyzed, three sex chromosomes were observed within the same Purkinje nucleus. In one case, a Y chromosome was detected together with two X chromosomes in a serial stack of optical confocal images. In another case, one of the randomly scanned Purkinje cells was found to contain three X chromosomes. No dumbbells were evident. Indeed, the closest chromosomes in the cells with three chromosomes were >4.0 μm apart. Thus, it is highly unlikely that the probe bound parts of a single chromosome. Notably, the finding of these two cells with more than a diploid sex chromosome composition raised the possibility that the contribution of donor-derived bone marrow cells to the Purkinje neuron population might occur by fusion of these two cell types.

Although the possibility remains that the Purkinje cells with one X and one Y chromosome arose de novo from cells within the bone marrow, an argument based on sampling can be made in support of cell fusion. Each of the cells contain only two sex chromosomes, which might suggest that they resulted directly from a male stem cell present in the bone marrow that changed to become a Purkinje neuron in the brain. On the other hand, because less than half of a Purkinje cell nucleus was encompassed in the sections analyzed, it is quite possible that our analyses did not include all sex chromosomes present in a given Purkinje cell. Nonetheless, whenever a Y chromosome was detected, an X chromosome was also present. To address the possibility that the sex chromosomes were underrepresented in our sample, the probability of observing zero, one, or two chromosomes in a section containing less than half of a Purkinje cell nucleus was determined. A total of 214 randomly scanned cells were selected and analyzed for the number of sex chromosomes they contained by reconstructing a series of optical sections obtained from confocal images. The results revealed the following frequencies of X chromosomes in optical sections: 32% contained zero, 46% contained one, and 21% contained two chromosomes. Thus, in diploid cells only one-fifth of randomly sampled 10-μm sections of Purkinje nuclei exhibited the full complement of sex chromosomes. As a result, the finding of an X and a Y chromosome in the same partial Purkinje cell nucleus may well underestimate the total number of sex chromosomes in that cell. In addition, the low frequency of a diploid chromosome content suggests that detection of three chromosomes would occur in less than one-fifth of all cells analyzed and that the probability of detecting four chromosomes would be exceedingly low. Taken together, this analysis and the data indicate that cell fusion occurred.

The data presented show that adult human bone marrow cells can contribute to mature Purkinje neurons in adult women with hematologic malignancies. Even though this is not a frequent event (0.1% of the cells examined), it is surprising that it occurs at all because the generation or repair of these cells after birth had not been documented. Because there are 15 million Purkinje cells in the human adult brain, by extrapolation, the total number of cells affected by a bone marrow transplant could be quite substantial.

Methods

Tissue Specimens. At death, brains were removed and fixed intact in neutral buffered formalin (3.7-4.0% formaldehyde) for 10-14 days. Tissue blocks were then embedded in paraffin. For this study, 10-μm sections were cut from cerebellar tissue of each transplant patient and from untransplanted male and female control brains and mounted onto glass slides. Only half of a Purkinje cell nucleus could be included in these 10-μm sections; thicker sections could not be used because Y chromosomes could not be identified by epifluorescence before in-depth confocal analysis.

In Situ Hybridization. Paraffin was removed from sections with three changes of xylene (10 min each), rehydrated through graded alcohols (3 min each), and washed twice with double distilled water (ddH2O). Sections were placed in 0.2 M HCl at room temperature for 15 min and rinsed twice in ddH2O and once in Tris-EDTA. Sections were then digested in Proteinase K (3.8 μg/ml Tris-EDTA) at 37° C. for 37 min and rinsed twice with ddH2O and once with 2×SSC. Slides were placed in preheated pretreatment solution (sodium isothiocyanate, Vysis, Downers Grove, Ill.) at 82° C. for 37 min followed by three rinses at room temperature with 2×SSC. Sections were digested in protease I (pepsin) 4 mg/ml in protease I buffer (Vysis) for between 10 and 37 min (the time differing depending on fixation of sample), followed by three rinses in 2×SSC. Sections were denatured for 5 min at 73° C. in 49 ml of formamide (fresh or frozen aliquots)/7 ml of 20×SSC/14 ml of ddH2O, then dehydrated through a graded series of ethanols. A CEP XY DNA probe (Vysis) was applied to each section, sealed under a glass coverslip, and incubated overnight at 42° C. The Vysis probes detect the alpha satellite sequences in the centromere region of the X chromosome (DXZ1 locus) and the satellite III heterochromatin DNA at the Yq12 region of the Y chromosome (DYZ1 locus) (see Vysis). The next day, coverslips were removed in 2×SSC, rinsed for 2 min in 2×SSC/0.1% Nonidet P-40 at 73° C., allowed to air dry, and mounted in DAPI II mountant (Vysis) to which To-Pro-3 iodide (Molecular Probes) was added at a dilution of 1:3,000.

Microscopy. Cerebellar sections were viewed at ×63 by using a Zeiss LSM510 laser scanning confocal microscope equipped with epifluorescence. The margin between the granular cell layer and the acellular molecular layer was scanned for Purkinje cell bodies and the presence of a Y chromosome by using epifluorescence. The green Y chromosomes were evident as a lime-green dot in the midst of the yellow-green autofluorescent cytoplasm. A total of 5,860 Purkinje cells from sex-mismatched bone marrow transplants and 3,202 Purkinje cells from sex-matched transplants were counted and assessed for the presence of a Y chromosome (green, visible by epifluorescence), allowing a determination of the overall frequency of Y chromosome-containing cells. As part of the blind study, images of every 20th Purkinje neuron were scanned on the confocal microscope by acquiring 1-μm serial optical sections through the portion of the nucleus present in the section (<50%). The stacks of images were then used for further analysis of the sex chromosome content of Purkinje cells in general and the frequency of zero, one, and two sex-chromosomes within nuclear sections of the size analyzed here. From all of the control and test Purkinje cells serially scanned and reconstructed, a total of 214 nuclei were used to assess the average number of sex chromosomes in randomly sampled Purkinje neurons.

### Example 7

### Bone Marrow Derived Cells Fuse with Purkinje Cells

As described herein, BMDCs can contribute to the regeneration of neural tissue. Experiments described in this example demonstrate that, in the case of Purkinje cells, the contribution of BMDCs to neural tissue occurs by fusion of BMDCs with neurons to produce stable heterokaryons. The previously unrecognized finding that binucleate, chromosomally balanced heterokaryons are produced in vivo in tissues such as brain is remarkable, as stable heterokaryons were only thought to occur artificially in tissue culture. In these in vivo heterokaryons, the neurons were dominant over the BMDCs, as no mitosis was evident and the morphology was typical of functional Purkinje neurons, with complex dendritic trees and axons. Moreover, cytoplasmic factors within the Purkinje neurons reprogrammed the fused BMDC nuclei resulting in nuclear swelling, decondensed chromatin and activation of a Purkinje neuron-specific transgene, L7-GFP.

Purkinje neurons are mononucleate diploid cells that are generated only during gestation and not replaced after loss through trauma or genetic disease. The complexity and importance of the Purkinje neuron is underscored by the fact that the axons of the Purkinje neurons are the only efferent from the cerebellum to other brain regions, and in humans each Purkinje neuron can receive over 1 million inputs from other neurons. Indeed, these large, highly specialized, Purkinje neurons of the cerebellum are critical to balance and fine motor control, and defects in these cells result in ataxias.

To elucidate the mechanism by which BMDCs contribute to neural tissue, the bone marrow from transgenic mice ubiquitously expressing GFP was harvested and transplanted by tail-vein injection into lethally irradiated syngeneic recipient mice. Several months later, Purkinje neurons expressing GFP were detected in the cerebella of recipient animals. These GFP-positive Purkinje cells were indistinguishable from normal Purkinje neurons, with their soma in the Purkinje cell layer (PCL) and a large, apical and highly branched dendritic tree that extended into the cell-sparse molecular layer. The single axon from the Purkinje neuron extended through the granular cell layer (GCL) into the white matter and was the only output axon from this neuron to other brain regions. An image of a bone-marrow-derived Purkinje neuron at low magnification shows this cell in the context of a cerebellar lobe. At higher magnification, laser-scanning confocal microscopy reveals part of the descending axon and many small synaptic spines on the extensive dendritic tree. Other GFP-positive BMDCs, such as microglia and macrophages, were readily apparent throughout the brain. The architecture and structure of the dendritic tree of the GFP-positive Purkinje cell with its many synaptic spines are indistinguishable from typical Purkinje neurons and are characteristic of healthy functioning neurons.

Applicants investigated whether GFP-positive Purkinje neurons expressed genes that are typically found in Purkinje cells, bone marrow cells, or both. When analysed by immunofluorescence microscopy, all of the GFP-positive Purkinje neurons strongly expressed the calcium-binding protein, calbindin, a hallmark of the Purkinje cell type. No other cell type expressed calbindin in the cerebellum. To assess whether they still expressed markers typical of bone marrow cells, GFP-positive Purkinje neurons were assayed for haematopoietic markers. Sections containing BMDC Purkinje neurons were stained with antibodies against CD45 (a pan-haematopoietic marker), CD11b (a macrophage/microglia marker), F4/80 and Iba1 (microglial markers). The GFP-positive Purkinje neurons were negative for all four of these haematopoietic markers, suggesting that the genes encoding these products were either inactivated or never expressed in the BMDCs that resulted in the GFP-positive Purkinje neurons in the brain. The BMDCs also yield other cell types in the cerebellar parenchyma, including GFP-positive microglia and macrophage cells. As expected, these GFP-positive BMDCs expressed haematopoietic markers. Thus, co-expression of Purkinje neuron gene markers and haematopoietic markers was not observed.

Applicants then determined the time course of BMDC contribution to the Purkinje cell pool. Mice were transplanted with bone marrow at two months of age and the number of GFP-positive Purkinje neurons detected in 20 mice under nonselective conditions was scored over a period spanning 1.5 years, approximately 75% of the average mouse lifespan. GFP-positive neurons were not apparent until several months after transplantation, and the maximum number observed under these nonselective conditions was 60 neurons in one animal after 1.5 years. A linear increase in GFP-positive neurons was observed that correlated with age, a pattern that was statistically significant up to 16 months after transplantation.

Applicants analysed the nuclear composition of the GFP-positive Purkinje neurons to determine whether they arose de novo from BMDCs or through fusion to endogenous Purkinje neurons. Using a laser-scanning confocal microscope, serial 1 μm optical sections were obtained through the entire cell body of GFP-positive Purkinje cells. Serial reconstruction of these cells revealed that in the more than 300 cases where it was possible to image the full extent of the soma, two nuclei were always detected. A typical GFP-positive Purkinje neuron with an axon exiting the soma from the top right and a primary dendrite with several secondary and tertiary dendrites is shown. As with all of the GFP-positive Purkinje neurons, numerous small synaptic spines (the post-synaptic specializations of active synapses) were readily apparent on the dendrites. The endogenous Purkinje nucleus of the recipient was enlarged, with dispersed chromatin and a prominent nucleolus, similar to other neighboring Purkinje nuclei. By contrast, the putative bone-marrow-derived nucleus that fused into the host Purkinje neuron contained compact highly condensed chromatin. These results indicate that BMDCs contribute to the Purkinje neurons by fusion and not by de novo neurogenesis.

To determine definitively the cellular origin of each nucleus within GFP-positive Purkinje neurons, bone marrow from male donor mice was transplanted into female recipient mice using the same experimental paradigm described above. The presence of a male nucleus was assayed using a Texas-Red-labeled DNA probe specific to the Y chromosome and examination by fluorescence in situ hybridization (FISH). One year after transplantation, brains were sectioned at 12 μm and serial cerebellar sections were stained for GFP and counterstained with the nuclear stain To-Pro3 to visualize nuclear DNA. Serial 1 μm optical sections were obtained to determine the number of nuclei in GFP-positive cells. A motorized stage was used to record the x, y and z coordinates, ensuring the precise relocation of GFP-positive cells after the proteinase K digestion and FISH staining protocol, which removes most of the GFP staining. Representative examples of GFP-positive Purkinje neurons with two distinct To-Pro3-labelled nuclei were visualized. After FISH, a red Y-chromosome was detected in one of the two nuclei in each cell. The two sets of panels in this figure show cells in the same locations before and after the extensive proteinase K digestion of the tissue that is necessary for FISH. The other nucleus within the GFP-positive soma is the endogenous cell nucleus of the Purkinje neuron that does not contain a Y-chromosome. GFP-positive soma was found in two adjacent sections. The chromatin in the donor-derived Y-chromosome-positive nucleus of this cell was as dispersed as the host nucleus, with a prominent nucleolus; a structure not seen in the compact chromatin characteristic of marrow-derived cells. Donor-derived microglial cells were evident in the host tissue, and these cells also contained a Y chromosome. Despite the change in nuclear morphology, there was no evidence of cytokinesis or karyokinesis in any of the cells analysed. Indeed, the fused cells seemed to be stable heterokaryons that persisted over time. Furthermore, there was no evidence of GFP-positive Purkinje neuron death, such as blebbing or membrane fragmentation, among the more than 300 GFP-positive neurons examined. These data indicate that the GFP-positive Purkinje neurons found in the host cerebellum are the result of fusion between a host female Purkinje cell and a male BMDC.

During the course of this analysis, Applicants observed that the structure of the two nuclei differed markedly among the GFP-positive Purkinje cells. Approximately 50% of the GFP-positive cells contained one large ‘Purkinje-like’ nucleus, with dispersed chromatin, and one small ‘bone-marrow-like’ nucleus, with compact chromatin. In the other cells scored, both nuclei appeared Purkinje-like. A time course of chromatin alteration demonstrates that the ratio of nuclei with dispersed-to-compact chromatin in the cerebella of individual mice increased over time. These data suggest that once a BMDC with a compact nucleus fuses to a Purkinje neuron, the bone-marrow-derived nucleus becomes less compact and dense and finally assumes the morphology of the Purkinje nucleus to which it fused. This increasing trend towards dispersed chromatin in the fused BMDC nucleus over time suggests that the fusion events are stable.

The activation of previously silent genes by intracellular signals generated by one of the two nuclei in a heterokaryon has been well established in vitro. To determine whether the changes in chromatin structure observed in the fused BMDC nuclei correlate with reprogramming and activation of Purkinje genes, a transgenic mouse that expresses GFP under the control of the Purkinje-specific promoter, L7-pcp-2 was used as a bone-marrow donor. The previously described expression pattern of the L7-GFP promoter was confirmed by analysing sections from the brain of these transgenic mice. In the brain, the only GFP-positive cells detected were the Purkinje neurons. Flow cytometry analysis of the L7-GFP bone marrow showed that these transgenic mice do not express GFP in their bone marrow. Indeed the fluorescence-activated cell sorting (FACS) plots of the L7-GFP transgenic cells were indistinguishable from those of wild-type marrow and were three orders of magnitude lower than the GFP fluorescence obtained from GFP-positive bone marrow. Thus, the L7-GFP transgenic promoter is inactive in the bone marrow of this mouse line.

Four mice were sacrificed five months after receiving a bone marrow transplant from the L7-GFP mouse and then analysed. L7-GFP-positive Purkinje neurons were found in the cerebella of all four mice, and on average 2-3 fully mature GFP-positive neurons were observed in each mouse, correlating with the prediction for five months after transplantation. All of the L7-GFP-positive Purkinje cells contained two nuclei. In certain cells, one nucleus was evident in the confocal image, whereas the other was in a different plane of focus. Donor-derived haematopoietic cells such as microglia and macrophage cells are known to be present in the brain parenchyma after a bone marrow transplant, but these donor-derived cells did not express GFP, a further indication for the specificity of the L7-pcp-2 promoter. These results demonstrate that under physiological conditions, transplanted BMDCs not only fuse to pre-existing Purkinje neurons, but can also activate the Purkinje neuron-specific promoter, L7-pcp-2. Thus, in these cells the BMDC nucleus was reprogrammed after it fused to the Purkinje cell, enabling expression of the Purkinje-specific promoter L7-pcp-2. These results show that gene activation, only obtained previously in vitro in heterokaryons, can occur spontaneously in vivo. The results strongly suggest that the bone-marrow-derived nuclei are not only altered morphologically, but also reprogrammed in the adult Purkinje cell, as shown by the expression of the reporter gene GFP under the control of the L7-pcp-2 promoter.

These data show clearly that fusion is the underlying mechanism by which BMDCs contribute to Purkinje neurons. Fusion occurs spontaneously and physiologically to generate stable heterokaryons in the absence of selective pressure through genetic defects or drug treatment. The frequency increases over time, even though the blood-brain barrier opens only transiently. After a bone marrow transplant from a transgenic mouse ubiquitously expressing GFP, numerous GFP-positive cells were found to be binucleate Purkinje/BMDC heterokaryons in which the nuclei remained intact and distinct. Such heterokaryons increased in frequency with increasing age of the mouse. The morphology of the more than 300 GFP-positive cells analysed was typical of functional thriving Purkinje cells, with axons and full complex dendritic trees from which synaptic spines projected. Fusion of BMDC was specific to these cells, as no other neurons in this part of the brain expressed GFP after transplant. This finding is of particular interest, as Purkinje neurons are the most complex and elaborate in the cerebellum and have a critical function in balance and movement. Definitive proof that the binucleate cells resulted from fusion was obtained after transplantation of male bone marrow into female mice and detection of a Y chromosome in one of the two nuclei per heterokaryon.

To date, the only other example of BMDC fusion to tissue-specific cells in vivo is in the liver. Vassilopoulos, G., Wang, P. R. & Russell, D. W. Transplanted bone marrow regenerates liver by cell fusion. Nature 422, 901-904 (2003). Wang, X. et al. Cell fusion is the principal source of bone-marrow-derived hepatocytes. Nature 422, 897-901 (2003). The results of these studies are distinct from those reported here in several respects: first, although the initial frequency of fusion agrees with the data in this report (1/50,000), the hepatocyte/BMDC fusion product is not a stable heterokaryon. Instead, it proliferates extensively, resulting in millions of highly aneuploid progeny; second, the liver studies used strong selective pressure, the survival of a mouse with a lethal genetic disorder, tyrosinemia, as well as repeated drug administration. For survival, expansion of the rare BMDC/hepatocyte fusion events was absolutely necessary. The resulting karyotypic instability was presumably well tolerated because adult hepatocytes are typically multinuclear, polyploid and even aneuploid 37-39. As 6% of donor bone-marrow-derived hepatocytes were diploid 20, the possibility remains that a cell-fate change from BMDC to hepatocyte occurred before fusion, rather than after fusion with host hepatocytes. Thus, it is unclear which came first, a cell fate change or cell fusion.

In summary, the findings reported here are highly unexpected and significant for several reasons, including, for example, the following: heterokaryons formed spontaneously in vivo through the fusion of two disparate cell types, resulting in stably binucleate cells with equivalent chromosomal input. These data demonstrate that cell fusion in Purkinje neurons of the mouse brain can occur under physiological conditions without ongoing selective pressure. The result of this fusion is a heterokaryon containing a reprogrammed bone marrow nucleus, presumably through the increased dosage of regulatory proteins in the much larger Purkinje cell cytoplasm. Each GFP-positive fusion product, of the hundreds examined, was binucleate, and the frequency of this event increased with age.

Methods

Bone marrow transplantation. Marrow was isolated under sterile conditions from 8-10-week-old C57BL/6 transgenic mice that ubiquitously expressed enhanced green fluorescent protein (GFP)42. Donor mice were killed by cervical dislocation, briefly immersed in 70% ethanol and their skin peeled back from a midline, circumferential, incision. After the femurs, tibias and humeri were removed, all muscle was scraped away with a razor blade and the bones were placed in 10 ml of calcium and magnesium-free, Hank's balanced salt solution (HBSS, Invitrogen, Carlsbad, Calif.) with 2.5% foetal calf serum (FCS; SH30072.03, HyClone, Logan, Utah) on ice for up to 90 min. The tips of the bones were removed and a 25-gauge needle containing 1 ml of ice-cold HBSS with 2.5% FCS was inserted into the marrow cavity and used to wash the marrow out into a sterile culture dish. Marrow fragments were dissociated by triturating through the 25-gauge needle, and the resulting suspension was filtered through sterile 70 μm nitex mesh (BD-Falcon, Franklin Lakes, N.J.). The filtrate was cooled on ice, spun for 5 min at 250 g, and the pellet was resuspended in ice-cold HBSS with 2.5% FCS to 8×10⁷ nucleated cells per ml. Simultaneously, 8-10-week-old C57BL/6 mice (Stanford) were lethally irradiated with two doses of 4.8 Gy 3 h apart. Each irradiated recipient received 125 μl of the unfractionated marrow cell suspension by tail-vein injection within 1 h of the second irradiation dose.

Harvesting of brains. Mice were sacrificed at various times after bone marrow transplantation. The mice received a lethal injection of pentobarbital (Sleepaway, Fort Dodge Animal Health, Fort Dodge, Iowa) and were immediately perfused with ice-cold phosphate buffer (PB) followed by 4% paraformaldehyde in PB. The brains were then removed and cryoprotected in a 20% sucrose/PB solution overnight. Thick tissue sections (35-50 μm) for antibody staining, enumeration of donor derived cell number and nuclear content were obtained on a sliding microtome (SM2000R; Leica, Bannockburn, Ill.). Thin sections for FISH were made on a cryostat (CM3050S; Leica) at 10-12 μm and mounted on gelatin-coated slides (Goldseal, Portsmouth, N.H.).

Antibody Staining. Antibodies against GFP (mouse 1:1000; #A-11120; rabbit 1:2000; A-11122, Molecular Probes, Eugene, Oreg.), Calbindin (1:1000; C9848, Sigma, St Louis, Mo.), MAP2 (M2376; 1:100, Sigma), CD11b (1:100; #553308, BD Biosciences PharMingen, San Diego, Calif.), CD45 (1:200; #553076BD Biosciences PharMingen), F4/80 (#RM2900; 1:50, Caltag, Burlingame, Calif.), Iba 1 (1:1000, a gift from Y. Imai, National Institute of Neurosciences, Tokyo Japan), were applied for 12 h at 4° C. to the floating sections after pre-incubation in blocking solution for 2 h. When mouse or rabbit primary antibodies were used, anti-CD16/CD32 (1:200) was also included (#553142; BD Biosciences PharMingen). The sections were then incubated in appropriate secondary antibodies overnight at 4° C. The blocking solution contained 5% goat serum, 3% BSA and 0.3% Triton X-100.

FISH analysis. Thin sections (12 μm) of the cerebella from GFP-transplanted mice were processed for GFP using standard immunohistochemistry. The nuclei were then counterstained with To-Pro3. These sections were then viewed for the presence of GFP-positive Purkinje neurons and scanned at 1 μm optical section using a scanning confocal microscope (LSM510; Zeiss, Thornwood, N.Y.). The x- and y-position of the GFP-positive cell bodies were recorded with respect to the corners of the slide, to relocate the exact position after FISH. The FISH protocol was modified from ref. 43 and protocols from Applied Spectral Imaging (Carlsbad, Calif.). Briefly, sections were then dehydrated, treated with proteinase K at 45° C. for 7-15 min, rinsed in 2×SSC and denatured in 70% formamide in 2×SSC at 68° C. for 5 min. The slides were then dehydrated and warmed to 50° C. The X and Y chromosome probes were denatured and applied as directed (see CamBio and Applied Spectral Imaging website). After 36 h at 37° C., the probe was washed off in 2×SCC, before incubation in 2×SSC/0.1% NP40 at 50° C. for 2 min and mounted with Vysis DAPI mounting solution with 1:3000 To-Pro3.

Flow cytometry and FACS Analysis. Bone marrow was prepared as described above, with the exception that erythrocytes were lysed in lysis buffer (0.15 M NH4Cl, 1.0 mM KHCO3 and 0.1 mM Na2EDTA at pH 7.4) for 5 min on ice before incubation with propidium iodine (PI; final concentration 100 g ml-1) to exclude dead cells. Total unfractionated bone marrow (100 μl) from five L7/GFP-Pcp-2 transgenic, one GFP-transgenic and three wild-type mice, respectively, were used to acquire data to determine whether bone marrow cells (one million cells) expressed GFP, using a FACSCalibur (BD Biosciences, San Diego, Calif.). These experiments were repeated in triplicate. Data were analysed and presented with FlowJo v.4.3 software (Tree Star, Inc., Ashland, Calif.), displayed as a contour plot at 5% probability, as a function of side scattered versus GFP fluorescence. All animals were processed simultaneously.

### Example 8

### Normal Tissue Maintenance Creates Differences in Regenerative Capacity

As described above, the PC is a muscle having particularly high incorporation of CSCs. Applicants have demonstrated that PC also has a higher frequency of myf-5 expressing satellite cells relative to other muscles, such as the TA. Myf-5 is a marker of satellite cells, and is generally considered to indicate that the satellite cell is activated (i.e., preparing to contribute to a mature myocyte).

Tissue sections were examined from the (a) tibialis anterior and (c) PC from a non-transplanted transgenic mouse in which the expression of the LacZ gene is regulated by the satellite cell specific promoter for the Myf-5 gene. The frequency of myf-5 expressing satellite cells was dramatically higher in the PC under normal physiological conditions than in, for example, the tibialis anterior. Thus, irradiation or other types of injury are not responsible for the high rate of regeneration seen in this muscle and other factors, such as normal tissue maintenance may cause higher regenerative capacity in certain tissues.

### Example 9

### Alloimmune Injury as a Form of Damage Associated with Increased BMDSC Regeneration

Chronic airway rejection, termed obliterative bronchiolitis (OB), is a fibroproliferative, inflammatory lung disease which results in obliteration of small airway lumens. Thus, this type of chronic rejection lesion is the result of an uncontrolled migration and proliferation of mesenchymal cells followed by connective tissue deposition.

Applicants and others have developed a novel model of OB in which tracheas heterotopically implanted into the greater omentum of major-histocompatability-complex (MHC) mismatched mice or rats develop an obliterative lesion in 28 days that is histologically similar to that seen in OB. Applicants call the development of this lesion in rodents obliterative airway disease (OAD). At the time of our research, the accepted etiology of chronic rejection was that the proliferation of mesenchymal cells responsible for the airway obliteration was a consequence of the proliferation of local fibroblasts in the airway wall adjacent to the lesion site. However, this explanation had never been directly tested but only hypothesized from the accumulated data.

Therefore Applicants used these rodent models to identify the origin of the proliferating mesenchymal cells and OB, to characterize the temporal pattern of recipient and donor cell infiltration and proliferation, and to evaluate the effects of immunosuppressive therapies on this process.

Materials and Methods

Animals: Male, viral-antibody-free, 6-8 week old, Brown Norway [BN] (RT1.Aⁿ) and Lewis [LEW] (RT1.A^{l}) rats were obtained (Charles Rivers Laboratories). Brown Norway trachea were implanted into the omentum of isogeneic Brown Norway rats or allogeneic Lewis rats for 28 days. C57B/6 ROSA26 were obtained from Jackson laboratories mice and wildtype C57B/6 and BALB/c mice were obtained from Stanford's colony. Bone marrow was harvested from adult ROSA26 mice that ubiquitously express B-gal and transplanted into lethally irradiated isogeneic recipients. Two months after bone marrow transplantation, wild type C57B/6 or BALB/c tracheae were implanted into the omentum of BMT recipients for 28 days.

Surgical procedure: Tracheae were heterotopically grafted into the greater omentum of recipients. All procedures were completed under general anesthesia. Briefly, the donor trachea was sectioned just distal to the cricoid cartilage and just proximal to the bronchial bifurcation. Following tracheal harvest, donors were euthanized by cervical dislocation under anesthesia. The resulting 1 cm tracheal segment was removed and placed in ice-cold, sterile, PhysioSol (Abbot Laboratories) for a maximum of 15 minutes. In the recipient, the greater omentum was exposed by a 2 cm midline laparotomy. One donor trachea was individually wrapped in the omentum and secured by two 6-0 Prolene sutures. Abdominal wall and skin were individually closed by standard surgical procedures using 4-0 absorbable suture.

Bone marrow transplantation: Bone marrow was harvested from 8-10 week old, male ROSA26 mice that ubiquitously expressed B-gal. Briefly, donor mice were euthanized by cervical dislocation, immersed in 70% ethanol, and the skin was peeled back from a midline, circumferential incision. Large limb bones (femur, tibia, & humerus) were surgically isolated and placed in ice-cold of calcium and magnesium-free, Hank's balanced salt solution (HBSS, Irvine Scientific) with 2% FBS for up to 90 minutes. In a tissue culture hood, the tips of the bones were removed and a 25 gauge needle containing 1 mL of ice-cold HBSS with 2% FCS was inserted into the marrow cavity and used to wash the marrow out into a sterile culture dish. Marrow fragments were dissociated by titurating through the 25 gauge needle and the resulting suspension was filtered through sterile 70 μm nitex mesh. The filtrate was cooled on ice, spun for 5 minutes at 250×g, and the pellet was resuspended in ice-cold HBSS with 2% FCS to 4.8×10⁷ nucleated cells per mL.

Graft removal: After 28 days tracheal grafts were harvested, fixed in phosphate buffered formalin, embedded in OCT media, snap frozen in liquid nitrogen, and stored at −80° C. for later cryosectioning and immunohistochemistry. All histological sections were cut from the areas corresponding to what had been the central portion of the original tracheal segments.

Immunohistochemistry: To identify the donor or recipient origin of cells in rat tissue, tracheal sections were stained with monoclonal antibodies (mAB) specific for BN (mAB 42-3-7) or LEW (mAB 163-7F3) major histocompatibility complex class I antigens (MHC I). In detail, OCT-embedded 6 μm frozen tracheal tissue sections were air-dried and fixed in acetone at −20° C. for 10 min and washed for 10 min in phosphate-buffered saline. The sections were then incubated at room temperature for 30 min with a biotinylated antibody diluted 1:120 for anti-BN staining and 1:100 for anti-Lewis staining, and washed 10 minutes in phosphate-buffered saline. Slides were then incubated with FITC-streptavidin conjugate (Boehringer Mannheim) at 2.5 μg/mL or 5 μg/mL (anti-BN and anti-LEW staining, respectively) and mounted with p-phenyl-diamine medium. For positive and negative controls, sections from native Lewis tracheas, and native BN tracheas were stained with both antibodies. Both negative controls (anti-BN staining of Lewis tissue and anti-Lewis staining of BN tissue) had minimal background staining while positive controls (anti-Lewis staining of Lewis tissue and anti-BN staining of BN tissue) demonstrated a moderate staining of all cell types. The number of positive cells was rated 0-4 (0=no positive cells, 4=uniform and exclusive infiltrate of positive cells).

Cellular infiltrates were characterized with antibody clones W3/25 (detects rat equivalent of CD4), MRC OX8 (CD8), R73, (α/β T cell receptor), MRC OX33 (CD45RA, present on most B cells), ED1 (monocytes and macrophages), ED2 (macrophages), and OX3 and OX6 (MHC II expression). All antibodies were from Serotec (Accurate Antibodies), San Diego, Calif. Tracheal sections embedded in OCT were brought to −20° C. and 5 μm thin sections were placed onto poly-L-lysine pre-coated slides (Cat# P-0425, Sigma Diagnostics, St-Louis Mo.). Sections were air dried at room temperature, fixed in acetone at −20° C. overnight, rehydrated in PBS for 10 minutes, incubated with primary antibody diluted optimally in 5% FCS in PBS for 30 minutes, washed with PBS, and incubated with a rabbit anti-mouse secondary antibody conjugated with horseradish peroxidase. Sections were developed with 3,3′-diaminobenzidine tetrahydrochloride in 0.05 M Tris buffer and 0.015% H₂O₂ until staining intensity was optimized. Sections were semiquantitatively scored by an observer who was blinded to the experimental groups on a scale from 0-3 (0=no infiltrate, 3=dense infiltrate).

Results

(i) Development of OAD in Allografts but not Isografts

Untreated rat tracheal isografts (BN or LEW) heterotopically implanted into the greater omentum of isogeneic recipients did not develop luminal obliteration (mean luminal obliteration of 0% for both). Mononuclear inflammatory cells were sparse or absent in all tracheae, and neither fibrosis nor spindled cells were observed. Granulation tissue was minimal in 4/10 tracheae and absent in 6/10. All tracheae were lined by respiratory epithelium.

In contrast, untreated BN rat tracheal allografts heterotopically implanted into the greater omentum of LEW recipients developed OAD lesions with a median of 100% obliteration by day 28. The OAD lesions observed in day 28 allografts consisted of a uniform, edematous fibroconnective tissue stroma with a mild to moderate infiltration of mononuclear cells. The degree of fibrosis within the lumens as demonstrated by Mason's trichrome stain was mild to moderate (mean: 2.8) and early collagen tissue was distributed uniformly throughout the lesion. The predominant cells were spindle cells resembling fibroblasts and myofibroblasts. The mononuclear cell infiltration was less dense than that observed on days 14 and 21 and was evenly distributed between luminal and peritracheal tissue. Overall, this OAD lesion was histologically very similar to that seen in clinical OB.

Each anti-MHC-I-antibody specifically stained smooth muscle, fibroblast, mononuclear, epithelial and endothelial cells of isografts from the corresponding rat strain only. In allografted BN tracheas, a progressive infiltration of mononuclear LEW cells was observed on days 3 and 7. By day 14, infiltrating LEW mononuclear and mesenchymal cells began to enter the tracheal lumen. The temporal and spatial pattern of infiltration by LEW-type mononuclear cells was similar to that of CD8+ or CD4+ T cells, but not monocytic, myeloid, or B cells. By days 21 and 28, all cells within the obliterative airway lesion were of LEW origin, with the exception of the remnants of the luminal basal lamina. This infiltration of LEW mononuclear and mesenchymal cells that resulted in the luminal obliteration was confirmed by a failure of this tissue to stain with the antibody against donor MHC I.

While the intent of this study was to determine the origin of the mesenchymal cells involved in the formation of the OAD lesion, Applicants made the unexpected observation that a dramatic degree of tissue remodeling and replacement occurred within the tracheal allografts. Specifically, the tracheal pericartilagenous tissue, including smooth muscle cells and fibroblasts, progressively transformed from a BN to a LEW phenotype during the allograft period without a visible disruption of tissue organization by standard light microscopy. This loss of donor tissue was first evident at the tracheal periphery by day 7. The loss of donor cells gradually progressed from the periphery toward the center so that by day 14 there was a dramatic loss of cells that had constituted the original tracheal graft and by day 21 donor-type cells were essentially absent from the tracheal grafts (with the exception of the remnants of the basal lamina and chondrocytic cells).

(ii) Early Characterization of Infiltrating LEW Mesenchymal Cells

Additional tissue sections were stained with antibodies to identify T, CD4+, CD8+, and B cells, as well as monocytes and macrophages. The temporal and spatial pattern of infiltration by LEW-type mononuclear cells was similar to that of CD8+ T cells and monocytes at early but not late stages and similar to CD8+ T cells at later stages. The pattern of infiltration was not similar to infiltrations by CD4+ T, monocytic, myeloid, or B cells.

A Subset of Infiltrating Recipient Cells Arise from BMDSC

Analysis of allografts that had been implanted into mice that had received a bone marrow transplant with ROSA 26 bone marrow, revealed that approximately 30% of fibroblasts in both the interluminal lesion and in the pericartilagenous tissue expressed B-gal, indicating that they were the progeny of the transplanted population of bone marrow cells.

### Example 10

### Adult Hematopoietic Stem Cell Populations Contain Skeletal Myofiber Progenitors

Methods

Specific populations of BM were selected by incubating the cells with antibody cocktails and then enriching for specific populations on a fluorescent activated cell sorter (FACS). Hematopoietic lineage cells were depleted with a panel of biotin-labeled antibodies (2 μL of each antibody/1E6 cells, BD Biosciences) consisting of anti-CD3e, anti-CD11b, anti-CD45R/B220, anti-Ly-6G, and anti-TER-119 which were detected with streptavidin-Texas Red (3 μg/1E6 cells; Molecular Probes). Specific populations were selected using anti-c-kit, anti-Sca-1, anti-CD38, and anti-CD34 (all four from Pharmingen, 1.5 μg/1E6 cells). The marrow of 8-10 week old, isogeneic (C57B/6, Stanford), recipient mice was ablated by lethal irradiation (two doses of 475 cGy, three hours apart) after which each mouse received selected cell populations by tail vein injection. Several mice that received selected populations of GFP+ BM required irradiated transfusions of unlabeled (GFP-negative) platelets and/or red blood cells in the 2-3 weeks immediately post-transplant.

Generation of single SPKLS cell-reconstituted mice. Isolation of SPKLS cells: Approximately 30% of the Lineage negative, Hoechst dim (SP) cells are c-kit and Sca-1 positive. Cells falling in the gates shown were isolated by flow cytometry and clonally re-sorted in 96 well plates. Wells containing single cells were identified by inspection with both GFP and Hoechst filters. The kinetics of single-cell reconstitution in a representative experiment (Exp. 3 from supplementary online Table 1). Animals displaying long term (over 16 weeks) contribution of GFP-positive cells to all blood lineages were chosen for further analysis. Repopulation of the individual lineages was assessed by staining BM and peripheral blood for B cells (B220), T cells (CD3), granulocytes (GR-1) and monocytes (Mac-1).

Detection of spontaneously arising myofibers in the Panniculus Carnosus (PC) muscle. The readily accessible location of the PC facilitates the identification of GFP+ myofibers by whole mount microscopy. The entire pelt was removed and spread onto a glass plate to image the muscle layer. The characteristic laminin-rich basal membrane surrounding the myofibers and the localization of GFP according to a sarcomeric pattern were detected by confocal microscopy. These criteria were used to unambiguously verify the identity of the GFP+ structures detected by the whole mount method.

Identification of BM-derived myofibers in single cell-reconstituted animals. GFP+ myofibers were visualized in the PC. The characteristic sarcomeric pattern was evident in optical sections generated by confocal microscopy. Three dimensional projection of a stack of 95 serial optical sections showed a GFP+ myotube crossing the thickness of a cryosection. Basal membrane surrounds GFP+ structures in the PC, which is characteristic of myofibers. Confocal images of GFP+ myofibers, and the surrounding laminin sheath were captured four weeks after Notexin injection. In Notexin treated samples, several myofibers appeared faintly positive for GFP both by confocal analysis and by epifluorescence using a LP510 filter on the emission path. Use of this long pass filter allowed the distinction between true GFP fluorescence, which appears green, and autofluorescence, which appears yellow. While applicants believe these may represent myofibers with a low level of integration of circulating cells, possibly containing only one BM-derived nucleus, only unambiguously positive myofibers were counted and reported in the Table 1.

BM derived myofibers in secondary recipients. Analysis of the peripheral blood of secondary recipients showed multilineage engraftment, proving that the original SPKLS cells was capable of self renewal. The animals were analyzed four months after transplantation with total bone marrow from a single cell repopulated mouse.

Results

Four populations of marrow from GFP+mice were selected by FACS. All sorted populations excluded cells expressing one or more lineage specific proteins [Lin(−)]. The lineage-specific proteins used to deplete mature cells were CD3e (found on thymocytes and mature T cells), CD11b (granulocytes, monocytes/macrophages, dendritic cells, natural killer cells, and B-1 cells), CD45R/B220 (all B lineage cells and on peripheral NK and CTL cells), Ly-6G (granulocytes), and anti-TER-119 (erythroid cells).

All four selected populations allowed full hematopoietic reconstitution of recipient mice with GFP+ cells by eight weeks post-transplant. When mice were harvested at seven months post-transplant, only three selected populations were found that contained the capacity to generate skeletal myofibers in the PC. Two of these populations were the Sca-1+, c-kit+, Lin- and Sca-1+, c-kit(−), and Lin(−) populations. The third population with myofiber regenerative capacity has been termed the side population (SP) and is identified by the differential retention of the DNA-binding Hoechst 33342 dye in the presence of the drug, verapamil, which blocks dye efflux. Interestingly, the number of myofibers generated in the PC by these sorted populations was indistinguishable from that generated by whole BM, suggesting that all three of these populations contained a sufficient capacity to generate skeletal myofibers to meet regenerative requirements, at least for period of time in our study.

The fourth sorted population, which was CD34(−), CD38(−), Lin(−), failed to generate any skeletal myofibers despite the full hematopoietic reconstitution of recipient at both eight weeks post-transplant and at the time of tissue harvest. It is unclear why CD34(−), CD38(−) cells failed to generate skeletal myofibers.

To identify unambiguously the source of myogenic cells, Applicants focused on the subset of the Side Population that is c-Kit+, Lineage negative (Lin-), Sca-1+(SPKLS). This fraction represents approximately 0.01% of all bone marrow cells and is highly enriched in hematopoietic stem cells. Double-sorted SPKLS cells from a GFP+CD45.1 C57/B6 mouse strain were clonally deposited in the wells of 96 well plates. The plates were then inspected by fluorescence microscopy with both GFP and Hoechst filters, and wells containing only a single cell were selected. These single cells were then transplanted into irradiated CD45.2 congenic recipients together with 10⁶ GFP-negative CD45.1 BM cells depleted of repopulating hematopoietic stem cells. Approximately 30% of the recipients developed significant levels of GFP+ peripheral blood four weeks after transplant. Mice with greater than 30% of the peripheral blood originating from the single transplanted cell and displaying multi-lineage engraftment for more than 16 weeks were analyzed for the presence of GFP+ myofibers.

The presence of GFP-positive myofibers was scored in two different muscle, the undamaged Panniculus Carnosus and the toxin-damaged Tibialis Anterior (TA).

In one of the six single cell-reconstituted mice analyzed, 31 GFP positive myofibers were identified in the PC by whole mount fluorescence microscopy revealing a robust contribution to myogenesis. Immunofluorescent staining for laminin encasing the myofibers as well as the detection of the characteristic sarcomeric pattern was used to confirm the identity of these cells. These results suggest that hematopoietic stem cells can efficiently contribute to skeletal muscle in the absence of local, experimentally-induced damage.

The determine whether muscle damage can increase the recruitment of circulating cells into myofibers, applicants injected the myotoxin Notexin in the Tibialis Anterior (TA) muscles of 3 single cell reconstituted mice in which the blood had been reconstituted by a single cell. The first animal was sacrificed too soon after injection, a time when the inflammatory response to local damage was maximal and precluded quantification (mouse #4, Table 1). The remaining two animals were sacrificed one month after Notexin injection. In these two animals a number of morphologically normal, GFP+ myofibers were readily detected in the area surrounding the damage, but not in the contralateral TA or in the undamaged PC. Thus, local damage leads to the integration of circulating cells into regenerating myofibers in all the animals analyzed.

Hematopoietic stem cells are defined by their ability to engraft and yield multi-lineage repopulation in secondary recipients. When secondary transplants were performed with total bone marrow harvested from a single cell repopulated animal (Table 1, mouse #2), all of the peripheral blood lineages in the secondary recipients were found to contain GFP-positive cells for up to four months after transplant, indicating that the original cell was capable of self-renewal. Although applicants were not able to detect GFP-positive myofibers in the PC of this particular primary recipient, in one of the three secondary recipients derived from it, the PC contained GFP-positive myofibers. This finding together with the observation that local damage readily induced the formation of GFP-positive myofibers in primary recipients, suggests that the observation of spontaneously arising GFP-positive myofibers in only one of six mice analyzed likely reflects the requirement for specific microenvironmental conditions, such as an increased regeneration rate in the PC, rather than an intrinsic propensity of the transplanted cells to participate in muscle regeneration.

### REFERENCES

All publications and patents mentioned herein are hereby incorporated by reference in their entirety as if each individual publication or patent was specifically and individually indicated to be incorporated by reference. In case of conflict, the present application, including any definitions herein, will control.
Anderson, D. J., Gage, F. H., and Weissman, I. L. (2001). Can stem cells cross lineage boundaries?, Nat Med 7, 393-5.
Bao, Z. Z., Lakonishok, M., Kaufman, S., and Horwitz, A. F. (1993). Alpha 7 beta 1 integrin is a component of the myotendinous junction on skeletal muscle, J Cell Sci 106, 579-89.
Barofflo, A., Hamann, M., Bernheim, L., Bochaton-Piallat, M. L., Gabbiani, G., and Bader, C. R. (1996). Identification of self-renewing myoblasts in the progeny of single human muscle satellite cells, Differentiation 60, 47-57.
Beauchamp, J. R., Heslop, L., Yu, D. S., Tajbakhsh, S., Kelly, R. G., Wernig, A., Buckingham, M. E., Partridge, T. A., and Zammit, P. S. (2000). Expression of CD34 and Myf5 defines the majority of quiescent adult skeletal muscle satellite cells, J Cell Biol 151, 1221-34.
Beauchamp, J. R., Morgan, J. E., Pagel, C. N., and Partridge, T. A. (1999). Dynamics of myoblast transplantation reveal a discrete minority of precursors with stem cell-like properties as the myogenic source, J Cell Biol 144, 1113-22.
Bischoff, R. (1986). Proliferation of muscle satellite cells on intact myofibers in culture, Developmental Biology 115, 129-139.
Bittner, R. E., Schofer, C., Weipoltshammer, K., Ivanova, S., Streubel, B., Hauser, E., Freilinger, M., Hoger, H., Elbe-Burger, A., and Wachtler, F. (1999). Recruitment of bone-marrow-derived cells by skeletal and cardiac muscle in adult dystrophic mdx mice, Anat Embryol (Berl) 199, 391-6.
Blanco-Bose, W. E., Yao, C. C., Kramer, R. H., and Blau, H. M. (2001). Purification of mouse primary myoblasts based on alpha 7 integrin expression, Exp Cell Res 265, 212-20.
Blaveri, K., Heslop, L., Yu, D. S., Rosenblatt, J. D., Gross, J. G., Partridge, T. A., and Morgan, J. E. (1999). Patterns of repair of dystrophic mouse muscle: studies on isolated fibers, Dev Dyn 216, 244-56.
Brazelton, T. R., Rossi, F. M., Keshet, G. I., and Blau, H. M. (2000). From marrow to brain: expression of neuronal phenotypes in adult mice, Science 290, 1775-9.

Castro, R. F., Jackson, K. A., Goodell, M. A., Robertson, C. S., Liu, H., and Shine, H. D. (2002). Failure of bone marrow cells to transdifferentiate into neural cells in vivo, Science 297, 1299.
Cornelison, D. D., and Wold, B. J. (1997). Single-cell analysis of regulatory gene expression in quiescent and activated mouse skeletal muscle satellite cells, Dev Biol 191, 270-83.
Cossu, G., Tajbakhsh, S., and Buckingham, M. (1996). How is myogenesis initiated in the embryo?, Trends Genet 12, 218-23.
Ferrari, G., Cusella-De Angelis, M. G., Coletta, M., Paolucci, E., Stornaiuolo, A., Cossu, G., and mavilio, F. (1998). Muscle regeneration by bone marrow-derived myogenic progenitors, Science 279, 1528-1530.
Ferrari, G., Stornaiuolo, A., and Mavilio, F. (2001). Failure to correct murine muscular dystrophy, Nature 411, 1014-5.
Fukada, S., Miyagoe-Suzuki, Y., Tsukihara, H., Yuasa, K., Higuchi, S., Ono, S., Tsujikawa, K., Takeda, S., and Yamamoto, H. (2002). Muscle regeneration by reconstitution with bone marrow or fetal liver cells from green fluorescent protein-gene transgenic mice, J Cell Sci 115, 1285-93.
Goyer, R. A., and Yin, M. W. (1967). Taurine and creatine excretion after x-irradiation and plasmocid-induced muscle necrosis in the rat, Radiat Res 30, 301.
Grounds, M. D. (1998). Age-associated changes in the response of skeletal muscle cells to exercise and regeneration, Ann N Y Acad Sci 854, 78-91.
Grounds, M. D. (1999). Muscle regeneration: molecular aspects and therapeutic implications, Current opinion in neurology 12, 535-543.
Gulati, A. K. (1987). The effect of X-irradiation on skeletal muscle regeneration in the adult rat, J Neurol Sci 78, 111-20.
Gussoni, E., Soneoka, Y., Strickland, C. D., Buzney, E. A., Khan, M. K., Flint, A. F., Kunkel, L. M., and Mulligan, R. C. (1999). Dystrophin expression in the mdx mouse restored by stem cell transplantation, Nature 401, 390-4.
Heslop, L., Morgan, J. E., and Partridge, T. A. (2000). Evidence for a myogenic stem cell that is exhausted in dystrophic muscle, J Cell Sci 113, 2299-308.
Hughes, S. M., and Blau, H. M. (1990). Migration of myoblasts across basal lamina during skeletal muscle development, Nature 345, 350-3.
Jackson, K. A., Majka, S. M., Wang, H., Pocius, J., Hartley, C. J., Majesky, M. W., Entman, M. L., Michael, L. H., Hirschi, K. K., and Goodell, M. A. (2001). Regeneration of ischemic cardiac muscle and vascular endothelium by adult stem cells, J Clin Invest 107, 1395-402.
Kadi, F., and Thornell, L. E. (2000). Concomitant increases in myonuclear and satellite cell content in female trapezius muscle following strength training, Histochem Cell Biol 113, 99-103.
Korbling, M., Katz, R. L., Khanna, A., Ruifrok, A. C., Rondon, G., Albitar, M., Champlin, R. E., and Estrov, Z. (2002). Hepatocytes and epithelial cells of donor origin in recipients of peripheral-blood stem cells, N Engl J Med 346, 738-46.
Kornack, D. R., and Rakic, P. (2001). Cell proliferation without neurogenesis in adult primate neocortex, Science 294, 2127-30.
Krause, D. S., Theise, N. D., Collector, M. I., Henegariu, O., Hwang, S., Gardner, R., Neutzel, S., and Sharkis, S. J. (2001). Multi-organ, multi-lineage engraftment by a single bone marrow-derived stem cell, Cell 105, 369-77.
Lagasse, E., Connors, H., Al-Dhalimy, M., Reitsma, M., Dohse, M., Osborne, L., Wang, X., Finegold, M., Weissman, I. L., and Grompe, M. (2000). Purified hematopoietic stem cells can differentiate into hepatocytes in vivo, Nat Med 6, 1229-34.
Mauro, A. (1961). Satellite cells of muscle skeletal fibers, J Biophys Biochem 9, 493-495.
Mezey, E., Chandross, K. J., Harta, G., Maki, R. A., and McKercher, S. R. (2000). Turning blood into brain: cells bearing neuronal antigens generated in vivo from bone marrow, Science 290, 1779-82.
Okabe, M., Ikawa, M., Kominami, K., Nakanishi, T., and Nishimune, Y. (1997). ‘Green mice’ as a source of ubiquitous green cells, FEBS Lett 407, 313-9.
Orlic, D., Kajstura, J., Chimenti, S., Jakoniuk, I., Anderson, S. M., Li, B., Pickel, J., McKay, R., Nadal-Ginard, B., Bodine, D. M., et al. (2001). Bone marrow cells regenerate infarcted myocardium, Nature 410, 701-5.
Quaini, F., Urbanek, K., Beltrami, A. P., Finato, N., Beltrami, C. A., Nadal-Ginard, B., Kajstura, J., Leri, A., and Anversa, P. (2002). Chimerism of the transplanted heart, N Engl J Med 346, 5-15.
Rando, T. A., and Blau, H. M. (1994). Primary mouse myoblast purification, characterization, and transplantation for cell-mediated gene therapy, J Cell Biol 125, 1275-87.
Rosenblatt, J. D., Lunt, A. I., Parry, D. J., and Partridge, T. A. (1995). Culturing satellite cells from living single muscle fiber explants, In Vitro Cell Dev Biol Anim 31, 773-9.
Rosenblatt, J. D., Yong, D., and Parry, D. J. (1994). Satellite cell activity is required for hypertrophy of overloaded adult rat muscle, Muscle Nerve 17, 608-13.
Schofield (1978). The relationship between the spleen colony forming cell ant the haematopoietic stem cell, Blood Cells 4, 7-25.
Spradling (2001). Stem cells find their niche, Nature 414, 98-104.
Terada, N., Hamazaki, T., Oka, I., Hoki, M., Mastalerz, D. M., Nakano, Y., Meyer, E. M., Morel, L., Petersen, B. E., and Scott, E. W. (2002). Bone marrow cells adopt the phenotype of other cells by spontaneous cell fusion, Nature 416, 542-545.
Theise, N. D., Badve, S., Saxena, R., Henegariu, O., Sell, S., Crawford, J. M., and Krause, D. S. (2000). Derivation of hepatocytes from bone marrow cells in mice after radiation-induced myeloablation, Hepatology 31, 235-40.
Wagers, A. J., Sherwood, R. I., Christensen, J. L., and Weissman, I. L. (2002). Little evidence for developmental plasticity of adult hematopoietic stem cells, Science 297, 2256-9.
Wang, X., Montini, E., Al-Dhalimy, M., Lagasse, E., Finegold, M., and Grompe, M. (2002). Kinetics of liver repopulation after bone marrow transplantation, Am J Pathol 161, 565-74.
Warren, S. (1943). Effects of radiation on normal tissues. XIV. Effects on striated muscle, Arch Path 35, 347.
Watt, F. M., and Hogan, B. L. (2000). Out of Eden: stem cells and their niches, Science 287, 1427-30.
Ying, Q., Nichols, J., Evans, E. P., and Smith, A. G. (2002). Changing potency by spontaneous fusion, Nature 416, 545-548.
Zammit, P., and Beauchamp, J. (2001). The skeletal muscle satellite cell: stem cell or son of stem cell?, Differentiation 68, 193-204.
Blau, H. M., Brazelton, T. R. & Weimann, J. M. The evolving concept of a stem cell: entity or function? Cell 105, 829-41. (2001).
Priller, J. et al. Neogenesis of cerebellar Purkinje neurons from gene-marked bone marrow cells in vivo. J Cell Biol 155, 733-8. (2001).
Ferrari, G. et al. Muscle regeneration by bone marrow-derived myogenic progenitors. Science 279, 1528-30. (1998).
Gussoni, E. et al. Dystrophin expression in the mdx mouse restored by stem cell transplantation. Nature 401, 390-4. (1999).
Krause, D. S. et al. Multi-organ, multi-lineage engraftment by a single bone marrow-derived stem cell. Cell 105, 369-77. (2001).
Orlic, D. et al. Bone marrow cells regenerate infarcted myocardium. Nature 410, 701-5. (2001).
Petersen, B. E. et al. Bone marrow as a potential source of hepatic oval cells. Science 284, 1168-70. (1999).
Korbling, M. et al. Hepatocytes and epithelial cells of donor origin in recipients of peripheral-blood stem cells. N Engl J Med 346, 738-46. (2002).
Okamoto, R. et al. Damaged epithelia regenerated by bone marrow-derived cells in the human gastrointestinal tract. Nat Med 8, 1011-7. (2002).
Theise, N. D. et al. Derivation of hepatocytes from bone marrow cells in mice after radiation-induced myeloablation. Hepatology 31, 235-40. (2000).
Alison, M. R. et al. Hepatocytes from non-hepatic adult stem cells. Nature 406, 257. (2000).
Castro, R. F. et al. Failure of bone marrow cells to transdifferentiate into neural cells in vivo. Science 297, 1299. (2002).
Wagers, A. J., Sherwood, R. I., Christensen, J. L. & Weissman, I. L. Little evidence for developmental plasticity of adult hematopoietic stem cells. Science 297, 2256-9. (2002).
Blau, H., Brazelton, T., Keshet, G. & Rossi, F. Something in the eye of the beholder. Science 298, 361-2; discussion 362-3. (2002).
Munz, B., Wiedmann, M., Lochmuller, H. & Werner, S. Cloning of novel injury-regulated genes. Implications for an important role of the muscle-specific protein skNAC in muscle repair. J Biol Chem 274, 13305-10. (1999).
Cho, M., Webster, S. G. & Blau, H. M. Evidence for myoblast-extrinsic regulation of slow myosin heavy chain expression during muscle fiber formation in embryonic development. J Cell Biol 121, 795-810. (1993).
Hughes, S. M. & Blau, H. M. Muscle fiber pattern is independent of cell lineage in postnatal rodent development. Cell 68, 659-71. (1992).
Hughes, S. M. et al. Three slow myosin heavy chains sequentially expressed in developing mammalian skeletal muscle. Dev Biol 158, 183-99. (1993).
Webster, C., Silberstein, L., Hays, A. P. & Blau, H. M. Fast muscle fibers are preferentially affected in Duchenne muscular dystrophy. Cell 52, 503-13. (1988).
Sanes, J. R. & Lichtman, J. W. Development of the vertebrate neuromuscular junction. Annu Rev Neurosci 22,389-442 (1999).
Grounds, M., Partridge, T. A. & Sloper, J. C. The contribution of exogenous cells to regenerating skeletal muscle: an isoenzyme study of muscle allografts in mice. J Pathol 132, 325-41. (1980).
Heslop, L., Morgan, J. E. & Partridge, T. A. Evidence for a myogenic stem cell that is exhausted in dystrophic muscle. J Cell Sci 113, 2299-308. (2000).
Wright, A. E. Medical Physics Handbook of Radiation Therapy, (Medical Physics Publ., Madison, Wis., 1992).
Grounds, M. D. Age-associated changes in the response of skeletal muscle cells to exercise and regeneration. Ann N Y Acad Sci 854, 78-91. (1998).
Okabe, M., Ikawa, M., Kominami, K., Nakanishi, T. & Nishimune, Y. ‘Green mice’ as a source of ubiquitous green cells. FEBS Letters 407, 313-9 (1997).
E. Gussoni et al., Nature 401, 390-4 (1999).
G. Ferrari et al., Science 279, 1528-30 (1998).
M. A. LaBarge, H. M. Blau, Cell 111, 589-601 (2002).
R. E. Bittner et al., Anat Embryol (Berl) 199, 391-6 (1999).
E. Gussoni et al., The Journal of clinical investigation. 110(6), 807-14 (2002).
D. G. Baker, R. J. Krochak, Cancer Invest 7, 287-94 (1989).
J. L. Ferrara, Curr Opin Immunol 5, 794-9 (1993).
M. D. Grounds, J. D. White, N. Rosenthal, M. A. Bogoyevitch, J Histochem Cytochem 50, 589-610 (2002).
J. B. Harris, M. A. Johnson, Clin Exp Pharmacol Physiol 5, 587-600 (1978).
C. Klein-Ogus, J. B. Harris, Cell Tissue Res 230, 671-6 (1983).
C. R. Slater, E. G. Allen, J Physiol (Paris) 80, 238-46 (1985).
T. R. Brazelton, M. Nystrom, H. M. Blau, Dev Biol 262, 64-74 (2003).
S. Y. Corbel et al., Nat Med in press (2003).
G. Zhou et al., Proc Natl Acad Sci USA 98, 13966-71 (2001).
M. Ogawa et al., J Exp Med 174, 63-71 (1991).
M. van de Rijn, S. Heimfeld, G. J. Spangrude, I. L. Weissman, Proc Natl Acad Sci U S A 86, 4634-8 (1989).
A. J. Wagers, R. I. Sherwood, J. L. Christensen, I. L. Weissman, Science 297, 2256-9 (2002).
A. Polesskaya, P. Seale, M. A. Rudnicki, Cell 113, 841-52 (2003).
M. Sampaolesi et al., Science 301, 487-92 (2003).
   Equivalents

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

We claim:

1. A method for assessing the ability of a test treatment to alter the contribution of a stem cell to a target tissue in a subject, the method comprising:
a) administering the test treatment to the subject;
b) detecting the contribution of a stem cell to the target tissue;
wherein the stem cell is of a distinct developmental lineage from the target tissue.

2. The method of claim 1, further comprising comparing the detected contribution to a reference.

3. The method of claim 2, wherein the reference is a measure of contribution of stem cells to the target tissue in the absence of the test treatment.

4. The method of claim 1, wherein the stem cell is a circulating stem cell.

5. The method of claim 4, wherein circulating stem becomes, or fuses with, a tissue-localized stem cell.

6. The method of claim 1, wherein the test treatment comprises the administration of a test agent.

7. The method of claim 4, wherein the test agent is a polypeptide.

8. The method of claim 4, wherein the administering the agent comprises administering a recombinant nucleic acid that encodes the test agent.

9. The method of claim 8, wherein the test agent is selected from the group consisting of: a polypeptide, an interfering RNA and an antisense RNA.

10. The method of claim 8, wherein the recombinant nucleic acid is provided in an exogenous stem cell that expresses the nucleic acid.

11. The method of claim 10, wherein detecting the contribution of a stem cell to the target tissue comprises detecting the contribution of the exogenous stem cell comprising the recombinant nucleic acid.

12. The method of claim 1, wherein the test treatment comprises causing damage to the target tissue.

13. The method of claim 12, wherein the test treatment comprises administering one or more of the following to the target tissue: radiation, exercise, a toxin, mechanical damage, cryodamage, damage mediated by immune cells or immune proteins.

14. The method of claim 13 wherein the toxin is selected from among: a membrane disrupting toxin, an excitotoxin and a degenerative toxin.

15. The method of claim 14, wherein the toxin is selected from among: notexin and cardiotoxin.

16. The method of claim 1, wherein the test treatment comprises an alteration in one or more of the following: subject diet, temperature and frequency of light exposure.

17. The method of claim 1, wherein the test treatment comprises administering an agent that mimics an aspect of a target tissue damage response.

18. The method of claim 17, wherein the agent is a pro-inflammatory agent.

19. The method of claim 1, wherein the test treatment comprises administering exogenous stem cells derived from a donor or subject having one or more of the following criterion: a selected genotype, a selected laboratory animal strain, a selected age and a selected disease state.

20. The method of claim 1, wherein the stem cell is selected from the group consisting of: a bone marrow derived stem cell, a hematopoietic stem cell and a myelomonocytic progenitor cell.

21. The method of claim 1, wherein the stem cell is selected from the group consisting of: a stem cell administered to the subject and a stem cell that is endogenous to the subject.

22. The method of claim 1, wherein the subject comprises a bone marrow derived stem cell having a tracking marker, and wherein detecting the contribution of the bone marrow derived stem cell to the target tissue comprises detecting the tracking marker in the target tissue.

23. The method of claim 22, wherein detecting the tracking marker in cells of the target tissue comprises cell sorting.

24. The method of claim 23, wherein cells are sorted, at least in part, on the basis of markers that differentiate mature cells and tissue-localized stem cells of the target tissue.

25. The method of claim 1, wherein the target tissue is selected from among: neural tissue, skeletal muscle tissue, heart muscle tissue, pancreatic tissue, cartilaginous tissue, adipose tissue and epithelial tissue.

26. The method of claim 1, wherein the subject is a transgenic animal comprising bone marrow stem cells having a tracking marker.

27. The method of claim 1, wherein the stem cells are obtained from a transgenic animal comprising stem cells having a tracking marker.

28. The method of claim 27, wherein the tracking marker is a reporter gene.

29. The method of claim 28, wherein expression of the reporter gene is regulated by a tissue- or cell-specific promoter.

30. A method for assessing the ability of a test criterion to alter the contribution of a stem cell to a target tissue in a subject, the method comprising:
a) detecting the contribution of a stem cell to the target tissue in a subject that has the test criterion; and
b) comparing the detected contribution to a reference;
wherein the stem cell is of a distinct developmental lineage from the target tissue.
